(19) United States
(12) Patent Application Publication
(10) Pub. No.: US 2017/0368104 A1
(43) Pub. Date: Dec. 28, 2017

(54) INDUCTION OF TOLERANCE IN LUNG ALLOGRAFT TRANSPLANTATION

(71) Applicant: Washington University, St. Louis, MO (US)

(72) Inventors: Alexander Sasha Krupnick, St. Louis, MO (US); Daniel Kreisel, St. Louis, MO (US); Andrew Gelman, St. Louis, MO (US)

(21) Appl. No.: 15/694,544

(22) Filed: Sep. 1, 2017

Related U.S. Application Data
(63) Continuation of application No. 14/334,660, filed on Jul. 17, 2014.
(60) Provisional application No. 61/847,552, filed on Jul. 17, 2013.
(60) Provisional application No. 61/907,721, filed on Nov. 22, 2013.

(51) Int. Cl. A61K 35/42; C12N 5/071; A61K 33/00; A61K 35/17; A61K 38/21; A61K 35/12

(52) U.S. Cl. CPC A61K 35/42; A61K 35/17; A61K 38/217; C12N 5/0688; A61K 33/00; C12N 2501/03; C12N 2501/24; A61K 2035/122

(57) ABSTRACT

The present disclosure relates to methods of inducing tolerance to lung allograft transplantation. These methods comprise increasing suppressor CD8⁺ T cells and/or suppressing deleterious CD8+ and CD4⁺ T cells.

### CROSS REFERENCE TO RELATED APPLICATIONS

This application is a continuation of U.S. Ser. No. 14/334,660, filed Jul. 17, 2014, which claims the priority of U.S. provisional application No. 61/847,552, filed Jul. 17, 2013, and U.S. provisional application No. 61/907,721, filed Nov. 22, 2013, each of which is hereby incorporated by reference in their entirety.

### GOVERNMENTAL RIGHTS

This invention was made with government support under K08CA131097, R01HL113931, K08HL083983, R01 HL094601, R01HL113436, HHSN268201000046C, and HL113931 awarded by the National Institutes of Health (NIH). The government has certain rights in the invention.

### FIELD OF THE INVENTION

The present disclosure relates to methods of inducing tolerance to lung allograft transplantation.

### BACKGROUND OF THE INVENTION

While transplantation has become an accepted form of therapy for end stage organ failure, formidable immunologic barriers limit long-term allograft survival. The currently accepted clinical immunosuppression protocols, consisting of life-long administration of calcineurin inhibitors, steroids and anti-metabolites, decrease immunosurveillance for both malignancies (Serraino et al, Eur J Cancer, 2007) and infectious diseases (Cervera et al., Enferm Infecc Microbiol Clin, 2012). Perioperative inhibition of lymphocyte activation through blockade of costimulatory pathways mediates acceptance of several types of allografts in murine models (Larsen et al, Nature, 1996; Banueolos et al, Transplantation, 2004; Markees et al, J Clin Invest, 1998). Clinical data point to the efficacy of costimulatory blockade for the treatment of autoimmune diseases in humans (Mease et al, Arthritis Rheum, 2011; Schiff, Rheumatology, 2011). Based on these data costimulatory blockade is being actively evaluated in human solid organ transplantation (Vincenti et al, Am J Transplant, 2011). This would be very advantageous for lung transplantation, where patients incur higher rates of graft loss compared to recipients of other solid organs (Kreisel et al, J Thorac Cardiovasc Surg, 2011) and suffer more infectious complications due to constant exposure of lung allografts to the external environment (Shah et al, Semin Respir Crit Care Med, 2010; Husain et al, Transplantation, 2009).

Alloreactive memory T cells are generated through previous blood transfusions, pregnancy or cross reactivity to viral or environmental antigens in a process known as heterologous immunity (Adams et al, J Clin Invest, 2003). When compared to naïve T cells, memory T cells have lower activation requirements and can rapidly trigger alloimmune responses through the synthesis of multiple inflammatory cytokines and cytolytic effector molecules (Adams et al, J Clin Invest, 2003). Furthermore, this cell population is relatively resistant to immunosuppression such as costimulatory blockade (Zhai et al, J Immunol, 2002; Trambley et al, J Clin Invest, 1999). Multiple studies have established that early infiltration of CD8⁺ memory T cells into allografts such as hearts, kidneys and livers, facilitates accelerated rejection and presents a barrier to immunosuppression-mediated long-term graft survival (Adams et al, J Clin Invest, 2003). Therefore, pre-clinical studies have focused on targeting this cell population in an effort to improve the survival of solid organ allografts such as kidneys (Koyama et al, Am J Transplant, 2007; Lo et al, Am J Transplant, 2011; Weaver et al, Nat Med, 2009).

According to the present disclosure, in contrast to what has been described for other organ transplants, early infiltration of CD8⁺ CD44^{hi} CD62L^{hi} CCR7⁺ central memory T cells is critical for lung allograft acceptance due to IFN-γ-mediated induction of local nitric oxide (NO). These findings identify a novel mechanism of allograft acceptance that challenges the currently accepted paradigm of global T cell depletion as induction therapy for lung transplant recipients.

### SUMMARY OF THE INVENTION

In an aspect, the present disclosure encompasses a method of inducing tolerance to a lung allograft in a subject. The method comprises administering CD8⁺ T cells to the lung allograft in an amount sufficient to suppress an alloimmune response in the subject.

In another aspect, the present disclosure encompasses a method of inducing tolerance to a lung allograft in a subject by increasing the level of nitric oxide in the lung allograph of a subject, where the level of nitric oxide is increased by administering CD8+ T cells to the lung allograft.

In another aspect the present disclosure encompasses a method of administering central memory T cells to an ex vivo graft, the method comprising, obtaining immune cells from a subject, culturing the immune cells in vitro, generating central memory T cells by stimulating the immune cells in vitro, and administering the central memory cells to the graft.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A-J depict cellular mechanisms of lung allograft rejection in the absence of immunosuppression. (FIG. 1A-C) Balb/c→Nude lung grafts remain ventilated and free of inflammation as demonstrated by gross appearance (FIG. 1A), histology (200× magnification, FIG. 1B) and ISHLT A grade (FIG. 1C). (FIG. 1D-F) Balb/c→B6 CD8^{-/-} lung grafts are acutely rejected within a week of transplantation, as evidenced by graft collapse due to loss of ventilation (FIG. 1D), by severe perivascular infiltration with inflammatory cells (FIG. 1E) and ISHLT A grade (FIG. 1F). (FIG. 1G-H) Perivascular infiltration in Balb/c→B6 CD8^{-/-} lung grafts was composed of CD4⁺ (FIG. 1G), but no CD8⁺ cells (FIG. 1H) (200× magnification). (FIG. 1I-K) Reconstitution of nude mice with B6 CD4⁺ T cells results in rejection of transplanted Balb/c grafts as evidenced by graft collapse due to loss of ventilation (FIG. 1I), by severe perivascular infiltration with inflammatory cells (FIG. 1J) and ISHLT A grade (FIG. 1K) (p=0.0039 compared to FIG. 1A-C by Mantel-Haenszel Chi-Square test). All gross and histological appearances as well as rejection grades represent grafts at 7 days after transplantation. TXP denotes transplanted graft and arrows point to perivascular infiltrates.

FIG. 2A-AA depict mechanisms of CSB-mediated lung acceptance. Balb/c lungs transplanted into (FIG. 2A-C) B6 or (FIG. 2D-F) CD4-depleted B6 recipients remain ventilated with minimal inflammation (gross (FIG. 2A, FIG. 2D), histology (FIG. 2B, FIG. 2E), ISHLT A rejection grade (FIG. 2C, FIG. 2F)). Rejection grades weren't significantly different (Mantel-Haenszel Chi-Square test (p=0.500)). Balb/c lungs transplanted into (FIG. 2G-FIG. 2I) CD8-depleted or (FIG. 2J-L) CD8^{-/-} B6 recipients are rejected, as evidenced by graft collapse due to loss of ventilation (FIG. 2G, FIG. 2J), by severe perivascular infiltration with inflammatory cells (FIG. 2H, FIG. 2K) and ISHLT A grade (FIG. 2I, FIG. 2L) (p<0.002 compared to FIG. 2A-C by Mantel-Haenszel Chi-Square test). (FIG. 2M-0) Acceptance is restored in B6 CD8^{-/-} mice after CD8⁺ T cell injection as demonstrated by gross appearance (FIG. 2M), histology (200× magnification, FIG. 2N) and ISHLT A grade (FIG. 2O). (p=0.613 vs. FIG. 2A-C by Mantel-Haenszel Chi-Square test). (FIG. 2P) While the proportion of CD4⁺Foxp3⁺ T cells isn't different in resting B6 vs. B6 CD8^{-/-} lungs, a higher abundance of graft-infiltrating CD4⁺Foxp3⁺ T cells is detectable in B6 compared to B6 CD8^{-/-} recipients (comparison between resting and transplanted lungs by ANOVA and comparison between B6 and B6 CD8^{-/-} groups by unpaired t-test). (FIG. 2Q-X) Adoptively transferred CD4⁺ CD45.1⁺ T cells. (FIG. 2O, FIG. 2R) Proliferation of B6 CD4+CD45.1⁺ T cells was greater after injection into B6 CD8^{-/-} (51.3±5%) than B6 wild-type (20.6±4%) recipients (FIG. 2W; p=0.0017 by unpaired t-test). Proliferating CD4⁺CD45.1⁺ T cells in B6 CD8^{-/-} recipients upregulated (FIG. 2S) CD27, (FIG. 2T) ICOS and (FIG. 2X) OX40, but not (FIG. 2U) CD28 or (V) CD154 compared to wild-type mice. (shaded grey=isotype controls; black lines=B6 wild-type; red lines=B6 CD8^{-/-} recipients) (FIG. 2Y, FIG. 2Z, FIG. 2AA) Inhibiting CD27-CD70, ICOS-ICOS-L and OX40-OX40-L in addition to blocking CD40-CD154 and CD28-B7 doesn't prevent rejection in the absence of CD8⁺ T cells as demonstrated by gross appearance (FIG. 2Y), histology (200× magnification, FIG. 2Z) and ISHLT A grade (FIG. 2AA) (p=0.00074 vs. FIG. 2A-C by Mantel-Haenszel Chi-Square test). Gross and histological appearances and rejection grades represent grafts on post-transplant day 7. TXP denotes graft and arrows point to perivascular infiltrates.

FIG. 3A-Z depict graft-infiltrating central memory CD8⁺CD44^{hi}CD62L^{hi}CCR7⁺ T cells play a critical role in downregulating alloimmune responses. (FIG. 3A) In vitro MLRs were established by isolating CD8⁺ T cells from CSB-treated Balb/c→B6 lung transplants and adding them as “regulators” to co-cultures of Balb/c splenocytes (stimulators) and CFSE-labeled B6 CD45.1⁺ T cells (responders). (FIG. 3B-G) After 5 days of co-culture the majority of B6 CD4⁺CD45.1⁺ T cells proliferate and blast as evidenced by size (forward scatter) (FIG. 3B, FIG. 3C). Proliferation and blasting is inhibited if CD8⁺ T cells isolated from accepting lung allografts are added to the MLRs (FIG. 3D, FIG. 3E). No inhibition is evident if CD8⁺ T cells are isolated from the spleens of accepting mice (FIG. 3F, FIG. 3G). Summary of proliferation and size (forward scatter) in the three groups is summarized in FIG. 3H, FIG. 3I with pair-wise comparison between groups performed by t-test. (FIG. 3J-Q) Proliferation and blasting of CD8⁺CD45.1⁺ T lymphocytes is inhibited by Balb/c→B6 lung allograft-derived CD8⁺ T cells (pair-wise comparison between groups performed by t-test as indicated). (FIG. 3R-V) Flow cytometry of CD8⁺ T lymphocytes in lung allografts of acceptors demonstrated few Foxp3⁺ or IL-10-producing cells. A large proportion of lung-resident CD8⁺ T cells had the capacity to produce IFN-γ and expressed a central memory phenotype (CD44^{hi}CD62L^{hi}CCR7⁺). (FIG. 3W-Z) Fewer cells in spleens of lung graft recipients had the capacity to produce IFN-γ and only few cells had a central memory T cell phenotype. Phenotype of CD8⁺ T cells representative of at least four separate experiments.

FIG. 4A-F depict central memory CD8⁺ T cells are abundant in the lung and can suppress alloimmune responses both in vitro and in vivo. (FIG. 4A) Compared to other solid organs such as heart, kidney and pancreas, the lung contains a relative abundance of CD8⁺ T lymphocytes including central memory cells. Central memory cells are defined as CD44^{hi}62L^{hi}, effector memory cells are defined as CD44^{hi}62L^{low}, and naïve cells are defined as CD44^{low}62L^{low}. Data is representative of four separate animals. (FIG. 4B) Freshly isolated central memory CD8⁺ T cells from resting B6 mice suppress proliferation of B6 CD4⁺CD45.1⁺ T cells stimulated with Balb/c splenocytes using similar methodology as described in FIG. 3A. Pair-wise comparison between proliferation profiles of responder CD4+CD45.1⁺ T cells in wells containing no CD8⁺ T cells, effector memory CD8⁺ T cells and central memory CD8⁺ T cells was performed by unpaired t-test. (FIG. 4C-E) Adoptive transfer of in vitro generated B6 anti-Balb/c central memory cells into B6 CD8^{-/-} recipients prevents rejection of Balb/c lung allografts after co-stimulatory blockade as demonstrated by gross appearance (FIG. 4C), histology (200× magnification, FIG. 4D) and ISHLT A grade (FIG. 4E) (p=0.751 compared to FIG. 2M-O by Mantel-Haenszel Chi-Square test). (FIG. 4F-H) Balb/c lungs are rejected by B6 CD8^{-/-} recipient mice reconstituted with in vitro generated anti-Balb/c CD8⁺ effector memory T lymphocytes despite costimulatory blockade as demonstrated by gross appearance (FIG. 4H), histology (200× magnification, FIG. 4G) and ISHLT A grade (FIG. 4F) (p=0.00105 compared to FIG. 2M-O by Mantel-Haenszel Chi-Square test).

FIG. 5A-Q depict central memory CD8⁺ T cells suppress through IFN-γ-mediated NO production. (FIG. 5A-C) Blocking IFN-γ prevents acceptance (gross (FIG. 5A), histology (FIG. 5B) and rejection grade (FIG. 5C)) (p=0.000258 vs. FIG. 2A-C by Mantel-Haenszel Chi-Square test). (FIG. 5D) In vitro proliferation of CD4+CD45.1⁺ T cells (CFSE) stimulated by Balb/c splenocytes in the presence of accepting allograft-derived CD8⁺ T cells after addition of IFN-γ-blocking (red) or control antibody (black). (n=3 separate experiments) (FIG. 5E) IFN-γ levels in allografts are significantly higher 4 days after transplantation into wild-type vs. CD8^{-/-} B6 recipients (n=4 each; unpaired t-test). (FIG. 5F-H) Injection of IFN-γ^{-/-} CD8⁺ T cells doesn't restore lung acceptance as demonstrated by gross appearance (FIG. 5F), histology (200× magnification, FIG. 5G) and ISHLT A grade (FIG. 5H) (p=0.0066 vs. FIG. 2M-O using Mantel-Haenszel Chi-Square test). (I-J) After five days the majority of CD4⁺CD45.1⁺ T cell “responders” are not viable if CD8⁺ T cells from accepting allografts are added (middle). CD4⁺CD45.1⁺ T cell viability (7-AAD) (FIG. 5I) and representative plots of CFSE vs. 7-AAD (FIG. 5J) (unpaired t-test). Gated on CD4⁺ CD45.1⁺ T lymphocytes. (FIG. 5K) CD4⁺ T cell proliferation (CFSE) and viability (7-AAD) in an MLR containing IFN-γR^{-/-} CD4⁺ T cell responders or IFN-γR^{-/-} antigen presenting cells (n=3). (FIG. 5L) CD4⁺ T cell proliferation after stimulation with plate-bound anti-CD3 and soluble anti-CD28 in the absence or presence of accepting allograft-derived CD8⁺ T cells (p=0.55 by unpaired t-test). (FIG. 5M) CD4⁺ T cell proliferation with inhibitors of amino acid metabolism, arginine or iNOS^{-/-} antigen presenting cells (ANOVA). (FIG. 5N) NO levels in resting lungs, allografts and right native lungs (n=3) (unpaired t-test) (FIG. 5O-Q) Balb/c lungs transplanted into CSB-treated iNOS^{-/-} B6 recipients as demonstrated by gross appearance (FIG. 5O), histology (200× magnification, FIG. 5P) and ISHLT A grade (FIG. 5Q) (p=0.00059 vs. FIG. 2A-C by Mantel-Haenszel Chi-Square test).

FIG. 6A-P depict chemokine receptor expression regulates CD8⁺ T cell-mediated lung acceptance. (FIG. 6A-C) CD8⁺ memory T cell infiltration into lung. Graft infiltration by pertussis toxin (PTX)-treated or untreated anti-donor (Balb/c) central memory (FIG. 6A), anti-third party (CBA/Ca) central memory (FIG. 6B) or anti-donor (Balb/c) effector memory (FIG. 6C) B6 CD8⁺CD45.1⁺ T cells (unpaired t-test). (FIG. 6D-F) Injection of CCR7^{-/-} CD8⁺ T cells doesn't restore allograft acceptance in B6 CD8^{-/-} recipients as demonstrated by gross appearance (FIG. 6D), histology (200× magnification, FIG. 6E) and ISHLT A grade (FIG. 6F) (p=0.00054 vs. FIG. 2M-O by Mantel-Haenszel Chi-Square test). (FIG. 6G) Immunosuppressed Balb/c→B6 CD8^{-/-} recipients reconstituted with wild-type B6 CD8⁺ T cells (n=8) had higher graft IFN-γ levels than those reconstituted with B6 CCR7^{-/-}CD8⁺ T cells (n=5) (day 4) (unpaired t-test). (FIG. 6H, FIG. 6I) Majority of recipient-derived graft-infiltrating CD11c⁺ cells in immunosuppressed Balb/c (CD45.2⁺)→B6 (CD45.1⁺) transplants express donor MHC class I (H-2K^{d}) (n=3). (FIG. 6J-P) Intravital two-photon microscopy demonstrating wild-type B6 CD8⁺ T cells (cyan), CCR7^{-/-} B6 CD8⁺ T cells (red) and CD11c±cells (green) in immunosuppressed Balb/c→B6 CD11c-EYFP allografts on day 4 (FIG. 6J). Collagen appears blue. Magnified views (FIG. 6L-P) show representative T cell movement over a 1-hour interval. Cyan tracks follow the movement of wild-type CD8⁺ T cells, whereas red tracks follow CCR7^{-/-} CD8⁺ T cells. Scale bars: 50 μm (FIG. 6J); 40 μm (FIG. 6L-P). FIG. 6L-P images are individual frames from a continuous time-lapse recording. Relative time displayed in min:sec. FIG. 6L-P are zoomed views from boxed regions. (FIG. 6K) Wild-type T cells (blue) have higher mean retention times (mostly associated with CD11c⁺ cells) than CCR7^{-/-} T cells (red) (top right). (23 vs. 16 minutes (p<0.001, t-test)). (Two independent experiments with similar results).

FIG. 7A-C depict images and a graph showing that Balb/c lung allografts (FIG. 7A) demonstrate little inflammation (FIG. 7B) with low ISHLT A grade (FIG. 7C) one week after transplantation into CSB-treated B6 mu Ig^{-/-} mice deficient in B cells. ISHLT A grade was compared by Mantel-Haenszel Chi-Square test to costimulatory blockade-treated Balb/c→B6 transplants described in FIG. 2A-C (p=0.365).

FIG. 8 depict a graph showing no differences in proportion of CD4⁺ T cells expressing Foxp3 are evident in the spleens of transplant recipients (comparison performed by unpaired t-test).

FIG. 9A-C depict images and a graph showing that IFNγ^{-/-} B6 mice do not accept Balb/c lung allografts despite co-stimulatory blockade as demonstrated by gross appearance (FIG. 9A), histology (200× magnification, FIG. 9B) and ISHLT A grade (FIG. 9C). ISHLT A grade was compared by Mantel-Haenszel Chi-Square test to co-stimulatory blockade-treated Balb/c→B6 transplants described in FIG. 2A-C with p=0.0006.

FIG. 10A-C depict graphs showing proliferating CD8⁺CD62L^{hi}CD44^{hi} central memory T cells, as determined by diminution of CFSE of adoptively transferred CD45.1⁺ congenic T cells, are detectable in lung allografts of costimulatory blockade-treated graft recipients (FIG. 10A). Increased proliferation of this cell population, however, is detectable in the absence of costimulatory blockade. Little proliferation is evident in either the spleen (FIG. 10C) or draining mediastinal lymph nodes (FIG. 10B) in immunosuppressed or non-immunosuppressed lung graft recipients. Data representative of three separate experiments analyzed by flow cytometry five days after adoptive transfer.

FIG. 11 depict a graph showing that despite the differences in lung allograft infiltration similar numbers of in vitro generated B6 CD45.1⁺ anti-Balb/c and anti-CBA CD8⁺ central memory T cells localize to the spleen after adoptive transfer (p=0.92 by unpaired t-test).

FIG. 12A-B depict an image and a graph showing B6 CCR7-deficient recipients reject Balb/c lung allografts despite costimulatory blockade as demonstrated by histology (200× magnification, FIG. 12A) and ISHLT A grade (FIG. 12B). Arrow points to perivascular inflammation. ISHLT A grade was compared by Mantel-Haenszel Chi-Square test to costimulatory blockade-treated Balb/c→B6 transplants described in FIG. 2A-C with p=0.00054.

### DETAILED DESCRIPTION OF THE INVENTION

The present disclosure is directed to methods of inducing tolerance to a lung allograft in a subject receiving a lung allograft. In one aspect, a method comprises increasing the level of nitric oxide in the lung allograft in an amount sufficient to suppress the alloimmune response in the lung allograft. In another aspect, the method comprises suppressing the alloimmune response to the lung allograft in the subject by increasing the number of CD8⁺ T cells in the lung allograft in an amount sufficient to suppress the alloimmune response in the lung allograft. The methods of the invention are in contrast to what has previously been described. Instead, the inventors have discovered that CD8⁺ CD44^{hi}CD62L^{hi}CCR7⁺ central memory T cells are critical to lung allograft acceptance due to IFN-γ mediated induction of nitric oxide. This discovery challenges the current paradigm of T cell depletion as induction therapy for lung transplants. Various embodiments of the methods are described herein.

An “allograft” is a transplant of an organ, tissue, bodily fluid or cell from one individual to a genetically non-identical individual of the same species. “Allograft rejection” as used herein refers to a partial or complete immune response to a transplanted cell, tissue, organ, or the like on or in a recipient of said transplant due to an immune response to an allograft. Allografts can be rejected through either a cell-mediated or humoral immune reaction of the recipient against histocompatability antigens present on the donor cells.

Allograft rejection may be a hyperacute rejection, an acute rejection, and/or a chronic rejection. Hyperacute rejection occurs within hours to days following transplantation and is mediated by a complement response in recipients with pre-existing antibodies to the donor. In hyperacute rejection, antibodies are observed in the transplant vasculature very soon after transplantation, leading to clotting, ischemia, and eventual necrosis and death. Hyperacute rejection is relatively rare due to pre-transplant screening (for example, for ABO blood type antibodies). Acute rejection occurs days to months following transplantation. Acute rejection is characterized by infiltration of the transplanted tissue by immune cells of the recipient, which carry out their effector function and destroy the transplanted tissue. Acute rejection is identified based on the presence of T-cell infiltration of the transplanted tissue, structural injury to the transplanted tissue, and injury to the vasculature of the transplanted tissue. Finally, chronic rejection occurs months to years following transplantation and is associated with chronic inflammatory and immune response against the transplanted tissue. Fibrosis is a common factor in chronic rejection of all types of organ transplants, often referred to as chronic allograft vasculopathy. Chronic rejection can typically be described by a range of specific disorders that are characteristic of the particular organ. For example, in lung transplants, such disorders include fibroproliferative destruction of the airway (bronchiolitis obliterans); in heart transplants or transplants of cardiac tissue, such as valve replacements, such disorders include fibrotic atherosclerosis; in kidney transplants, such disorders include, obstructive nephropathy, nephrosclerorsis, tubulointerstitial nephropathy; and in liver transplants, such disorders include disappearing bile duct syndrome. Chronic rejection can also be characterized by ischemic insult, denervation of the transplanted tissue, hyperlipidemia and hypertension associated with immunosuppressive drugs. One of skill in the art can diagnose allograft rejection type and severity in a transplant recipient.

A subject receiving an allograft may also be referred to as a transplant recipient or recipient. In some embodiments, the transplant recipient is a subject who has received an organ or other tissue transplant, such as one or more of a liver transplant, a kidney transplant, a heart transplant, a lung transplant, a bone marrow transplant, a small bowel transplant, a pancreas transplant, a trachea transplant, a skin transplant, a cornea transplant, or a limb transplant. In a specific embodiment, the transplant recipient has received a lung transplant.

As used herein, “subject” or “recipient” is used interchangeably. Suitable subjects include, but are not limited to, a human, a livestock animal, a companion animal, a lab animal, and a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In specific embodiments, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In a preferred embodiment, the subject is human.

Methods of the invention induce tolerance to an allograft in a subject. In a specific embodiment, methods of the invention induce tolerance to a lung allograft in a subject receiving a lung allograft. Immune tolerance, also referred to as immunological tolerance, describes a state of unresponsiveness of the immune system to substances or tissue that have the capacity to elicit an immune response. Transplant tolerance is defined as a state of donor-specific unresponsiveness without a need for ongoing pharmacologic immunosuppression. Transplantation tolerance could eliminate many of the adverse events associated with immunosuppressive agents. As such, induction of tolerance may result in improved receipt of an allograft. In an embodiment, induction of tolerance may be identified by a decrease in clinical symptoms of allograft rejection. In another embodiment, induction of tolerance may ameliorate or prevent the metabolic, inflammatory and proliferative pathological conditions or diseases associated with allograft transplantation. In still another embodiment, induction of tolerance may ameliorate or decrease or prevent the adverse clinical conditions or diseases associated with the administration of immunosuppressive therapy used to prevent allograft rejection. In still yet another embodiment, induction of tolerance may promote allograft survival. In a different embodiment, induction of tolerance may prevent relapses in patients exhibiting these diseases or conditions. The present method includes both medical therapeutic and/or prophylactic treatment to induce tolerance, as necessary.

Inducing tolerance to the allograft results in acceptance of the allograft. In a specific embodiment, inducing tolerance to the lung allograft results in acceptance of the lung allograft. In an embodiment, inducing tolerance to the allograft results in acceptance of the allograft for one day or longer from the time of receipt of the allograft. For example, inducing tolerance to the allograft results in acceptance of the allograft for 1 day, 2 days, 3 days, 4 days, 5 days, 6 days, or 7 days from the time of receipt of the allograft. In another embodiment, inducing tolerance to the allograft results in acceptance of the allograft for one week or longer from the time of receipt of the allograft. For example, inducing tolerance to the allograft results in acceptance of the allograft for about 1 week, about 1.5 weeks, about 2 weeks, about 2.5 weeks, about 3 weeks, about 3.5 weeks, about 4 weeks, about 4.5 weeks or about 5 weeks from the time of receipt of the allograft. In still another embodiment, inducing tolerance to the allograft results in acceptance of the allograft for one month or longer from the time of receipt of the allograft. For example, inducing tolerance to the allograft results in acceptance of the allograft for about 1 month, about 1.5 months, about 2 months, about 2.5 months, about 3 months, about 3.5 months, about 4 months, about 4.5 months, about 5 months, about 5.5 months, about 6 months, about 6.5 months, about 7 months, about 7.5 months, about 8 months, about 8.5 months, about 9 months, about 9.5 months, about 10 months, about 10.5 months, about 11 months, about 11.5 months or about 12 months from the time of receipt of the allograft. In yet still another embodiment, inducing tolerance to the allograft results in long-term acceptance of the allograft. For example, long-term acceptance may be about 1 year, about 2 years, about 3 years, about 4 years, about 5 years, about 10 years, about 15 years or about 20 years or more.

According to methods of the invention, the alloimmune response in the allograft is suppressed. In a specific embodiment, the alloimmune response in the lung allograft is suppressed. Specifically, the deleterious alloimmune response is suppressed. Alloimmunity is an immune response to foreign antigens (alloantigens) from members of the same species. In an alloimmune response, the allograft recipient's immune system rejects the allograft that has been introduced into/onto the recipient. In other words, the allograft recipient does not tolerate or maintain the organ, tissue or cell(s) that has been transplanted to it. An alloimmune response by the immune system of a tissue transplant generally occurs when the transplanted tissue is immunologically foreign. To facilitate acceptance of the allograft, the alloimmune response may be suppressed. Suppression of the alloimmune response may be referred to as immunosuppression. Immunosuppression is an act that reduces the activation or efficacy of the immune system.

In an aspect, an alloimmune response is suppressed by increasing the level of nitric oxide in the allograft in an amount sufficient to suppress the alloimmune response in the allograft. In a specific embodiment, an alloimmune response is suppressed by increasing the level of nitric oxide in the lung allograft in an amount sufficient to suppress the alloimmune response in the lung allograft. Nitric oxide, also referred to as nitrogen oxide, nitrogen monoxide, and/or NO is an important cellular signaling molecule. NO is one of the few gaseous signalling molecules known and is additionally exceptional due to the fact that it is a radical gas. It is a key vertebrate biological messenger, playing a role in a variety of biological processes. Nitric oxide, known as the ‘endothelium-derived relaxing factor’, or ‘EDRF’, is biosynthesized endogenously from L-arginine, oxygen, and NADPH by various nitric oxide synthase (NOS) enzymes. Reduction of inorganic nitrate may also serve to make nitric oxide. Independent of nitric oxide synthase, an alternative pathway, coined the nitrate-nitrite-nitric oxide pathway, elevates nitric oxide through the sequential reduction of dietary nitrate derived from plant-based foods. For the body to generate nitric oxide through the nitrate-nitrite-nitric oxide pathway, the reduction of nitrate to nitrite occurs in the mouth, by commensal bacteria, an obligatory and necessary step. Nitric oxide is also generated by phagocytes (monocytes, macrophages, and neutrophils) as part of the human immune response. Phagocytes are armed with inducible nitric oxide synthase (iNOS), which is activated by interferon-gamma (IFN-γ) as a single signal or by tumor necrosis factor (TNF) along with a second signal. On the other hand, transforming growth factor-beta (TGF-β) provides a strong inhibitory signal to iNOS, whereas interleukin-4 (IL-4) and IL-10 provide weak inhibitory signals.

An amount sufficient to suppress the alloimmune response will vary depending on the method of suppression, the disease or condition and its severity, the age of the subject, and so on. A sufficient amount can be determined by one of skill in the art. An amount sufficient to suppress the alloimmune response may be an amount sufficient to treat or inhibit a disease or conditions, such as allograft rejection in a transplant recipient. “Inhibiting” a condition or disease refers to inhibiting the full development of a condition or disease, for example allograft rejection in a subject. In contrast, “treatment” refers to a therapeutic intervention that ameliorates a sign or symptom of a condition or disease after it has begun to develop. A subject to be administered with an amount sufficient to inhibit or treat the disease or condition can be identified by standard diagnosing techniques for such a disorder, for example, basis of family history, or risk factor to develop the disease or disorder.

In an embodiment, the level of nitric oxide is increased. The level of nitric oxide may be increased at the time of receipt of the allograft. Alternatively, the level of nitric oxide may be increased prior to receipt of the allograft. For example, the level of nitric oxide may be increased about 5 days, about 4 days, about 3 days, about 2 days or about 1 day prior to receipt of the allograft. In another embodiment, the level of nitric oxide may be increased about 24 hours, about 22 hours, about 20 hours, about 18 hours, about 16 hours, about 14 hours, about 12 hours, about 10 hours, about 8 hours, about 7 hours, about 6 hours, about 5 hours, about 4 hours, about 3 hours, about 2 hours, or about 1 hour prior to receipt of the allograft. In still another embodiment, the level of nitric oxide may be increased about 45 min, about 30 min, about 20 min, about 15 min, about 10 min, about 5 min or about 1 min prior to receipt of the allograft. Further, the level of nitric oxide may be increased following receipt of the allograft. For example, the level of nitric oxide may be increased about 1 min, about 5 min, about 10 min, about 15 min, about 20 min, about 30 min, or about 45 min following receipt of the allograft. In another embodiment, the level of nitric oxide may be increased about 1 hour, about 2 hours, about 3 hours, about 4 hours, about 5 hours, about 6 hours, about 7 hours, about 8 hours, about 10 hours, about 12 hours, about 14 hours, about 16 hours, about 18 hours, about 20 hours, about 22 hours or about 24 hours following receipt of the allograft. The level of nitric oxide may be increased continuously or the level of nitric oxide may be increased hourly, daily, weekly or monthly.

In an aspect, the level of nitric oxide is significantly increased when compared to the level of nitric oxide in a normal tissue. In a specific embodiment, the level of nitric oxide is significantly increased when compared to the level of nitric oxide in a normal lung. For example, the level of nitric oxide may be increased to about double when compared to the level of nitric oxide in a normal lung. For example, the level of nitric oxide may be increased about 1.5 times, about 2 times, about 2.5 times, about 3 times, about 3.5 times, about 4 times, about 4.5 times, about 5 times, about 6 times, about 7 times, about 8 times, about 9 times or about 10 times or more when compared to the level of nitric oxide in a normal lung. In an embodiment, the level of nitric oxide in a normal lung may be the level of nitric oxide in the lung to be transplanted. In another embodiment, the level of nitric oxide in a normal lung may be the level of nitric oxide in the functional lung of the transplant recipient. In still another embodiment, the level of nitric oxide in a normal lung may be the average level of nitric oxide in a lung from an individual or group of individuals that have been shown to have normal lungs. A skilled artisan would be able to determine normal lungs. Methods of measuring the level of nitric oxide in a lung are known in the art. For example, the level of nitric oxide in a lung may be determined by measuring exhaled nitric oxide. Exhaled nitric oxide may be measured in a breath test. A normal level of nitric oxide may be about 20 to about 30 ppb. However, a skilled artisan would realize that several factors may influence the reference value. Non-limiting examples of factor that may influence the reference value include gender, smoking history, allergies, asthma, age, and/or height.

Any method of increasing the level nitric oxide in tissue may be used. In an embodiment, the level of nitric oxide may be increased by increasing production of nitric oxide by graft-infiltrating recipient cells. A graft-infiltrating recipient cell may be a cell that localizes to the allograft following transplant. For example, graft-infiltrating cells may be phagocytes such as monocytes, macrophages and/or neutrophils, CD8⁺ T cells, CD4⁺ T cells, and/or CD11c⁺ dendritic cells. In another embodiment, the level of nitric oxide may be increased by increasing production of nitric oxide by lung allograft cells. A lung allograft cell may be a cell that is transplanted into the recipient. Lung allograft cells may be phagocytes such as monocytes, macrophages and/or neutrophils, CD8⁺ T cells, CD4⁺ T cells, and/or CD11c⁺ dendritic cells. Methods of increasing production of nitric oxide by graft-infiltrating recipient cells or lung allograft cells may include, for example, increasing the number of graft-infiltrating recipient cells or lung allograft cells, stimulating graft-infiltrating recipient cells or lung allograft cells to produce nitric oxide through the addition of an exogenous compound, genetically modifying graft-infiltrating recipient cells or lung allograft cells to increase production of nitric oxide, or any other methods known to increase production of nitric oxide by cells.

In still another embodiment, the level of nitric oxide may be increased by administering a cytokine to the lung allograft. Any cytokine that increases the level of nitric oxide may be administered. For example, the cytokine may be IFN-γ or tumor necrosis factor (TNF). The production of IFN-γ leads to the induction of nitric oxide and downregulation of alloimmune responses as described in the Examples. In a specific embodiment, the level of nitric oxide may be increased by administering IFN-γ to the lung allograft. Methods of administering IFN-γ are known in the art. As IFN-γ is approved by the Food and Drug Administration (FDA), a skilled artisan would be able to determine route and dosing of administration.

In still yet another embodiment, the level of nitric oxide may be increased by administering inhaled nitric oxide to the lung allograft. Methods of administering inhaled nitric oxide are known in the art. For example, inhaled nitric oxide may be administered at concentrations of 5 to 80 ppm or, more preferably, at concentrations of 5 to 20 ppm. Alternatively, inhaled nitric oxide may be administered at a concentration as low as 10 ppb. Inhaled nitric oxide may be administered continuously. Alternatively, inhaled nitric oxide may be administered for a period of time such as hourly, daily, weekly or monthly.

In a different embodiment, the level of nitric oxide may be increased by administering a nitric oxide releasing compound to the lung allograft. Nitric oxide releasing compounds are known in the art. For example, a nitric oxide releasing compound or a nitric oxide donor may include, but is not limited to, a diazeniumdiolate (NONOate), a nitrate, a nitrite, BH₄, 1,5-Bis-(dihexyl-N-nitrosoamino)-2,4-dinitrobenzene, (±)-S-Nitroso-N-acetylpenicillamine (SNAP), S-Nitrosoglutathione (GSNO), streptozotocin (U-9889), NOC-12, NOC-18, NOC-9, 3-morpholinosydnonimine (SIN-1), NOR-1, DPTA NONOate, diethylamine NONOate, NOC-5, spermine NONOate, NOC-7, dephostatin, sodium nitroprusside dehydrate, JS-K, Piloty's acid, GEA 5583, PROLI NONOate, diethylamine NONOate/AM, NOR-5, SIN-1A/γCD complex, BEC, nicorandil, 4-phenyl-3-furoxancarbonitrile, GEA 5024, GEA 3162, PAPA NONOate, NOR-3, NOR-4, glycol-SNAP-1, β-gal NONOate, 4-(p-methoxyphenyl)-1,3,2-oxathiazolylium-5-olate, molsidomine, sulfo-NONOate disodium salt, 10-nitrooleate, DD1, 4-chloro-4-phenyl-1,3,2-oxathiozolylium-5-olate, 4-phenyl-1,3,2-oxathiazolylium-5-olate, 4-trifluoro-4-phenyl-1,3,2-oxathiazolylium-5-olate, BMN3, DD2, 3-(methylnitrosamino)propionitrile, S-nitrosocaptopril, NOR-2, V-PYRRO/NO, SE 175, NO-indomethacin, L-NMMA (citrate), and lansoprazole sulfone N-oxide. Nebulized NONOates may be a potential alternative to inhaled nitric oxide due to stability and prolonged half-life. A NONOate is a compound having the chemical formula R¹R²N—(NO⁻)—N═O, where R¹ and R² are alkyl groups. A nitrate is reduced to nitric oxide in the body. Non-limiting examples of nitrates include isosorbide dinitrate, isosorbide mononitrate, nitroglycerin (glyceryl trinitrate), pentaerythritol tetranitrate and dietary nitrate typically found in green, leafy vegetables. Nebulized nitrites may be nitric oxide donors, particularly in hypoxic conditions. BH₄ is a cofactor for nitric oxide synthase. Additionally, L-arginine and L-citrullline may be consumed by a recipient to increase nitric oxide. In a preferred embodiment, a nitric oxide releasing compound may be a NONOate, nitrite or nitrate.

In another embodiment, the level of nitric oxide may be increased by genetically modifying the allograft to increase nitric oxide production. Methods of genetically modifying cells are known and standard in the art. Cells may be genetically modified by gene addition or gene subtraction. For example, an allograft may be genetically modified to express various components of the nitric oxide pathway such as nitric oxide synthase. Alternatively, an allograft may be genetically modified to delete components that inhibit the nitric oxide pathway.

In another embodiment, the level of nitric oxide may be increased by inducing nitric oxide synthase activity in the allograft. In humans, nitric oxide is produced from L-arginine by three enzymes called nitric oxide synthases (NOS): inducible (iNOS), endothelial (eNOS), and neuronal (nNOS). The latter two are constantly active in endothelial cells and neurons respectively, whereas iNOS' action can be induced in states like inflammation (for example, by cytokines). In inflammation, several cells use iNOS to produce NO, including eosinophils. In a specific embodiment, the level of nitric oxide may be increased by inducing iNOS activity in the allograft. Any method to increase nitric oxide synthase activity may be used. For example, activation of the iNOS promoter may induce nitric oxide synthase activity in the allograft. Other methods of regulating the expression of nitric oxide synthase are known in the art. For example, see Pautz et al, 2010, 23(2):75-93.

In another embodiment, the level of nitric oxide may be increased by inhibiting nitric oxide synthase degradation. Any method of inhibiting nitric oxide synthase degradation may be used. Ubiquitin-proteasorne and calpain pathways are the major proteolytic systems identified that are responsible for degradation of nitric oxide synthase. As such, the level of nitric oxide may be increased by inhibiting proteolytic degradation pathways. Non-limiting examples of proteolytic degradation pathways include ubiquitin-proteasome pathway, calpain pathway, or autophagy-lysosomal pathway. Alternatively, administration of L-arginine may inhibit nitric oxide synthase degradation. L-arginine inhibits nitric oxide synthase degradation by substrate-induced stabilization of the enzyme that decreases proteolytic degradation of nitric oxide synthase. As such, any substrate that stabilizes nitric oxide synthase may be used to inhibit nitric oxide synthase degradation. In another embodiment, the level of nitric oxide may be increased by inhibiting nitric oxide-depleting enzymes in the allograft. The enzyme may be a nitric oxide dioxygenase or an arginase. Nitric oxide dioxygenase (EC 1.14.12.17) is an enzyme that catalyzes the conversion of nitric oxide (NO) to nitrate (NO₃⁻). Arginase may compete with NOS for their common substrate, L-arginine, and thus inhibit NO production.

In another embodiment, the level of nitric oxide may be increased by increasing the number of CD8⁺ T cells in the allograft. CD8⁺ T cells interact with antigen presenting cells resulting in the production of IFN-γ, which leads to the induction of nitric oxide and downregulation of alloimmune responses. Increasing the number of CD8⁺ T cells in the allograft is described in more detail below.

In an aspect, an alloimmune response is suppressed by suppression of CD4⁺ T cell proliferation. As such, suppression of CD4⁺ T cell proliferation may suppress the CD4⁺ T cell response. CD4 is expressed on mature Tₕ cells or T helper cells. T helper cells are a type of T cell that play an important role in the immune system, particularly in the adaptive immune system. They help the activity of other immune cells by releasing T cell cytokines. They are essential in B cell antibody class switching, in the activation and growth of cytotoxic T cells, and in maximizing bactericidal activity of phagocytes such as macrophages. Methods of suppressing CD4⁺ T cell proliferation are known in the art. For example, CD4⁺ T cells may be depleted using an antibody to CD4. In an exemplary embodiment, CD4⁺ T cells are depleted using the CD4 antibody GK1.5. Additionally, CD4⁺ T cells may be suppressed by cholera toxin B-subunit.

In another aspect, an alloimmune response is suppressed by suppression of deleterious CD8⁺ T cell proliferation. As such, suppression of deleterious CD8⁺ T cell proliferation may suppress the deleterious CD8⁺ T cell response. In an embodiment, a deleterious CD8⁺ T cell may be a CD8⁺ T cell that is not a central memory CD8⁺ T cell. In another embodiment, a deleterious CD8⁺ T cell may be a CD8⁺ T cell that is not a CD8⁺ CCR7⁺ T cell. In still another embodiment, a deleterious CD8⁺ T cell may be a CD8⁺ T cell that is not a CD8⁺ CD44^{hi} CD62L^{hi} CCR7⁺ T cell. As such, a deleterious CD8⁺ T cell may be an effector memory T cell. Effector memory CD8⁺ T cells are specialized antigen-experienced lymphocytes that traffic between blood and nonlymphoid tissues and are positioned to rapidly respond and execute effector functions at sites of infection. In an embodiment, a deleterious CD8⁺ T cell may be a CD8⁺ CCR7⁻ T cell. In another embodiment, a deleterious CD8+ T cell may be a CD8⁺ CD44^{hi}CD62L^{low} T cell. In still another embodiment, a deleterious CD8+ T cell may be a CD8⁺ CCR7⁺ CD62L⁻ T cell.

According to the invention, CD8⁺ T cells may suppress CD4⁺ T cell proliferation. Specifically, central memory CD8⁺ T cells may suppress CD4⁺ T cell proliferation. As such, increasing the number of CD8⁺ T cells in the allograft may suppress the alloimmune response in the allograft. Specifically, increasing the number of central memory CD8⁺ T cells in the allograft may suppress the alloimmune response in the allograft. Central memory CD8⁺ T cells may also be referred to as regulatory central memory T cells or suppressive CD8⁺ T cells. In a specific embodiment, increasing the number of central memory CD8⁺ T cells in the lung allograft may suppress the alloimmune response in the lung allograft. CD8 is expressed on cytotoxic T cells. CD8⁺ T cells are recognized as cytotoxic T cells once they become activated and are generally classified as having a pre-defined cytotoxic role within the immune system. CD8⁺ T cells of the invention may be CD8⁺ central memory T cells. More specifically, CD8⁺ T cells may be CD8⁺ CCR7⁺ central memory T cells. In another embodiment, CD8⁺ T cells may be CD8⁺ CCR7⁺ CD62L^{hi} central memory T cells. In a specific embodiment, the CD8⁺ T cells may be CD8⁺ CD44^{hi} CD62L^{hi} CCR7⁺ central memory T cells.

In an embodiment, the number of CD8⁺ T cells is increased. In a specific embodiment, the number of central memory CD8⁺ T cells is increased. The number of CD8⁺ T cells may be increased at the time of receipt of the allograft. Alternatively, the number of CD8⁺ T cells may be increased prior to receipt of the allograft. For example, the number of CD8⁺ T cells may be increased about 5 days, about 4 days, about 3 days, about 2 days or about 1 day prior to receipt of the allograft. In another embodiment, the number of CD8⁺ T cells may be increased about 24 hours, about 22 hours, about 20 hours, about 18 hours, about 16 hours, about 14 hours, about 12 hours, about 10 hours, about 8 hours, about 7 hours, about 6 hours, about 5 hours, about 4 hours, about 3 hours, about 2 hours, or about 1 hour prior to receipt of the allograft. In still another embodiment, the number of CD8⁺ T cells may be increased about 45 min, about 30 min, about 20 min, about 15 min, about 10 min, about 5 min or about 1 min prior to receipt of the allograft. Further, the number of CD8⁺ T cells may be increased following receipt of the allograft. For example, the number of CD8⁺ T cells may be increased about 1 min, about 5 min, about 10 min, about 15 min, about 20 min, about 30 min, or about 45 min following receipt of the allograft. In another embodiment, the number of CD8⁺ T cells may be increased about 1 hour, about 2 hours, about 3 hours, about 4 hours, about 5 hours, about 6 hours, about 7 hours, about 8 hours, about 10 hours, about 12 hours, about 14 hours, about 16 hours, about 18 hours, about 20 hours, about 22 hours or about 24 hours following receipt of the allograft.

In an aspect, the number of CD8⁺ T cells is significantly increased when compared to the number of CD8⁺ T cells in a normal tissue. In a specific embodiment, the number of central memory CD8⁺ T cells is significantly increased when compared to the number of central memory CD8⁺ T cells in a normal lung. For example, the number of CD8⁺ T cells may be increased to about double when compared to the number of CD8⁺ T cells in a normal lung. For example, the number of CD8⁺ T cells may be increased about 1.5 times, about 2 times, about 2.5 times, about 3 times, about 3.5 times, about 4 times, about 4.5 times, about 5 times, about 6 times, about 7 times, about 8 times, about 9 times or about 10 times or more when compared to the number of CD8⁺ T cells in a normal lung. In an embodiment, the number of CD8⁺ T cells in a normal lung may be the number of CD8⁺ T cells in the lung to be transplanted. In another embodiment, the number of CD8⁺ T cells in a normal lung may be the number of CD8⁺ T cells in the functional lung of the transplant recipient. In still another embodiment, the number of CD8⁺ T cells in a normal lung may be the average number of CD8⁺ T cells in a lung from an individual or group individuals that have been shown to have normal lungs. A skilled artisan would be able to determine normal lungs. Methods of measuring the number of CD8⁺ T cells in a lung are known in the art. For example, the number of CD8⁺ T cells in a lung may be determined by histology or flow cytometry of a biopsy sample.

In an embodiment, the number of CD8⁺ T cells may be increased by perfusing the lung allograft with the CD8⁺ T cells. In a specific embodiment, the number of central memory CD8⁺ T cells may be increase by perfusing the lung allograft with the CD8⁺ T cells. The CD8⁺ T cells may be recipient CD8⁺ T cells, donor CD8⁺ T cells, or a combination thereof. CD8⁺ T cells may be isolated for perfusion by methods known in the art. The cells for perfusion may be subjected to selection and purification, which may include both positive and negative selection methods, to obtain a substantially pure population of cells. In one aspect, fluorescence activated cell sorting (FACS), also referred to as flow cytometry, is used to sort and analyze the different cell populations. Cells having the cellular markers specific for CD8⁺ T cells are tagged with an antibody, or typically a mixture of antibodies, that bind the cellular markers. Each antibody directed to a different marker is conjugated to a detectable molecule, particularly a fluorescent dye that can be distinguished from other fluorescent dyes coupled to other antibodies. A stream of tagged or “stained” cells is passed through a light source that excites the fluorochrome and the emission spectrum from the cells detected to determine the presence of a particular labeled antibody. By concurrent detection of different fluorochromes, also referred to in the art as multicolor fluorescence cell sorting, cells displaying different sets of cell markers may be identified and isolated from other cells in the population. Other FACS parameters, including by way of example and not limitation, side scatter (SSC), forward scatter (FSC), and vital dye staining (e.g., with propidium iodide) allow selection of cells based on size and viability. General guidance on fluorescence activated cell sorting is described in, for example, Shapiro, H. M., Practical Flow Cytometry, 4th Ed., Wiley-Liss (2003) and Ormerod, M. G., Flow Cytometry: A Practical Approach, 3rd Ed., Oxford University Press (2000).

Another method of isolating CD8⁺ T cells for perfusion uses a solid or insoluble substrate to which is bound antibodies or ligands that interact with specific cell surface markers. In immunoadsorption techniques, cells are contacted with the substrate (e.g., column of beads, flasks, magnetic particles) containing the antibodies and any unbound cells removed. Immunoadsorption techniques can be scaled up to deal directly with the large numbers of cells in a clinical harvest. Suitable substrates include, by way of example and not limitation, plastic, cellulose, dextran, polyacrylamide, agarose, and others known in the art (e.g., Pharmacia Sepharose 6 MB macrobeads). When a solid substrate comprising magnetic or paramagnetic beads is used, cells bound to the beads can be readily isolated by a magnetic separator (see, e.g., Kato, K. and Radbruch, A., Cytometry 14(4):38492 (1993)). Affinity chromatographic cell separations typically involve passing a suspension of cells over a support bearing a selective ligand immobilized to its surface. The ligand interacts with its specific target molecule on the cell and is captured on the matrix. The bound cell is released by the addition of an elution agent to the running buffer of the column and the free cell is washed through the column and harvested as a homogeneous population. As apparent to the skilled artisan, adsorption techniques are not limited to those employing specific antibodies, and may use nonspecific adsorption. For example, adsorption to silica is a simple procedure for removing phagocytes from cell preparations.

FACS and most batch wise immunoadsorption techniques can be adapted to both positive and negative selection procedures (see, e.g., U.S. Pat. No. 5,877,299). In positive selection, the desired cells are labeled with antibodies and removed away from the remaining unlabeled/unwanted cells. In negative selection, the unwanted cells are labeled and removed. Another type of negative selection that can be employed is use of antibody/complement treatment or immunotoxins to remove unwanted cells.

It is to be understood that the isolation of cells also includes combinations of the methods described above. A typical combination may comprise an initial procedure that is effective in removing the bulk of unwanted cells and cellular material. A second step may include isolation of cells expressing a marker common to CD8⁺ T cell populations by immunoadsorption on antibodies bound to a substrate. For example, magnetic beads containing anti-CD8 antibodies are able to bind and capture CD8⁺ T cells that express the CD8 antigen. An additional step providing higher resolution of different cell types, such as FACS sorting with antibodies to a set of specific cellular markers, can be used to obtain substantially pure populations of the desired cells. Another combination may involve an initial separation using magnetic beads bound with anti-CD8 antibodies followed by an additional round of purification with FACS. Cells may be purified such that the isolated population is purified CD8⁺ central memory T cells. In a specific embodiment, purified CD8⁺ central memory T cells are isolated CD8⁺ CCR7⁺ central memory T cells. In another specific embodiment, purified CD8⁺ central memory T cells are isolated CD8⁺ CD44^{hi}CD62L^{hi}CCR7⁺ central memory T cells. Purification of cells may result in a substantially pure population of CD8⁺ central memory T cells. The term “substantially pure”, may be used herein to describe a purified population of CD8⁺ T cells that is enriched for CD8⁺ central memory T cells, but wherein the population of CD8⁺ central memory T cells are not necessarily in a pure form. Accordingly, a “substantially pure cell population” refers to a population of cells having a specified cell marker characteristic and differentiation potential that is at least about 50%, preferably at least about 75-80%, more preferably at least about 85-90%, and most preferably at least about 95% of the cells making up the total cell population. Thus, a “substantially pure cell population” refers to a population of cells that contain fewer than about 50%, preferably fewer than about 20-25%, more preferably fewer than about 10-15%, and most preferably fewer than about 5% of cells that do not display a specified marker characteristic and differentiation potential under designated assay conditions.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Both CD4⁺ and CD8⁺ T Lymphocytes can Mediate Lung Allograft Rejection

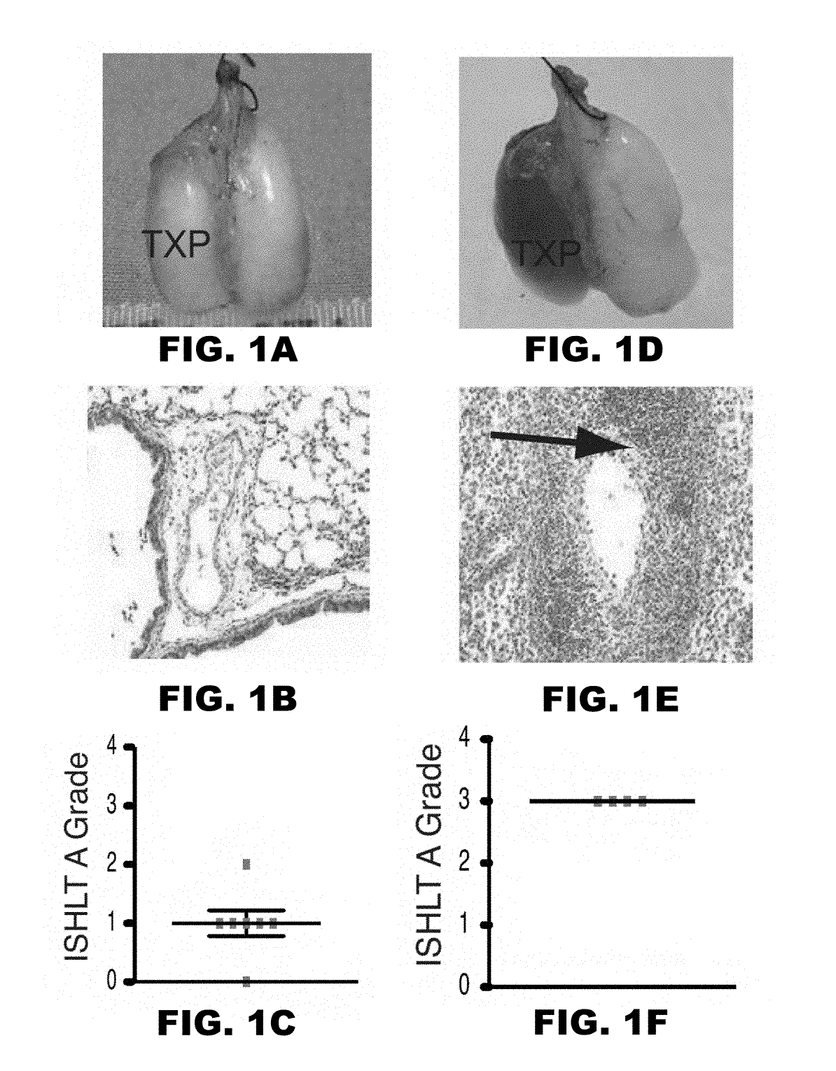

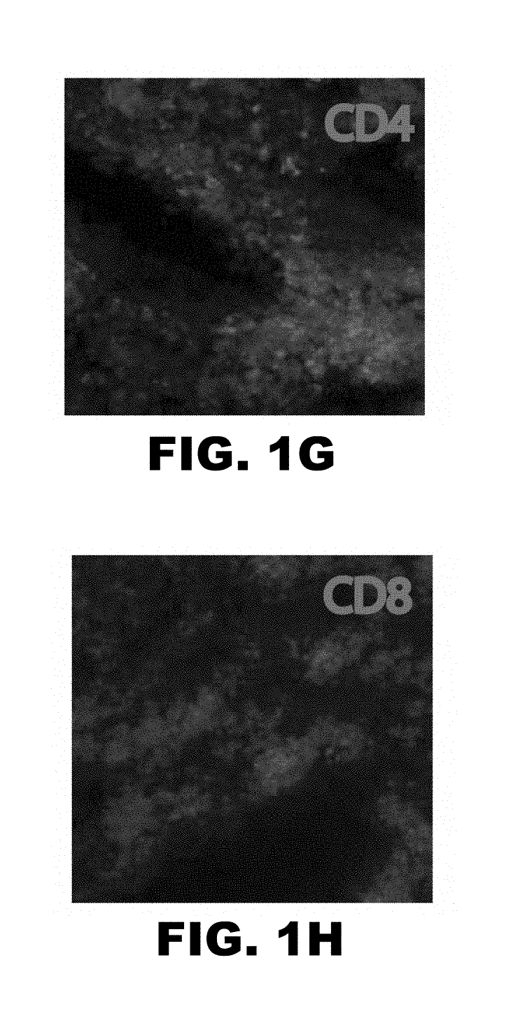

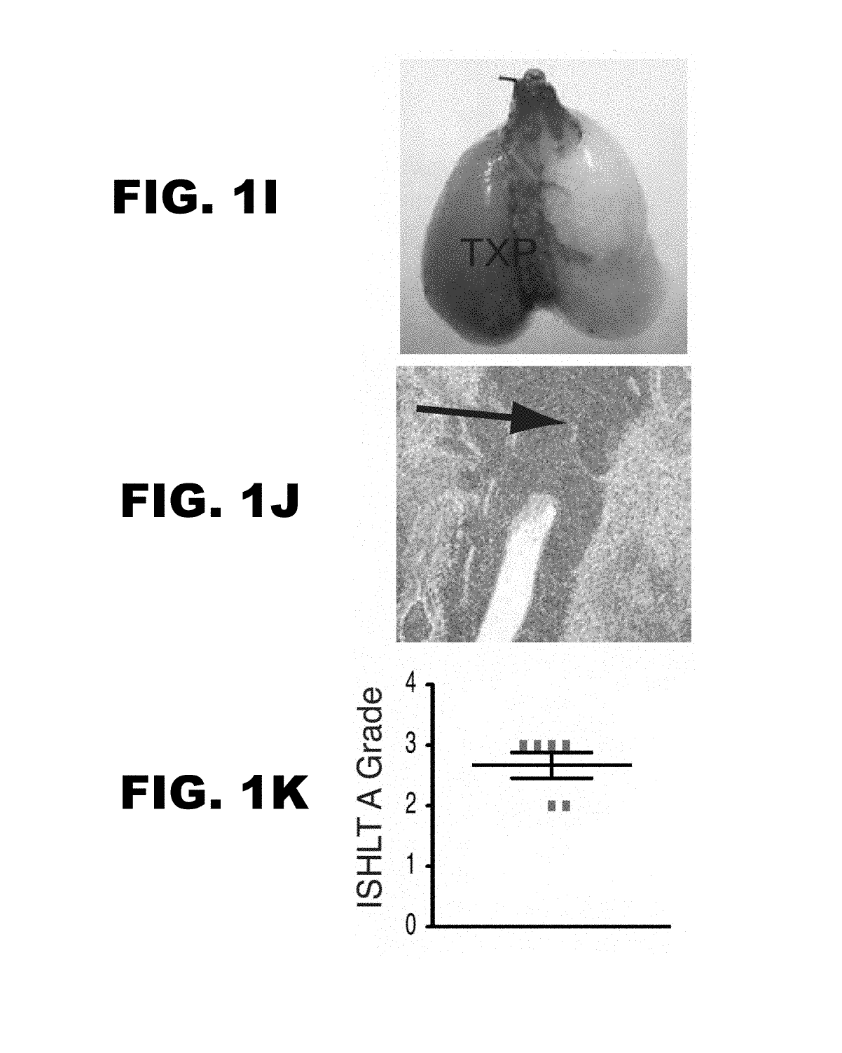

Lung allograft rejection is diagnosed and graded based on histological findings of cellular infiltrates (25). A wide variety of leukocytes, including B cells, macrophages, neutrophils and natural killer cells, have been shown to contribute to rejection of solid organs (26-28) and to date it has not been established whether T lymphocytes are necessary to mediate lung allograft rejection. To address this issue, Balb/c lungs were transplanted into allogeneic athymic nude mice and it was determined that, in contrast to wild-type recipients (29), these grafts remain ventilated with little inflammation one week post-transplantation (FIG. 1A-C) and long-term (30). It has been previously shown that, unlike the case for cardiac transplants, lung allografts can be rejected in the absence of CD4⁺ T cells (31). To test whether CD8⁺ T cells are essential for the rejection of pulmonary allografts, Balb/c lungs were transplanted into CD8⁺ T cell-deficient B6 recipients (B6 CD8^{-/-} from here on) and histological changes of severe acute rejection with perivascular lymphocytic infiltrates comparable to those seen in CD8⁺ T cell-sufficient B6 recipients were noted (FIG. 1D-F). Immunostaining of these grafts demonstrated extensive infiltration with CD4⁺ T cells and no detectable CD8⁺ T cells (FIG. 1G, FIG. 1H). Furthermore, reconstitution of nude mice with CD4⁺ T cells led to rejection of lung allografts (32) (FIG. 1I-K). Taken together, it was concluded that thymically derived T lymphocytes are necessary for lung allograft rejection and that either CD4⁺ or CD8⁺ T cells are sufficient to mediate this process.

### Example 2. CD8⁺ T Lymphocytes are Critical for Lung Allograft Acceptance

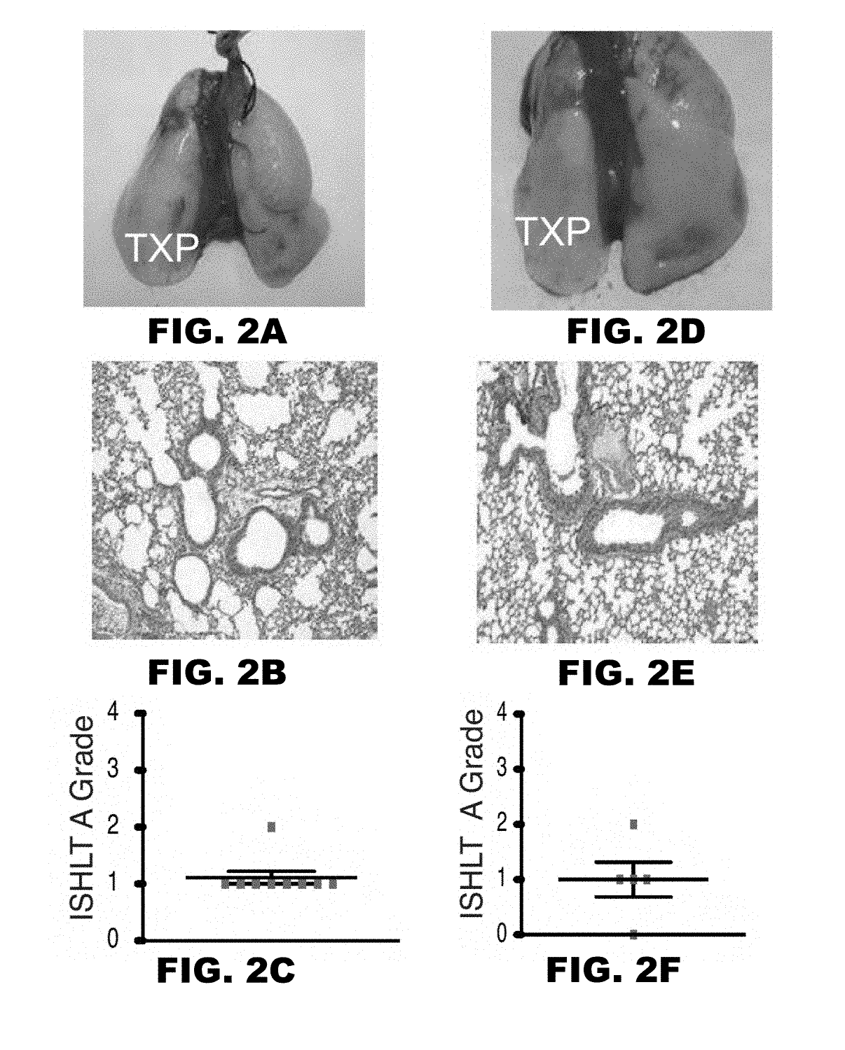

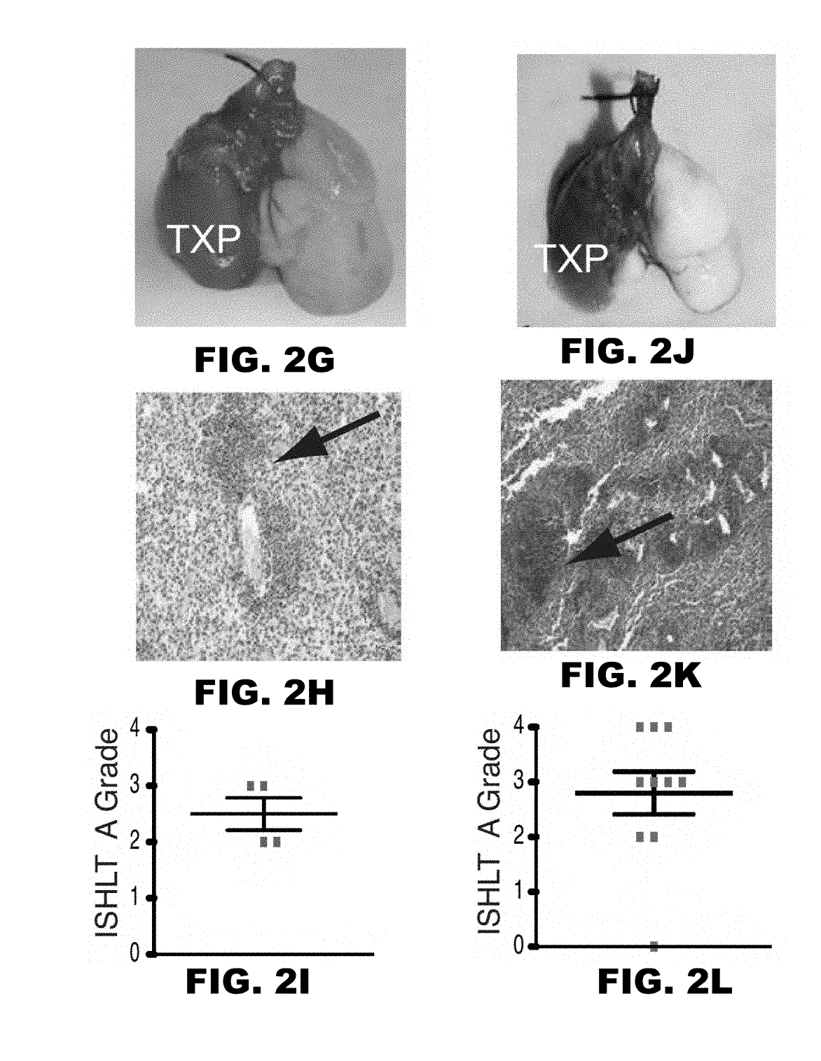

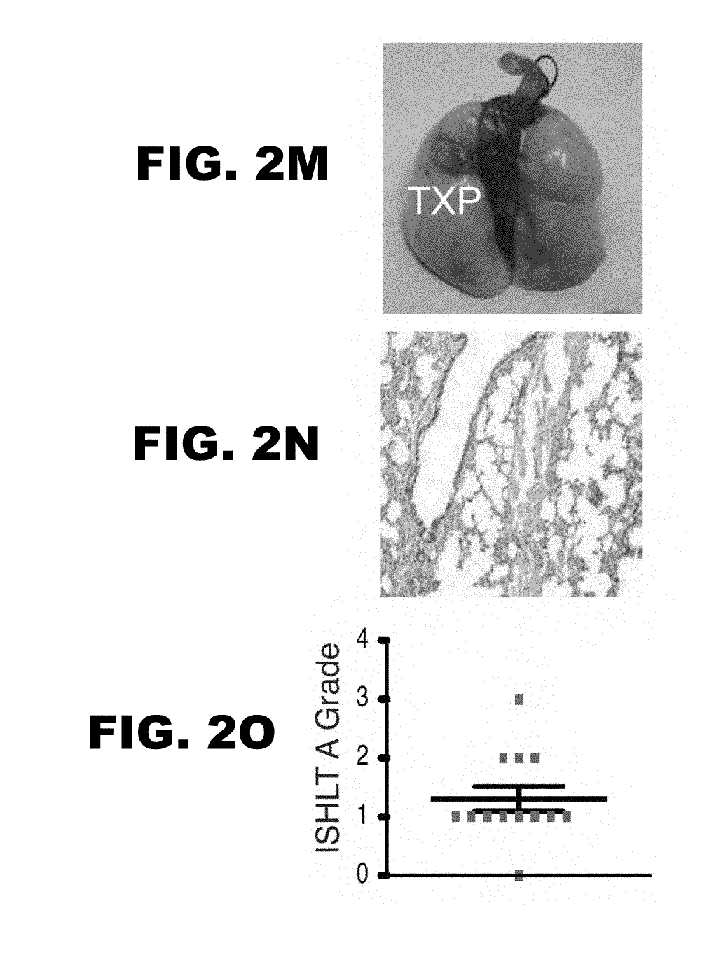

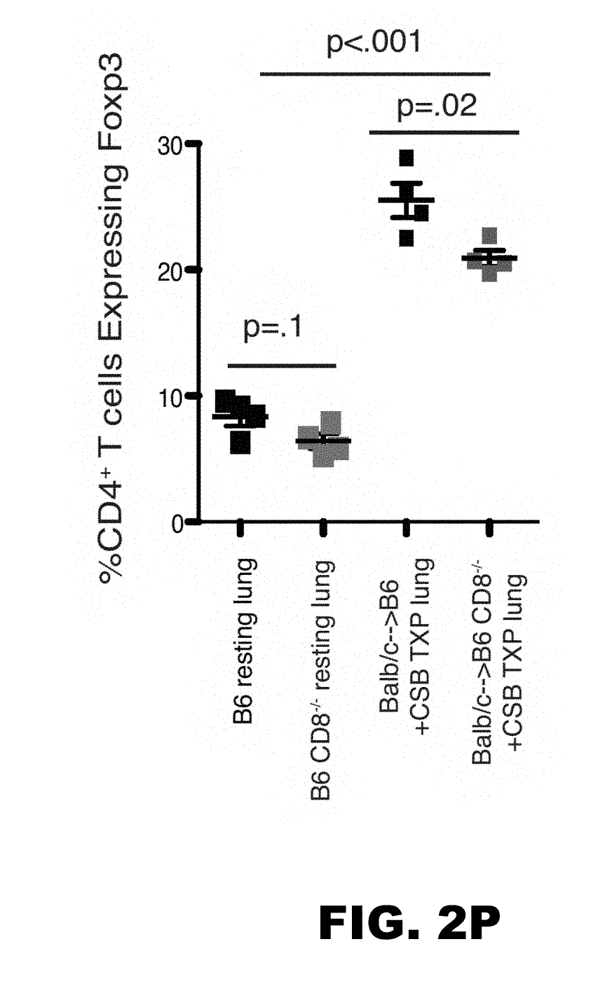

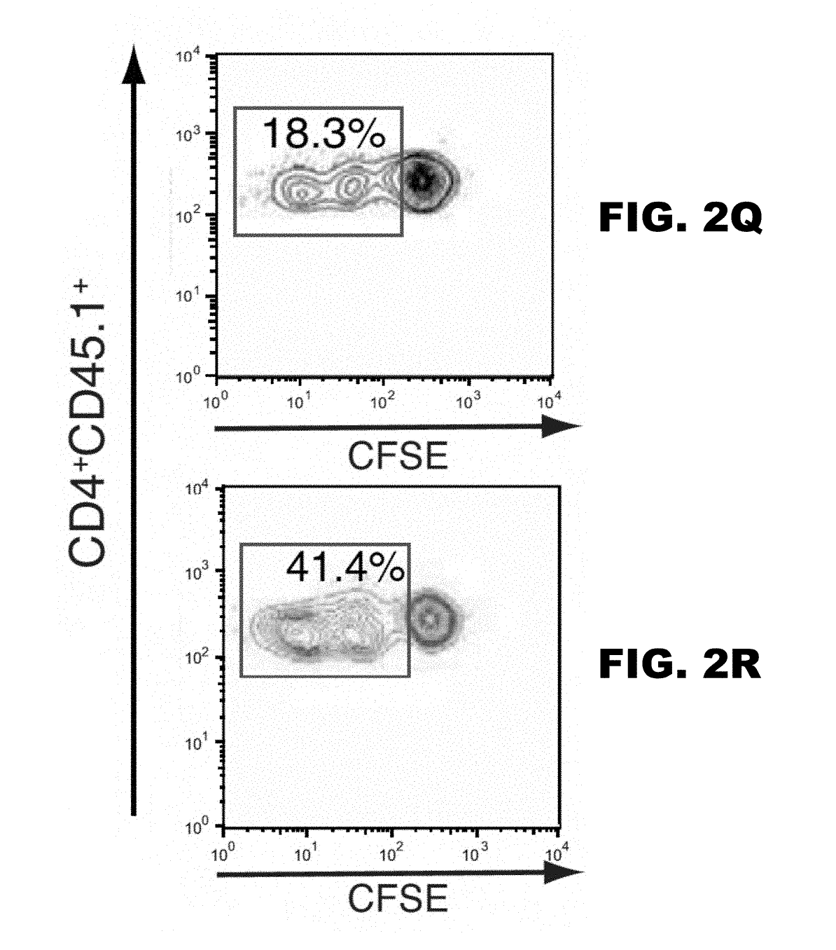

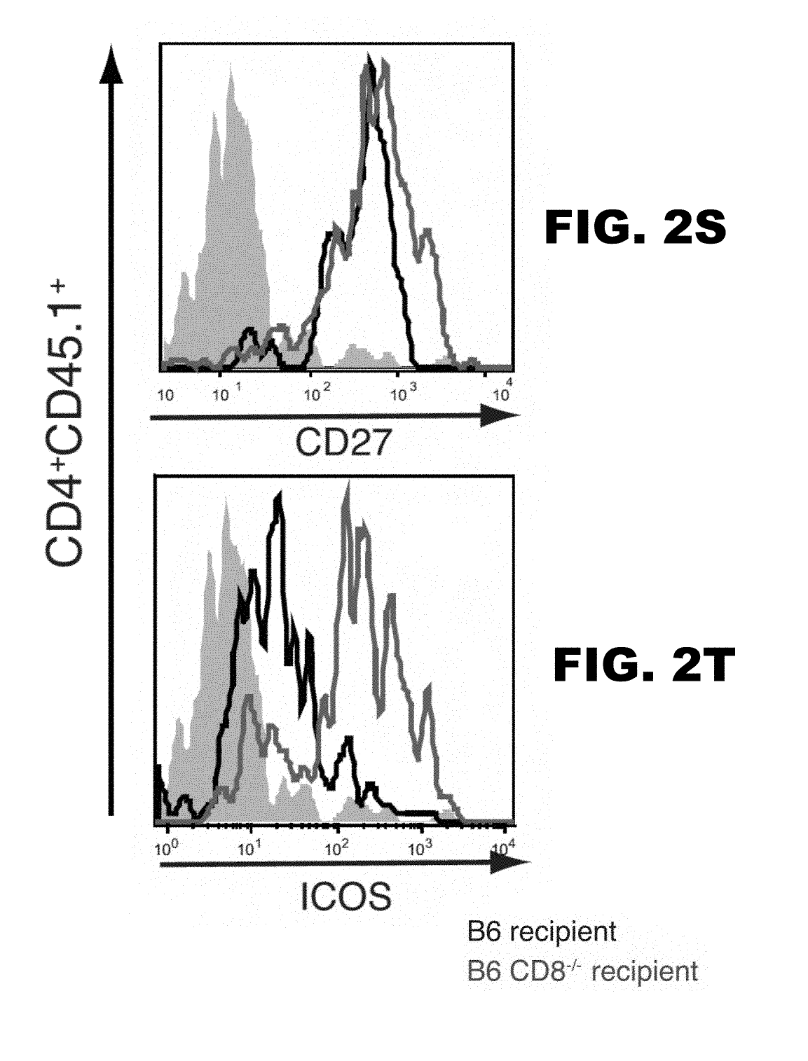

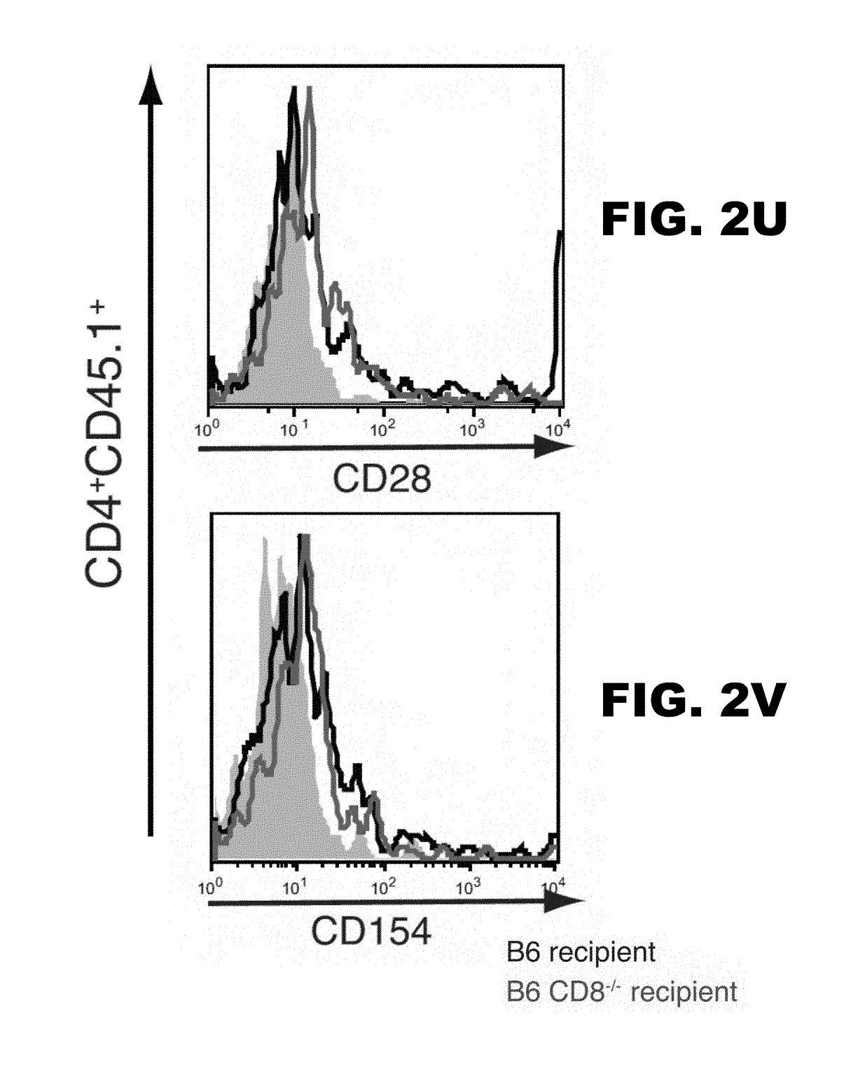

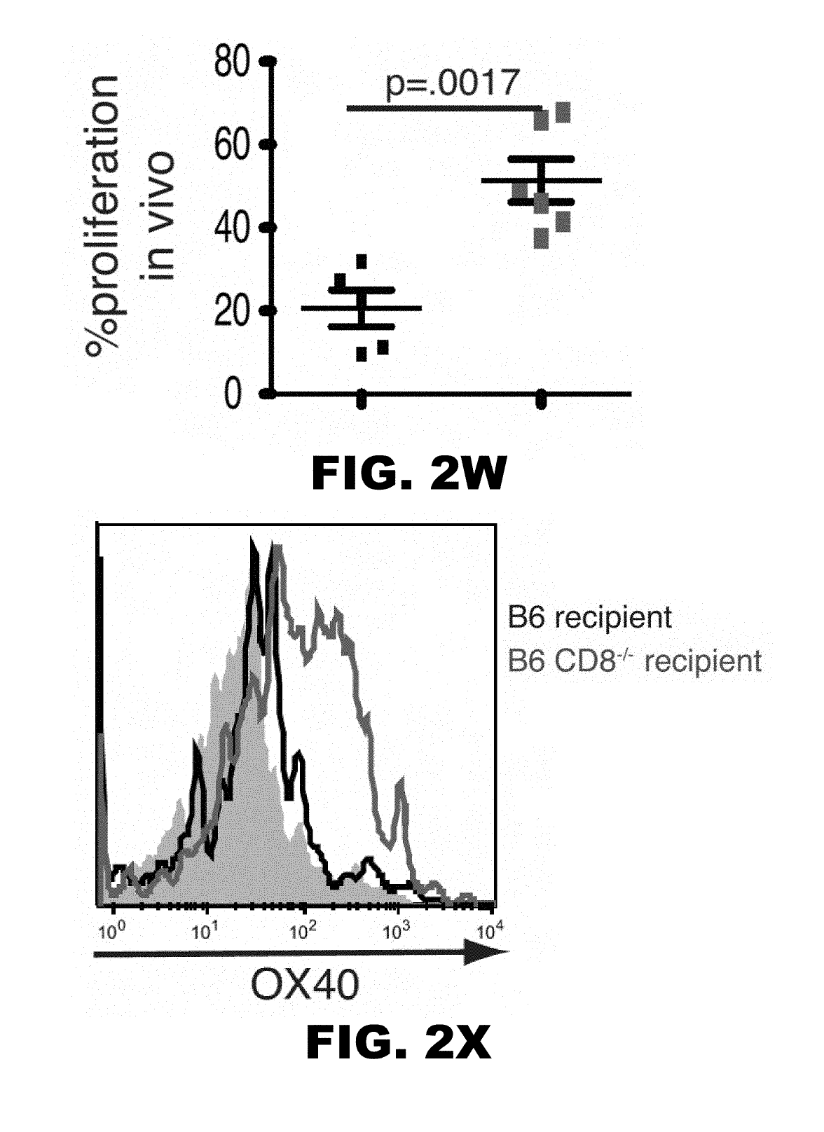

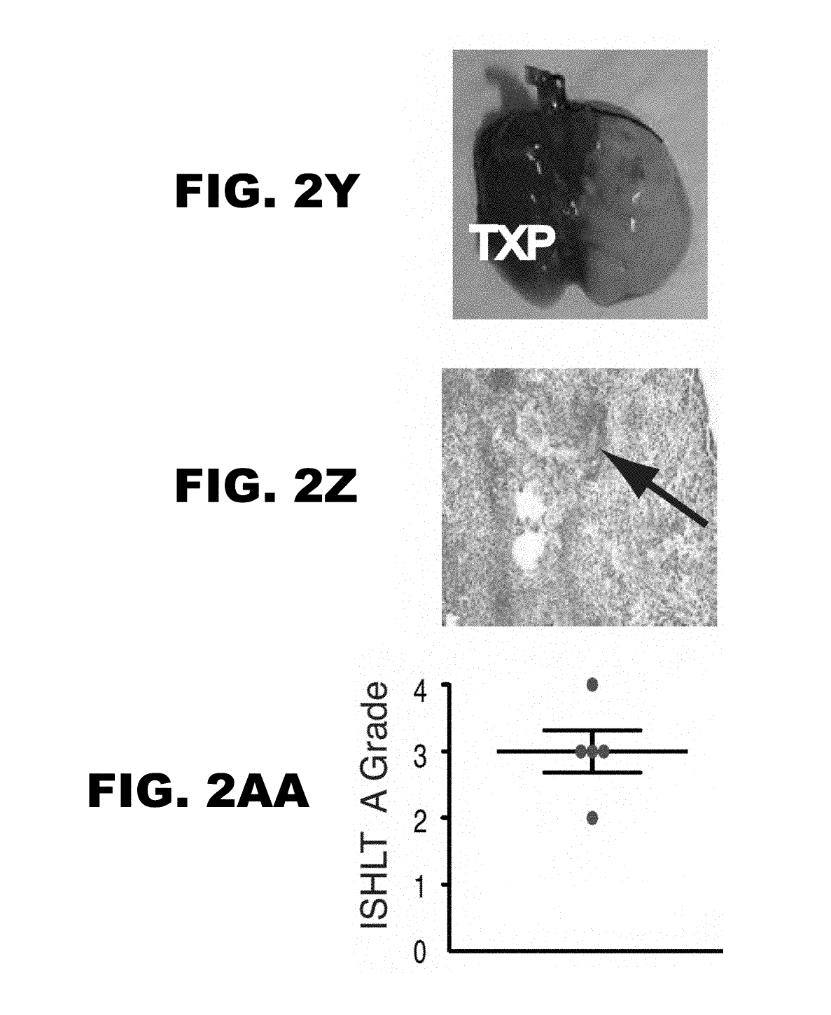

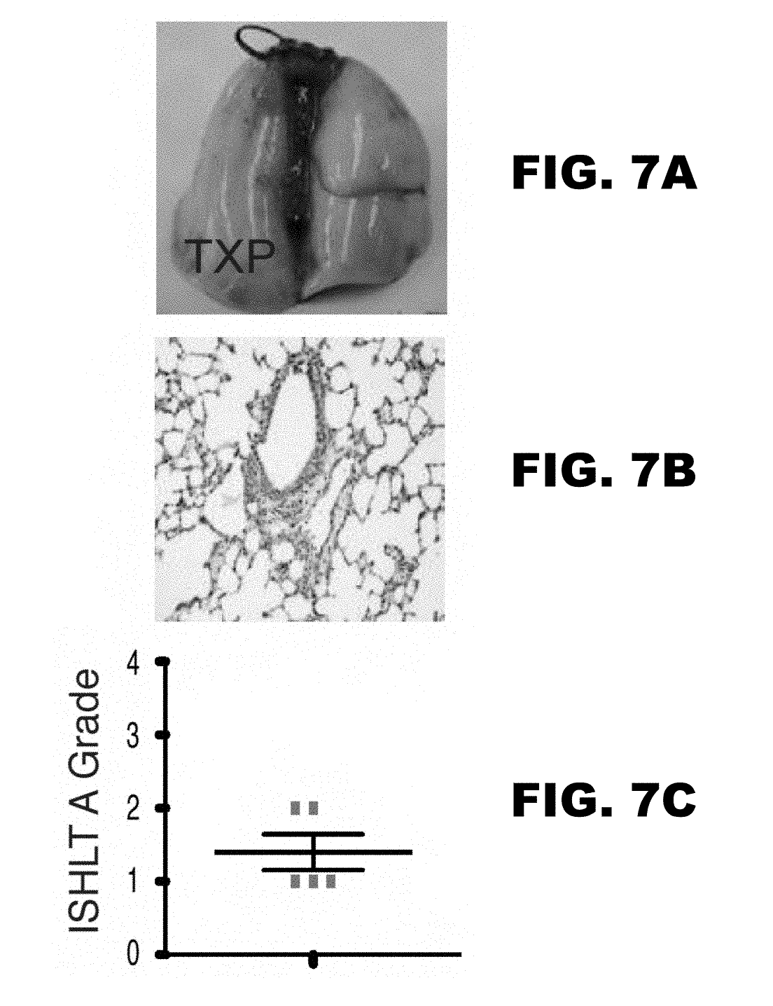

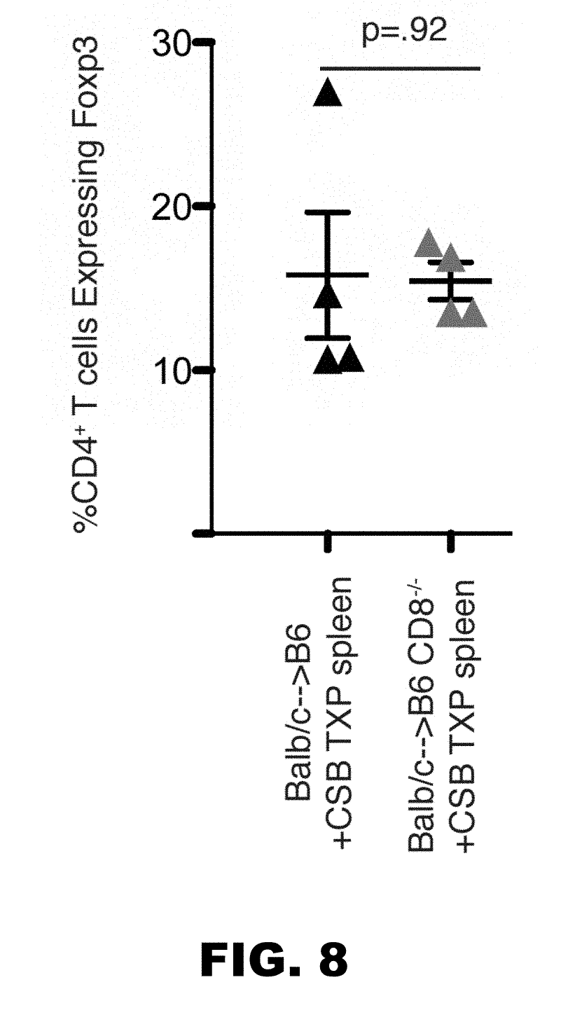

It has been demonstrated that immunosuppression through blockade of the CD28/B7 and CD40/CD154 costimulatory pathways leads to long-term lung allograft acceptance in the Balb/c→B6 (31, 33) as well as other strain combinations (30). Regulatory CD4⁺ T cells have been shown to play a critical role in costimulatory blockade-mediated acceptance of heart, skin and islet allografts as well as amelioration of autoimmune diseases (4, 5, 34-38). Recipient bulk CD4⁺ T cell antibody-mediated depletion, however, did not affect the fate of immunosuppressed lung allografts with rejection grades comparable to wild-type costimulatory blockade-treated hosts (FIG. 2A-F). While regulatory B cells have been described in some models of solid organ transplantation (39), Balb/c lung allograft acceptance in B6 B cell-deficient mice was still induced (FIG. 7A-C). Surprisingly, pulmonary allografts transplanted into costimulatory blockade-treated B6 CD8⁺ T cell-depleted (FIG. 2G-I) or B6 CD8^{-/-} mice (FIG. 2J-L) were acutely rejected with severe graft inflammation. The appearances of these grafts and rejection grades were similar to what has previously reported for lungs transplanted into non-immunosuppressed allogeneic recipients (40). Adoptive transfer of wild-type B6 CD8⁺ T cells into immunosuppressed B6 CD8^{-/-} recipients restored acceptance of Balb/c lungs (FIG. 2M-O). Seven days after engraftment, an increase in CD4⁺Foxp3⁺ T cells in lung allografts of immunosuppressed Balb/c→B6 and Balb/c→B6 CD8^{-/-} recipients compared to lungs from untransplanted controls was observed (FIG. 2P). However, the proportion of CD4⁺ T cells expressing Foxp3 in lung allografts was higher in the presence of CD8⁺ T cells. No such differences were evident in spleens of transplanted mice (FIG. 8). Thus, CD8⁺ T cells play a critical role in mediating lung allograft acceptance.

Based on the finding that CD4⁺ T cells can trigger lung rejection in the absence of CD8⁺ T cells, CD4⁺ T lymphocyte responses in the presence or absence of CD8⁺ T cells were evaluated. CFSE-labeled congenic B6 CD45.1⁺CD4⁺ T cells were injected into costimulatory blockade-treated B6 wild-type or B6 CD8^{-/-} recipient mice at the time of Balb/c lung transplantation and enhanced proliferation of this cell population in B6 CD8^{-/-} compared to B6 wild-type hosts was observed (FIG. 2Q, FIG. 2R, FIG. 2W). After transfer into B6 CD8^{-/-} recipients, several other costimulatory receptors such as CD27, ICOS and OX40 were upregulated on CD4⁺ T cells compared to B6 wild-type hosts while levels of CD28 and CD154 were comparable in these two groups (FIG. 2S-V, FIG. 2X). It may be possible that costimulatory requirements of CD4⁺ T lymphocytes may be altered in the absence of CD8⁺ T cells and blockade of CD40-CD154 and CD28/B7 pathways may be insufficient to ameliorate CD4⁺ T cell-mediated rejection (41). To address this, CD27-CD70, ICOS-ICOS Ligand and OX40-OX40 Ligand pathways were inhibited in addition to blocking CD28-B7 and CD40-CD154 in B6 CD8^{-/-} recipients of Balb/c lungs. However, this treatment regimen did not prevent lung allograft rejection when recipients lacked CD8⁺ T cells (FIG. 2Y, FIG. 2Z, FIG. 2AA). These findings directly contrast with previous observations that depletion or deletion of CD8⁺ T cells prolongs survival of skin and heart allografts when recipients are treated with costimulatory blockade (13, 37).

### Example 3. Accepting Lung Allografts are Heavily Infiltrated with Central Memory CD8⁺CD44^{hi}CD62L^{hi}CCR7⁺ T Cells that can Downregulate Alloimmune Responses

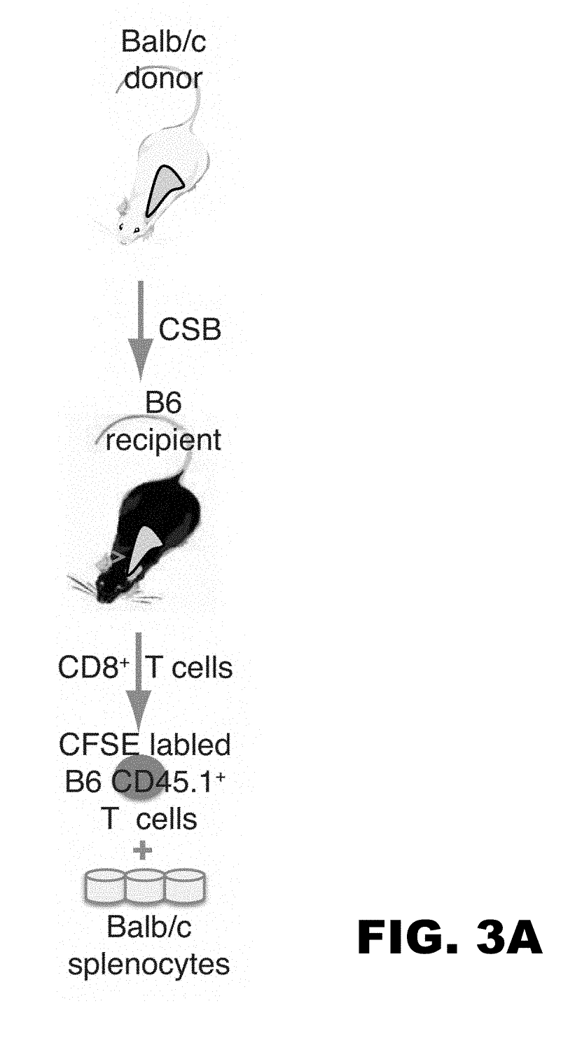

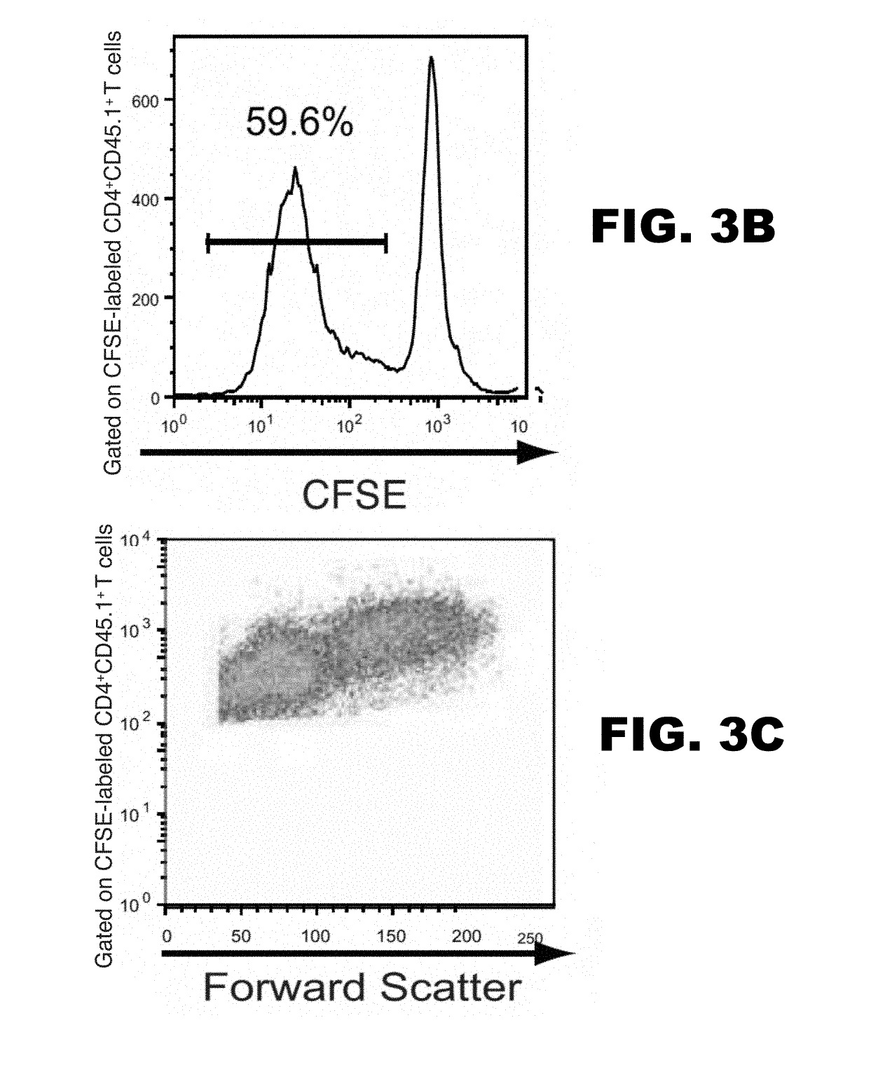

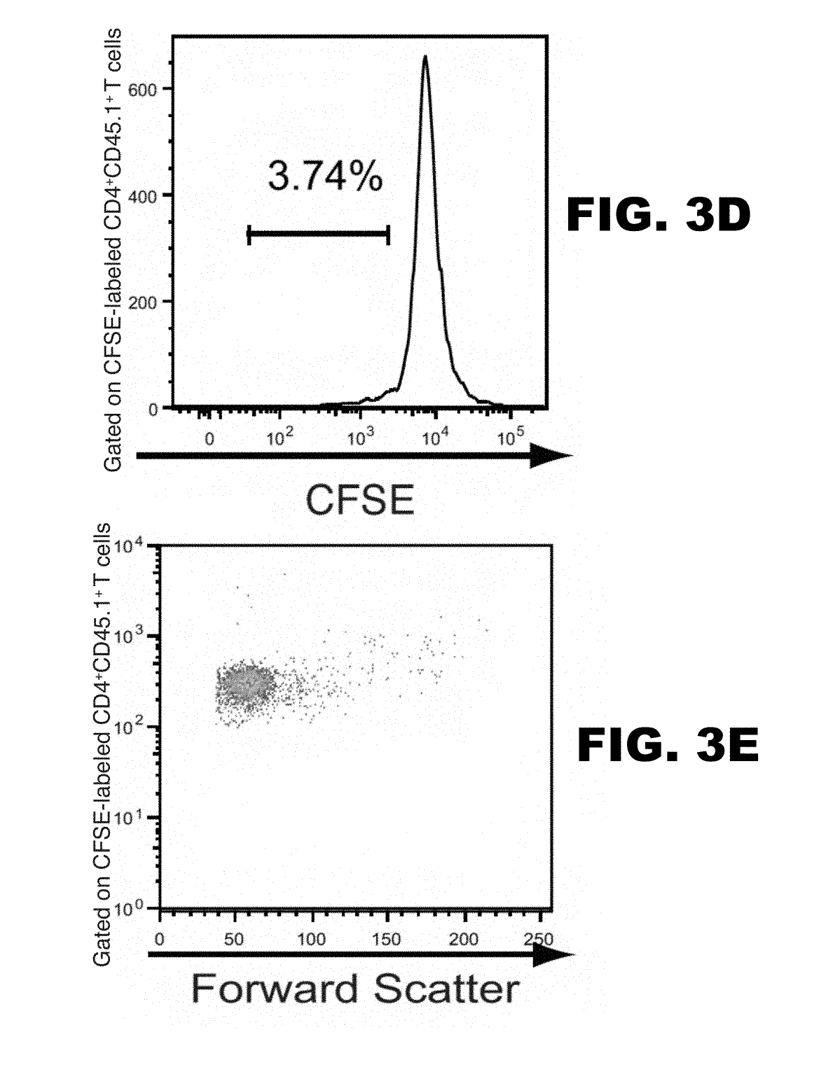

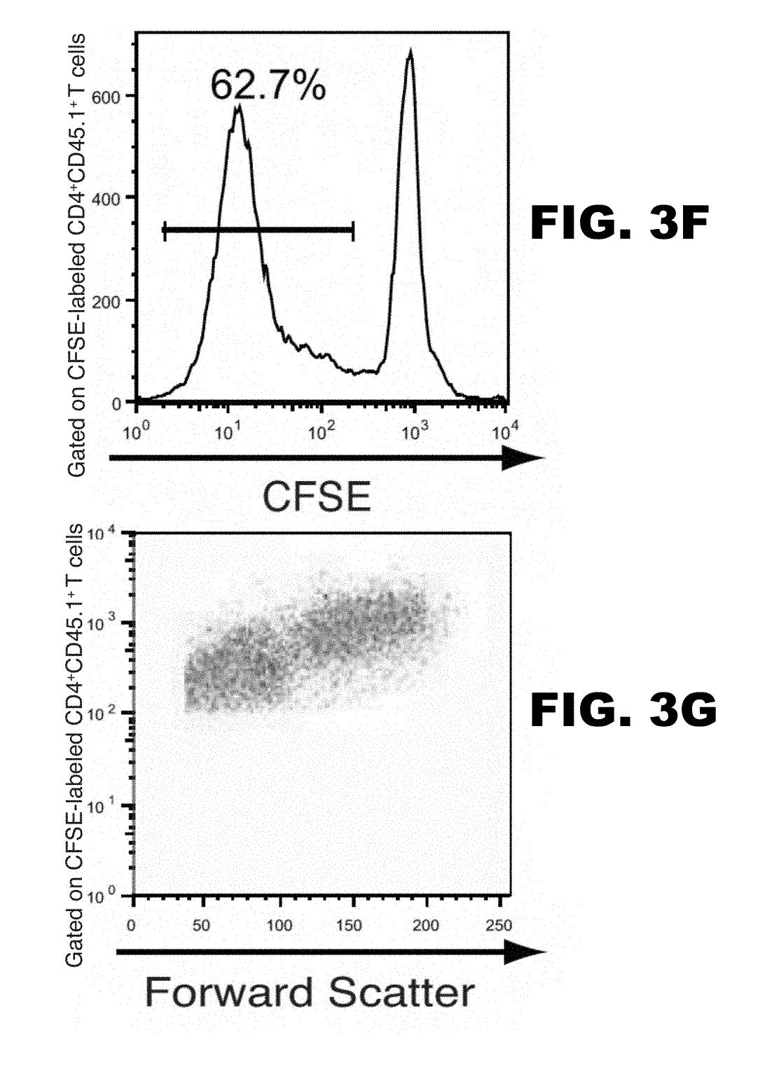

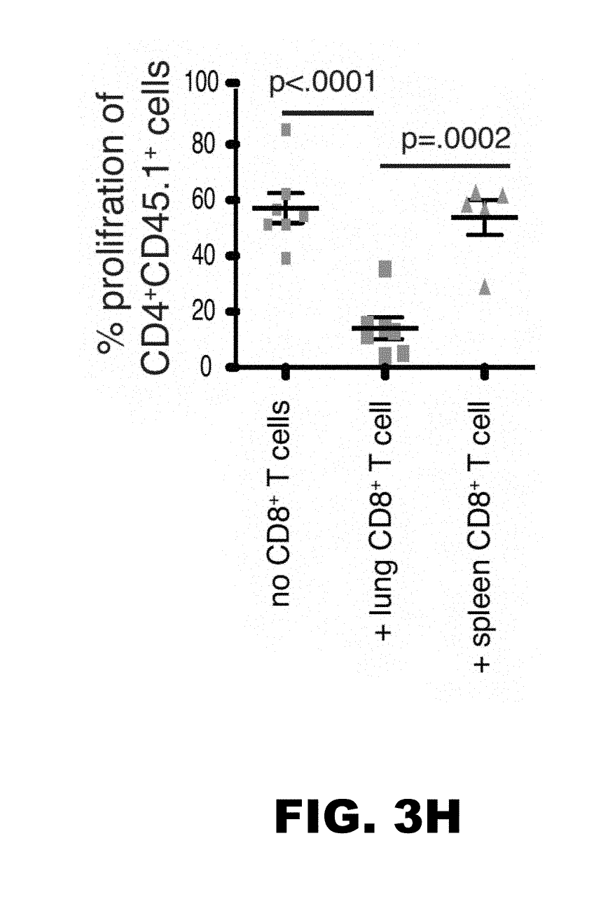

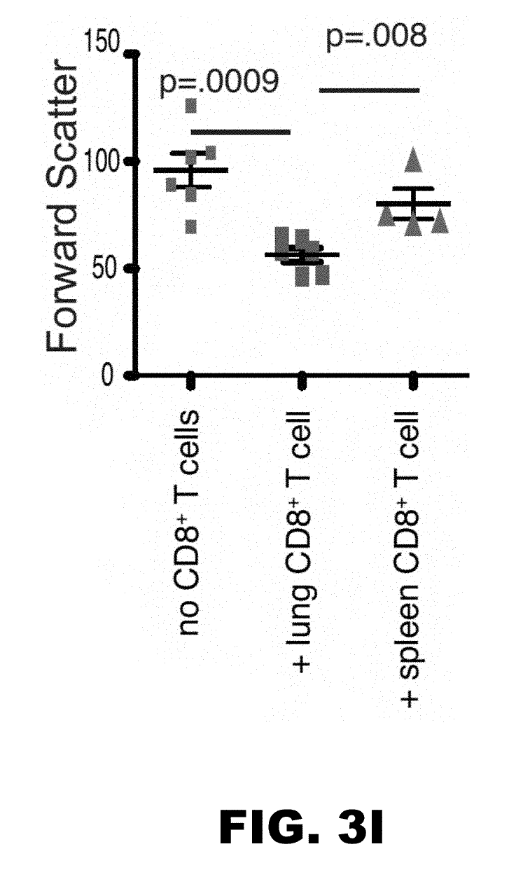

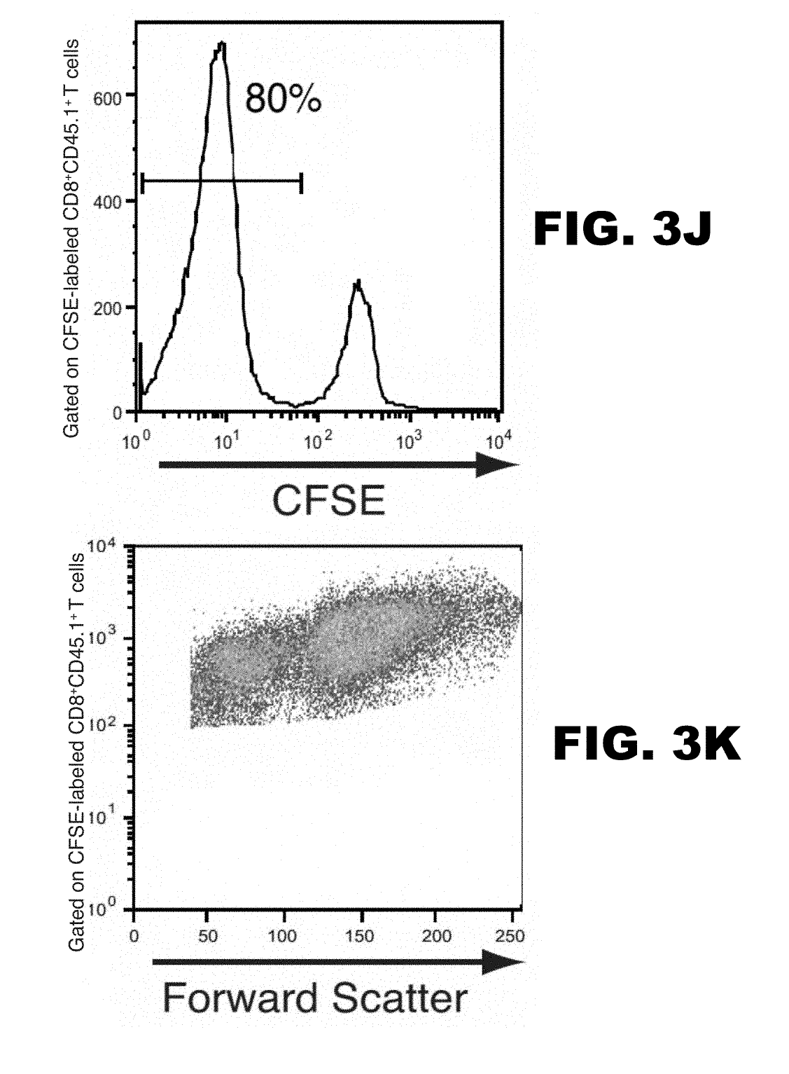

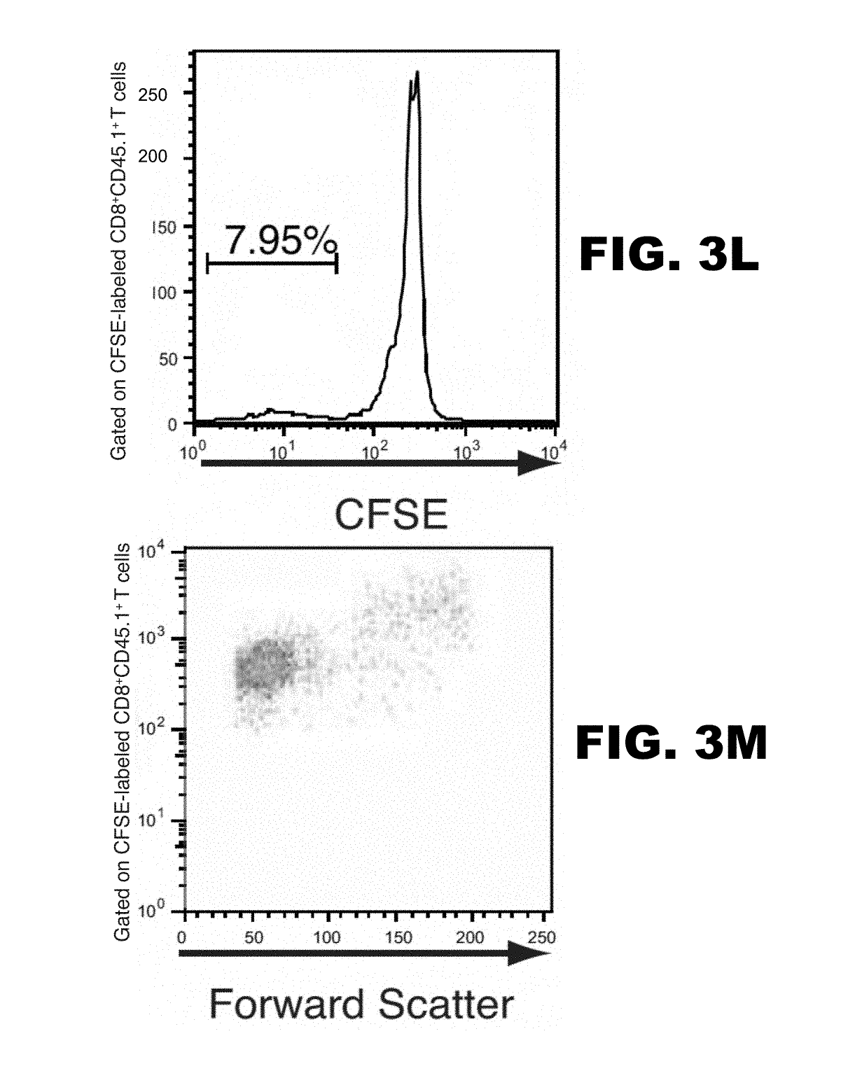

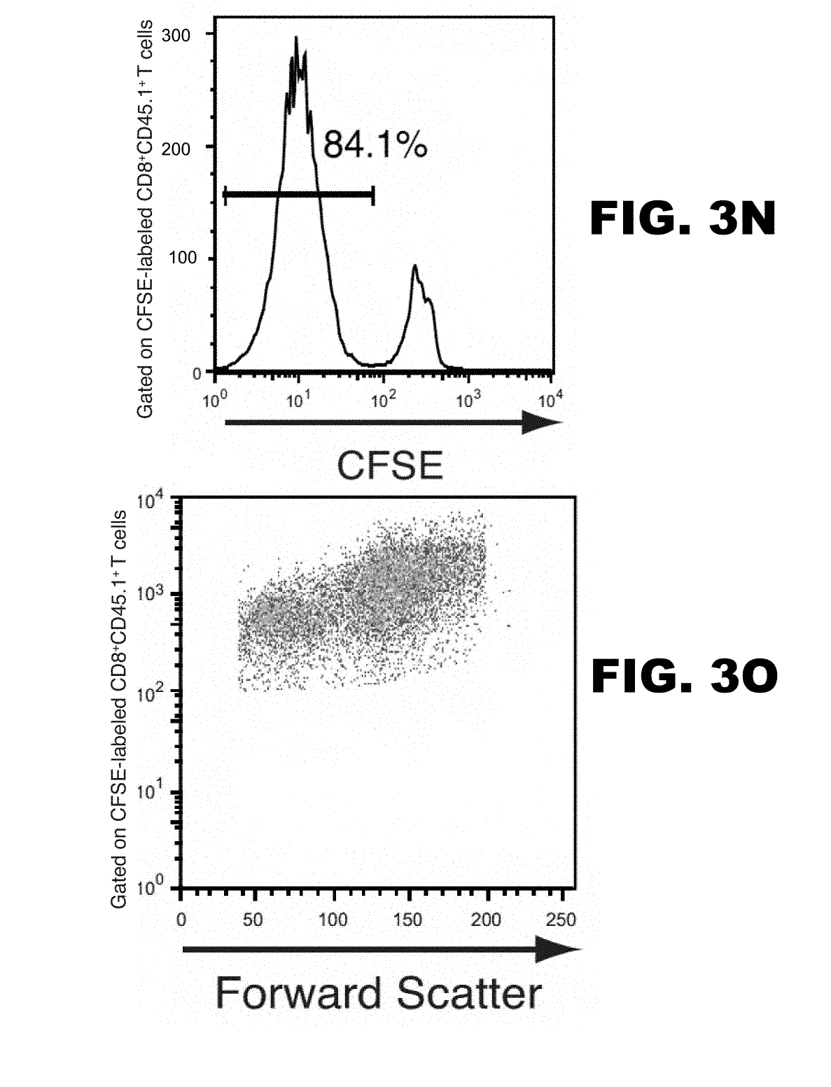

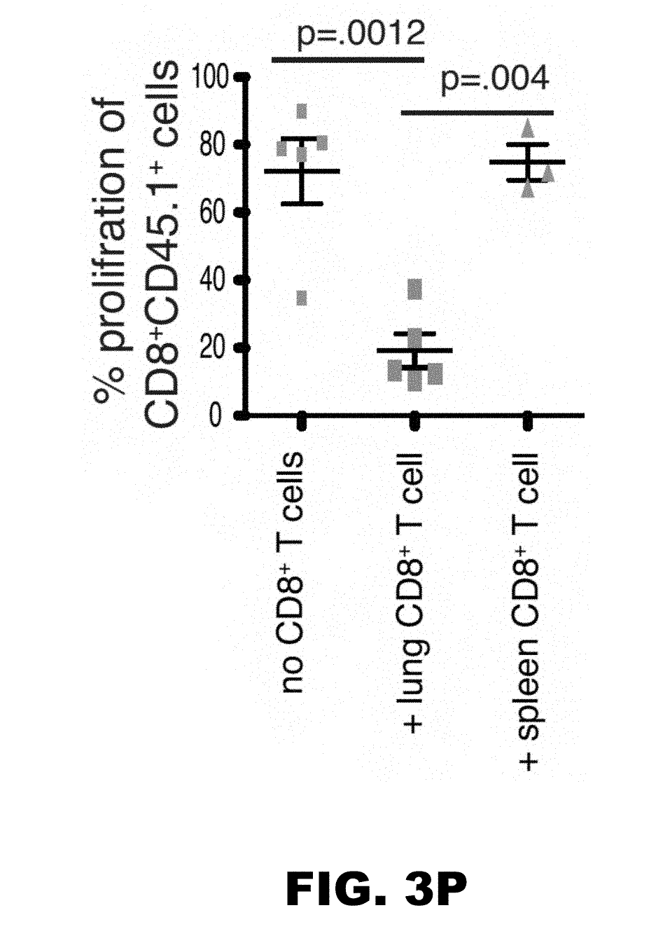

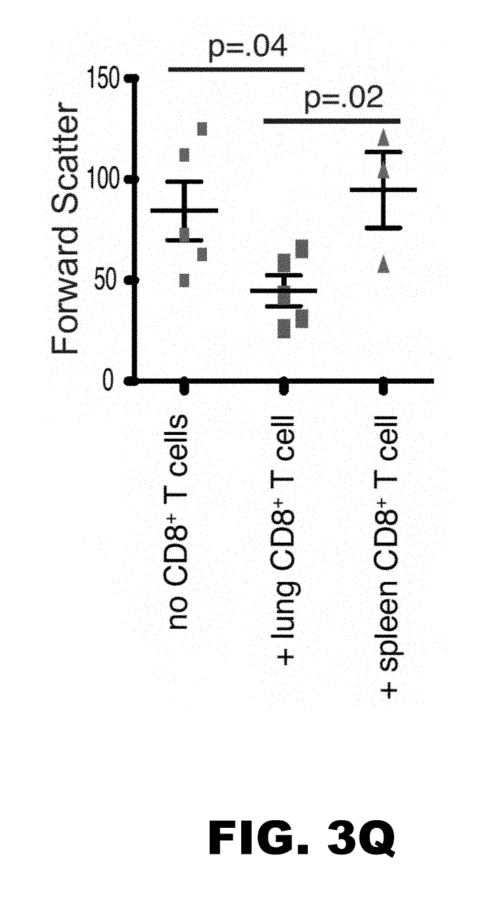

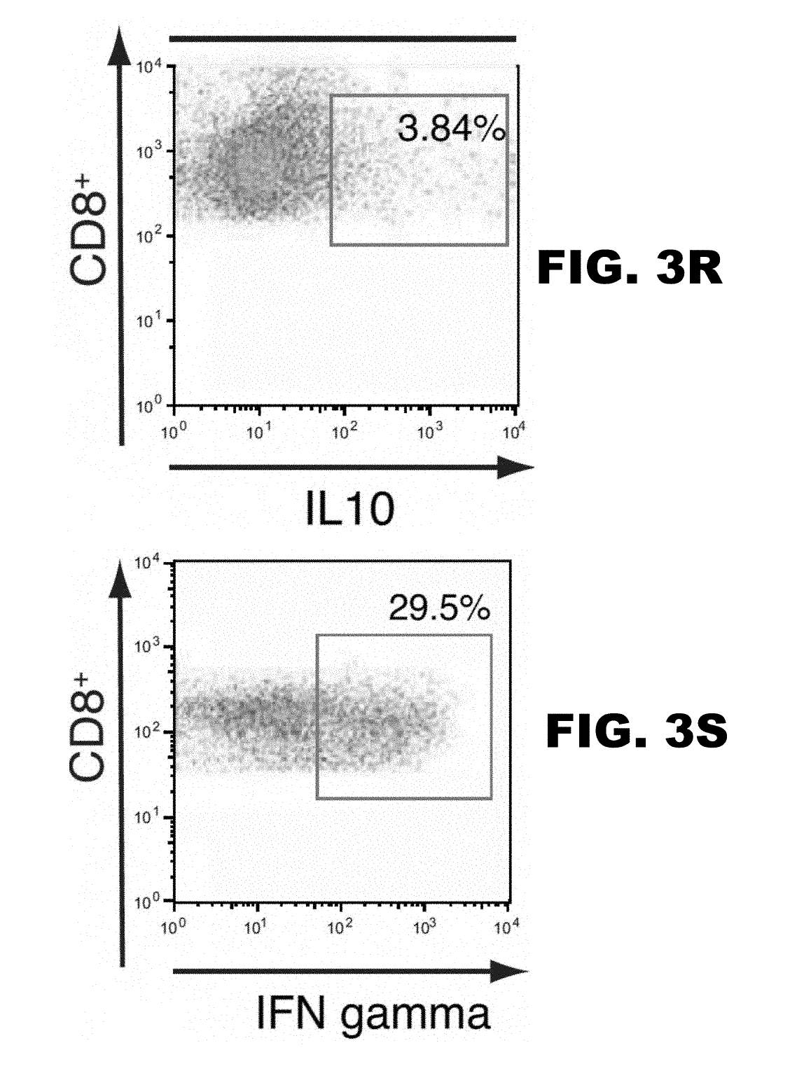

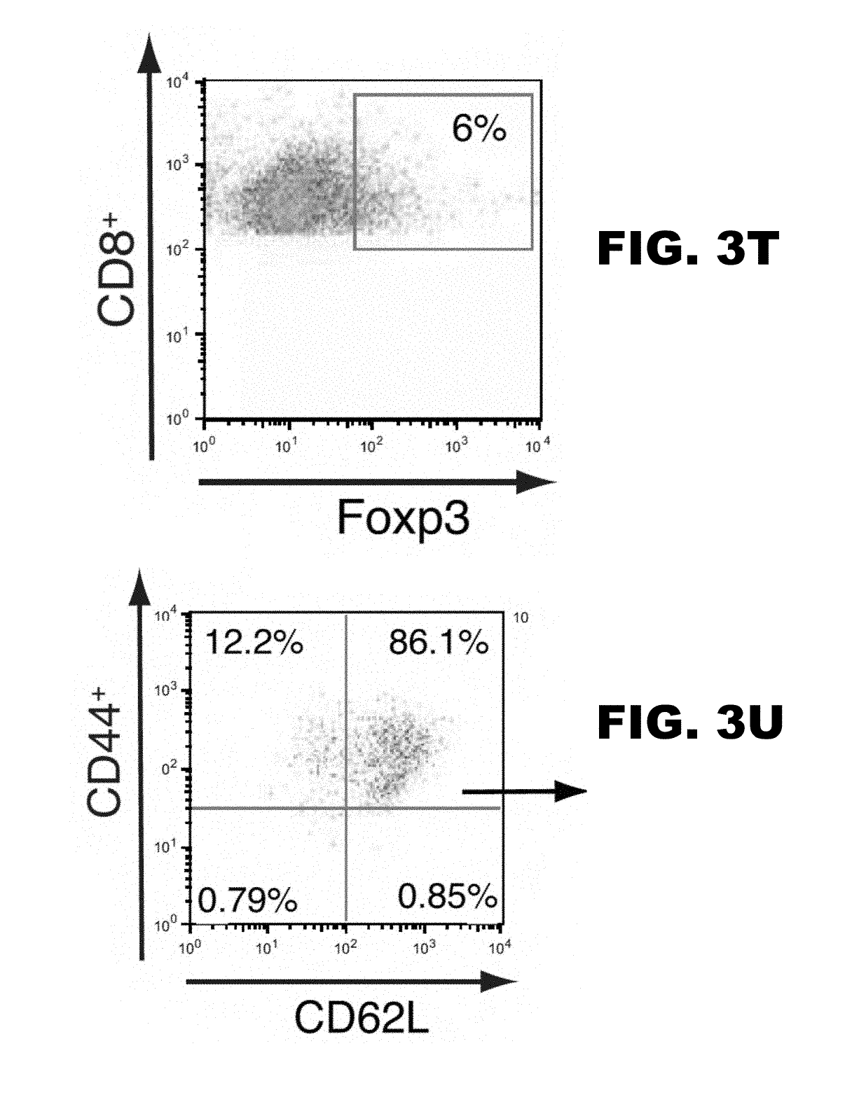

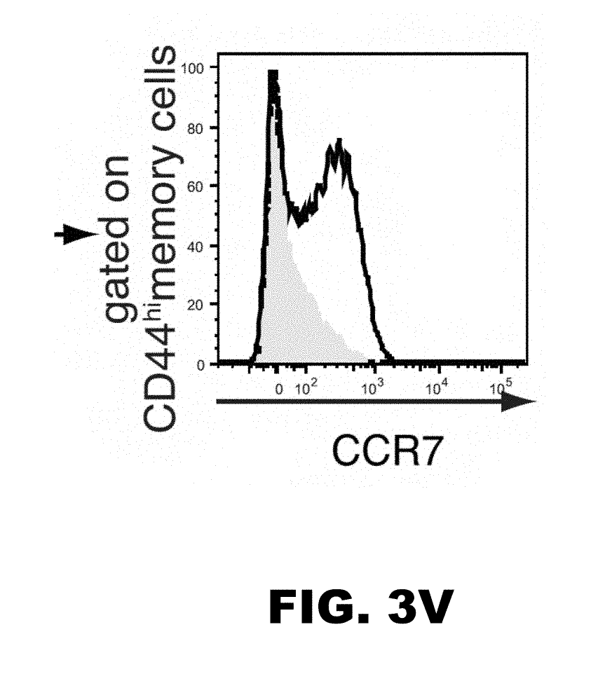

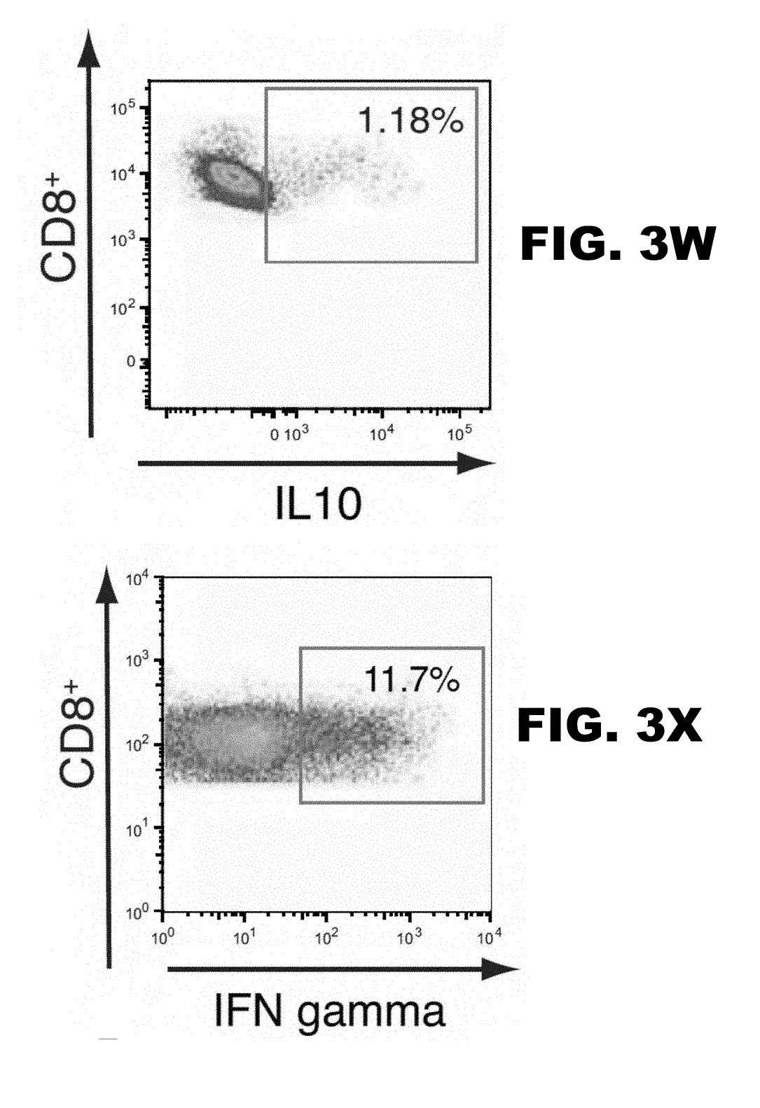

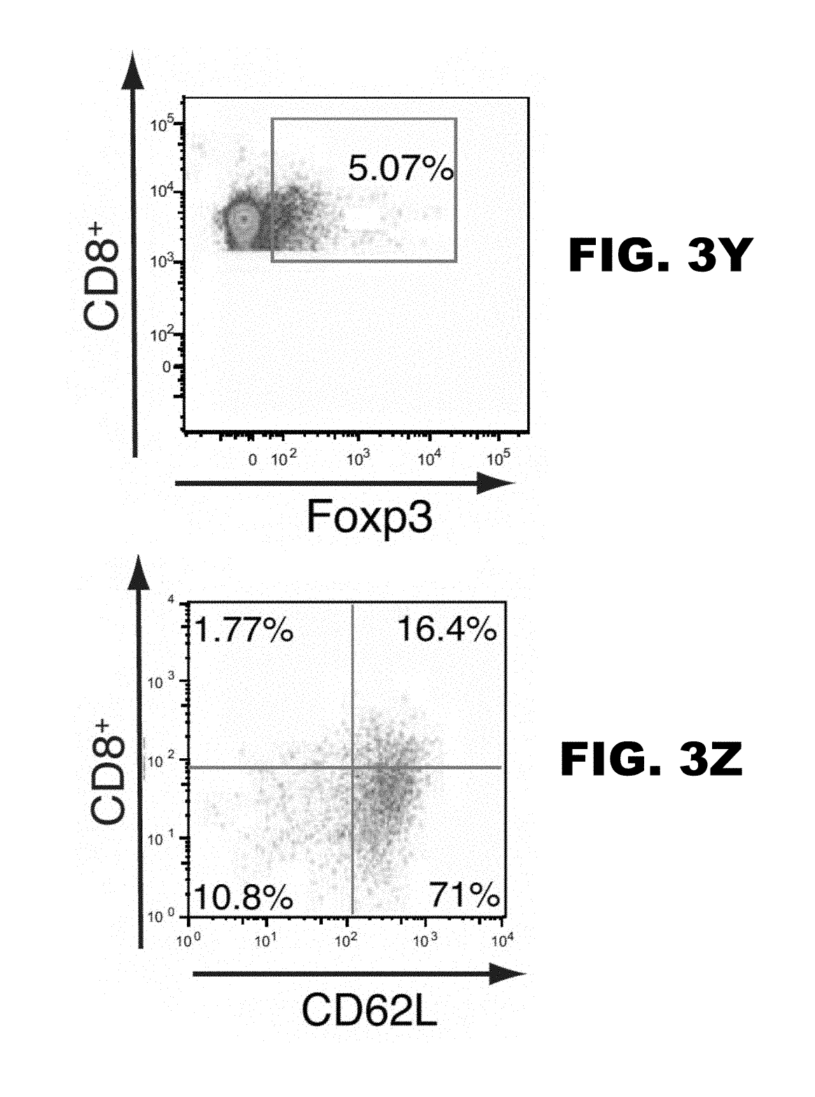

Costimulatory blockade has been described to mediate graft acceptance through the generation of regulatory T lymphocytes (4, 5, 34-38). In order to evaluate if CD8⁺ T lymphocytes with regulatory capacity develop in costimulatory blockade-treated lung recipients, CD8⁺ T cells from the lung grafts and spleens of such mice were isolated and used as “regulators” in in vitro mixed lymphocyte reactions (MLRs) (FIG. 3A). We found that CD8⁺ T lymphocytes isolated from accepting Balb/c→B6 lung allografts, but not spleens of these recipients, inhibited proliferation and blasting of B6 CD45.1⁺ CD4⁺ (FIG. 3B-I) and B6 CD45.1⁺ CD8⁺ T lymphocytes (FIG. 3J-0) when stimulated with Balb/c splenocytes. These findings suggested that CD8⁺ T cells with regulatory capacity accumulate in accepting lung allografts. While described to have regulatory function in other models (42-44) very few CD8⁺IL-10⁺ or CD8⁺Foxp3⁺ cells were detectable in accepting lung allografts (FIG. 3R-V). Notably, however, a large portion of CD8⁺ T cells in accepting grafts had the capacity to produce IFN-γ and expressed phenotypic markers consistent with central memory T lymphocytes (CD44^{hi}CD62L^{hi}CCR7⁺) (45). By contrast, most CD8⁺ T cells in the spleens of graft-accepting recipients were naïve (CD44^{low}CD62L^{hi}) with lower levels of IFN-γ production (FIG. 3W-Z).

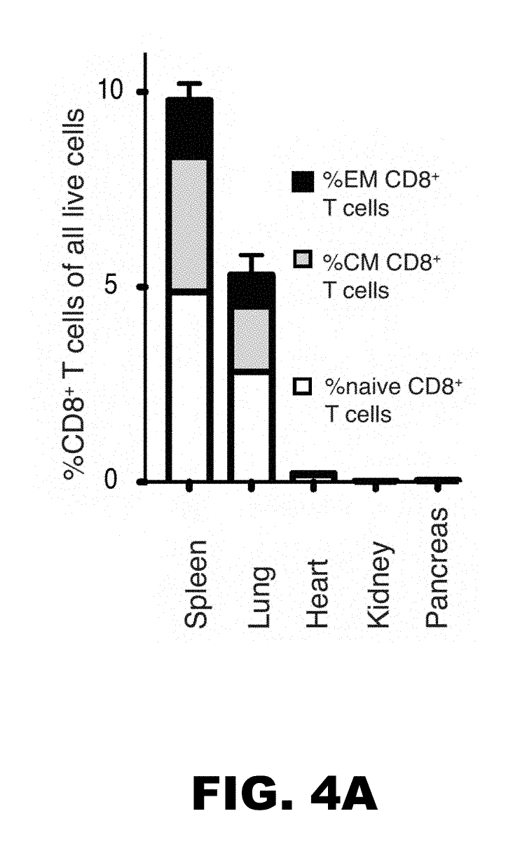

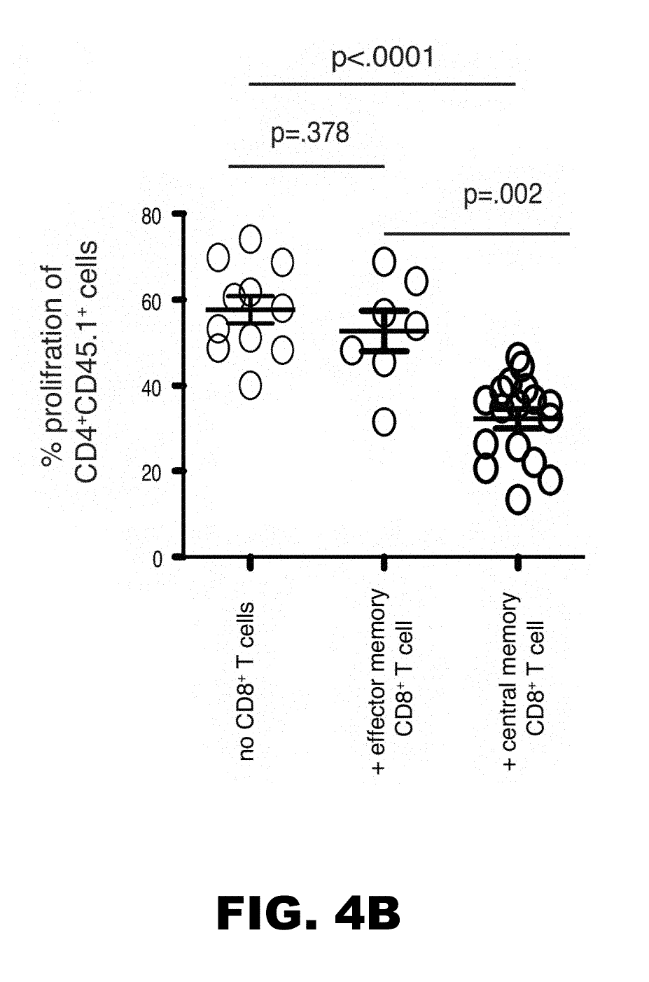

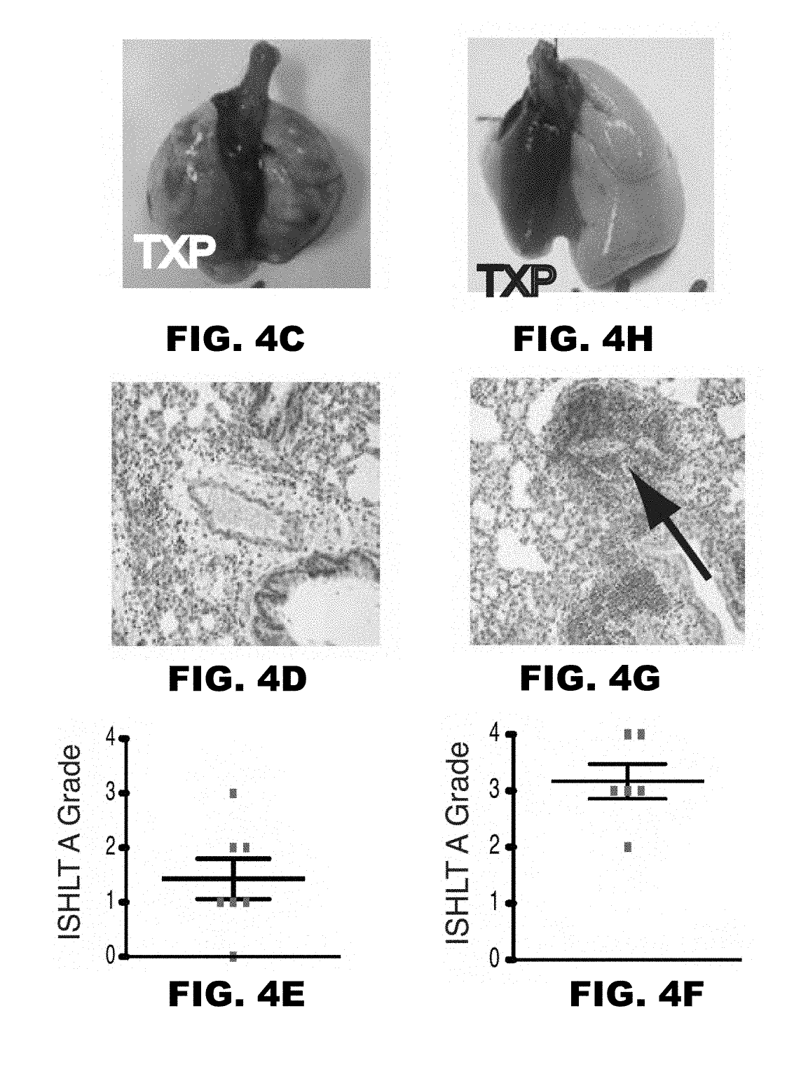

While the vast majority of studies suggest that memory T lymphocytes potentiate alloimmune responses and inhibit tolerance induction (46, 47), it is possible that certain subsets of these cells may suppress alloreactivity (48). It is noteworthy that CD8⁺ T lymphocytes, including memory CD8⁺ T cells, are present in the lung at baseline, even in the absence of acute inflammation or alloantigen stimulation (FIG. 4A). This has been attributed to the lung's constant exposure to the environment and need to mount rapid responses to pathogens (49). It was next investigated if memory CD8⁺ T cells from lungs of resting mice also had regulatory capacity. For this purpose, CD8+CD44^{hi}CD62L^{hi} central memory and CD8+CD44^{hi}CD62L^{low} effector memory T lymphocytes (45) from resting B6 mice were flow cytometrically sorted and used as regulators in in vitro MLRs similar to methods described above (FIG. 3A). Interestingly, even without prior in vitro stimulation central memory CD8⁺ T lymphocytes could suppress proliferation of B6 CD45.1⁺ CD4⁺ T cells stimulated with Balb/c splenocytes (FIG. 4B), albeit to a lesser extent than those derived from transplanted grafts (FIG. 3B-I). Freshly isolated CD8⁺ effector memory T lymphocytes, however, had no effect on B6 CD45.1⁺ CD4⁺ T cell proliferation (FIG. 4B). To further evaluate if different subsets of memory T cells could influence the alloimmune response, central memory or effector memory CD8⁺ T cells were generated in vitro by culturing B6 splenocytes with irradiated Balb/c stimulators in the presence of IL-15 or IL-2 respectively as previously described (50, 51). B6 CD8^{-/-} mice, reconstituted with central memory CD8⁺ T cells accepted, while those reconstituted with effector memory CD8⁺ T cells rejected Balb/c lung allografts after costimulatory blockade (FIG. 4C-H). Collectively, these data demonstrate that subtypes of memory CD8⁺ T cells can differentially influence the alloimmune response and that central memory CD8⁺ T cells play a critical role in lung allograft acceptance.

### Example 4. Central Memory CD8⁺ T Cells Suppress Alloimmune Responses Through IFN-γ-Mediated Production of Nitric Oxide

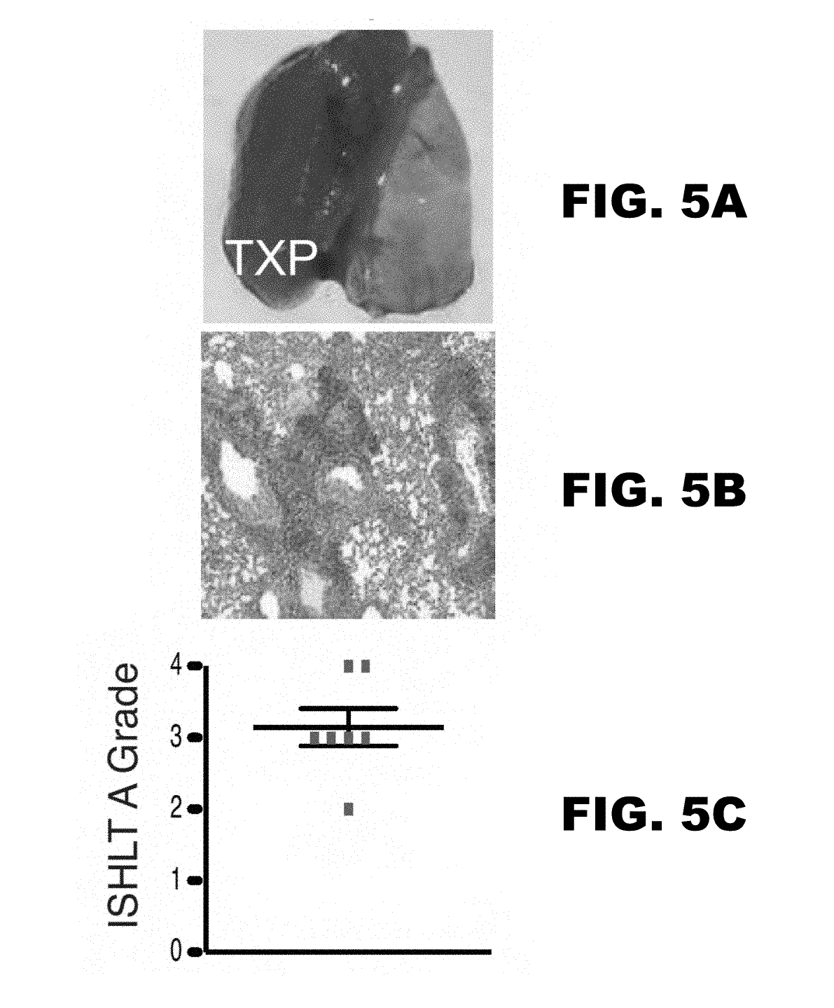

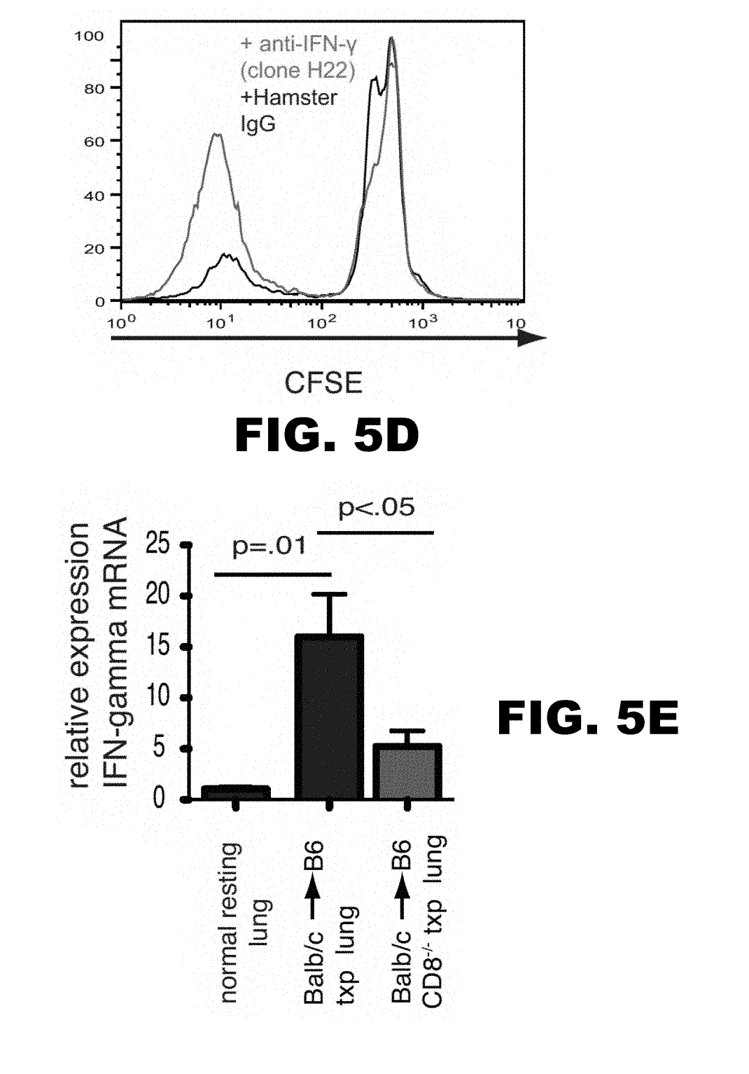

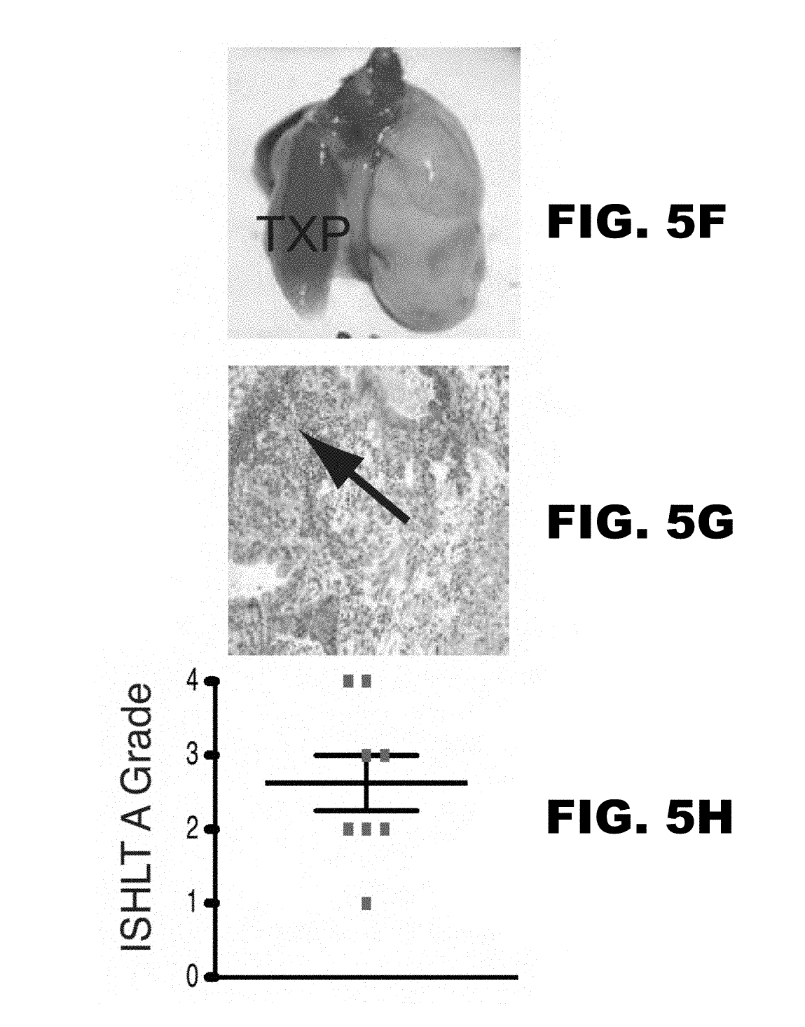

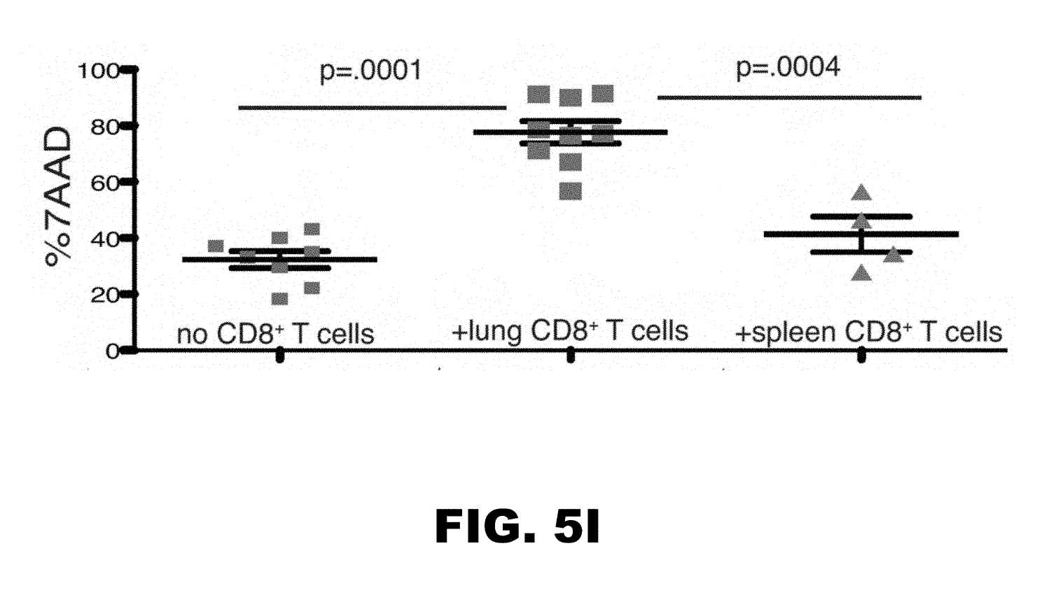

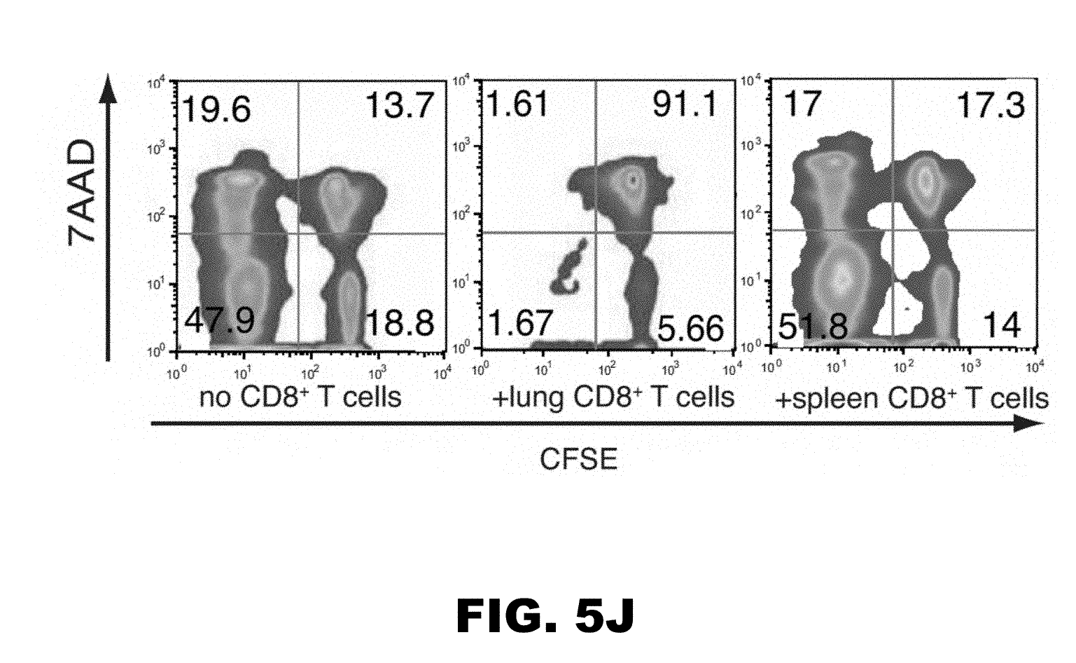

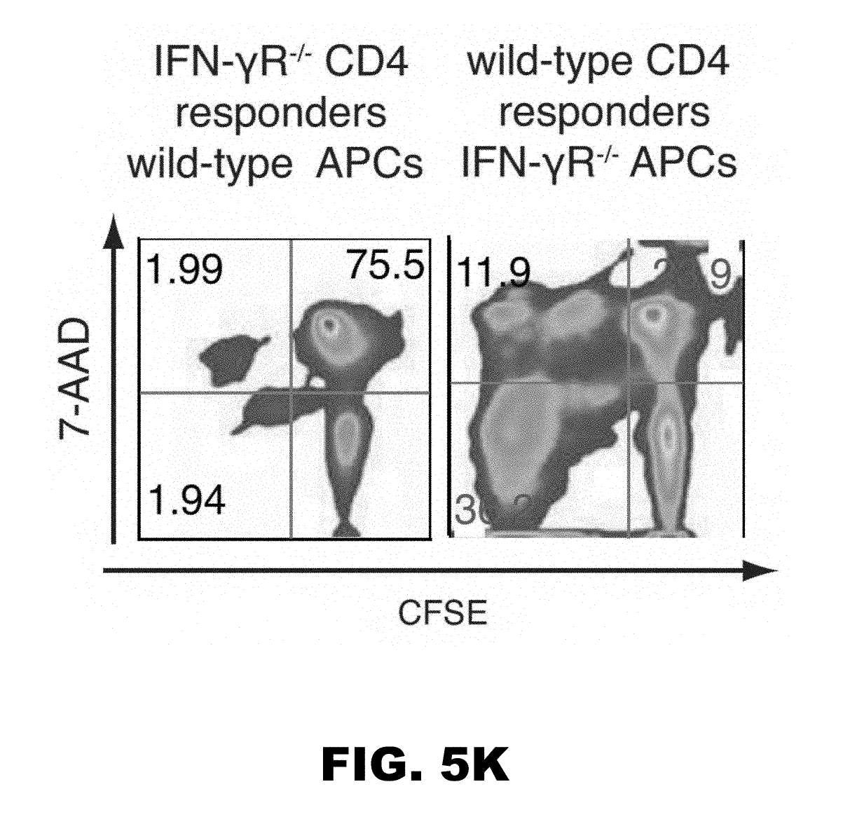

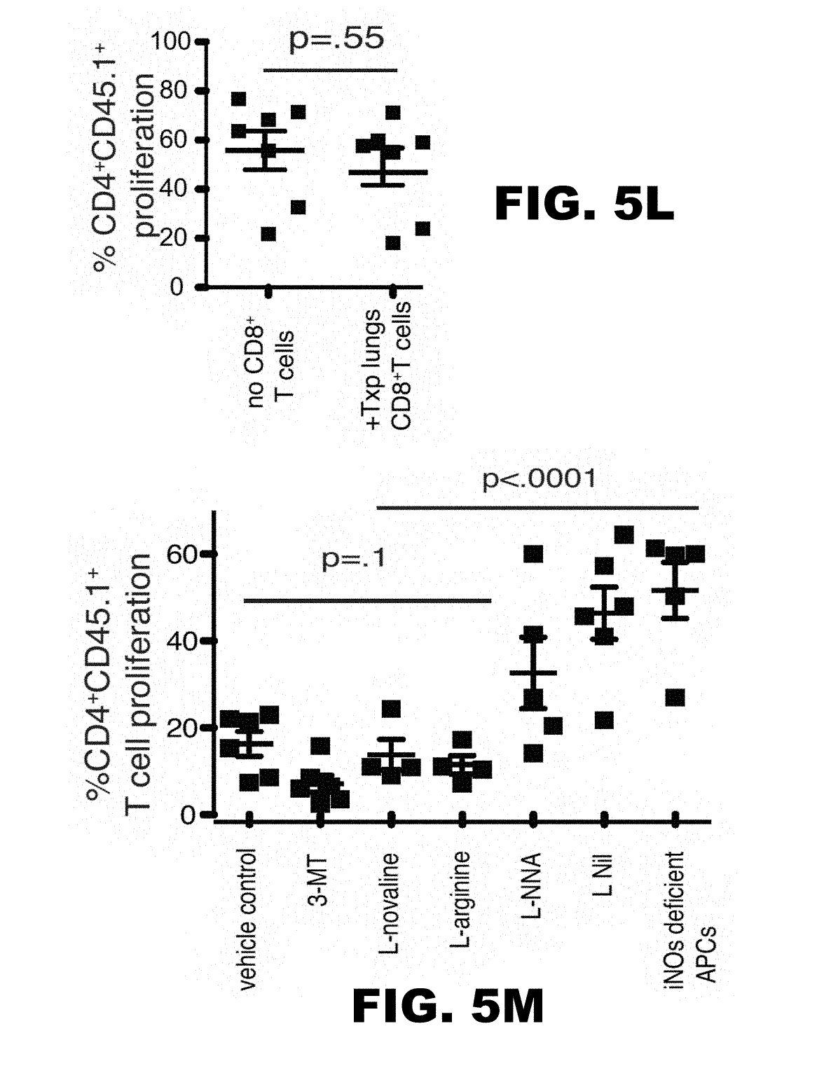

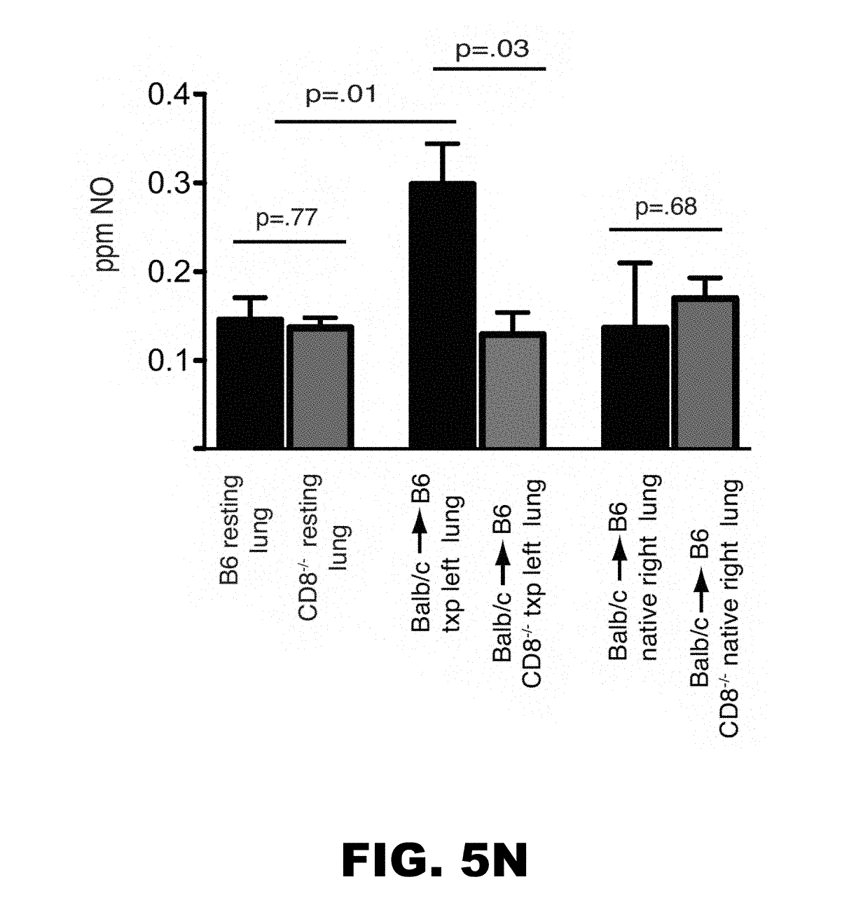

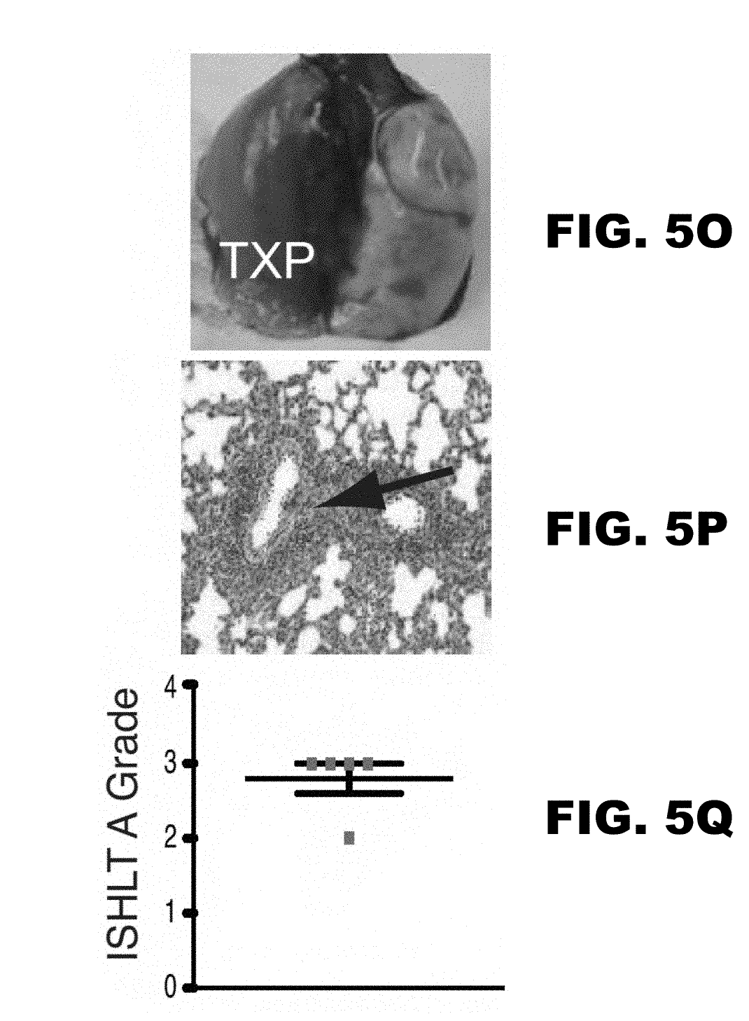

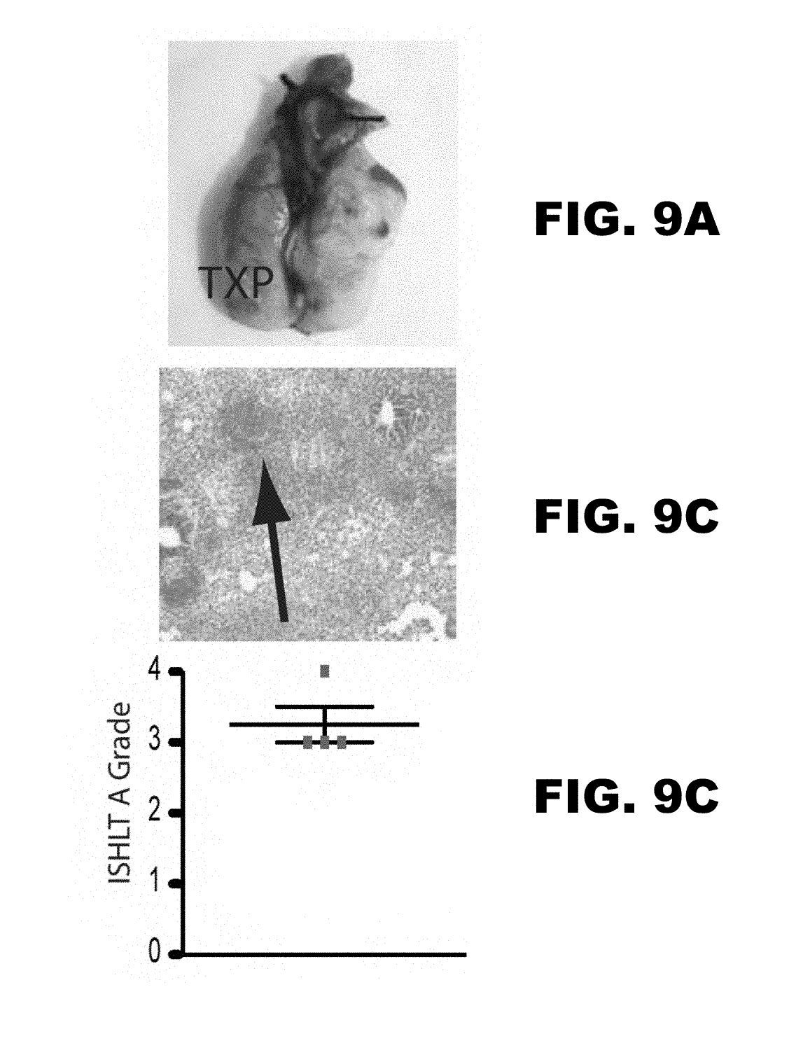

Since central memory T cells are known to be rapid producers of pro-inflammatory cytokines, it was next examined whether CD8⁺ T lymphocytes mediate lung allograft acceptance through secretion of TNF-α or IFN-γ. Balb/c lungs were accepted by TNF-α-deficient B6 recipients or B6 CD8^{-/-} mice that were reconstituted with TNF-α-deficient B6 CD8⁺ T cells (data not shown). By contrast, allograft acceptance was abrogated if recipient mice were pretreated with IFN-γ-neutralizing antibody or IFNγ^{-/-} animals were used as hosts (FIG. 5A-C and FIG. 9A-C). CD8⁺ T cell-mediated suppression of CD4⁺ T cell proliferation was also abrogated in vitro in the presence of IFN-γ neutralizing antibody (FIG. 5D). IFN-γ levels were significantly elevated in grafts after transplantation of Balb/c lungs into immunosuppressed B6 wild-type compared to B6 CD8^{-/-} recipients (FIG. 5E). Finally, injection of IFN-γ^{-/-} CD8⁺ T cells into CD8^{-/-} mice failed to rescue Balb/c lung allografts from rejection despite costimulatory blockade (FIG. 5F-H). Taken together, these data demonstrate that IFN-γ production by CD8⁺ T cells plays a critical role in lung allograft acceptance.

In in vitro mixed lymphocyte reactions described above (FIG. 3A), it was noted that the majority of CD4⁺CD45.1⁺ responder T lymphocytes were not viable as measured by 7-AAD uptake when CD8⁺ T cells obtained from accepting lung allografts were added to the cultures (FIG. 5I, FIG. 5J). Moreover, sensitivity of antigen presenting cells to IFN-γ was critical for CD8⁺ T cell-mediated suppression as proliferation of IFN-γ receptor-deficient CD4⁺ T cells was inhibited by allograft-derived CD8⁺ T cells, but no inhibition was evident if IFN-γ receptor-deficient antigen presenting cells were used (FIG. 5K). Also, CD8⁺ T cell-mediated suppression was not observed when T cells were activated with anti-CD3 and anti-CD28 antibodies in an antigen presenting cell-free system (FIG. 5L). Taken together, these data indicate that CD8⁺ T cells require antigen presenting cells to mediate the downregulation of T lymphocyte responses.

Since metabolism of essential amino acids is a common mechanism of immunoregulation by antigen presenting cells (52), various pharmacologic inhibitors were added to in vitro mixed lymphocyte reactions and it was noted that only L-NNA (NG-nitro-L-Arginine; L-NG-Nitroarginine), an inhibitor of endothelial, neuronal, and inducible nitric oxide synthase (eNOS, nNOS, and iNOS, respectively), and L-nil (N6-(1-iminoethyl)-L-lysine, dihydrochloride), a selective iNOS inhibitor, were able to attenuate CD8⁺ T cell-mediated suppression of CD4⁺ T lymphocyte proliferation (FIG. 5M). Similarly, iNOS-deficient stimulators also prevented CD8⁺ T cell-mediated suppression of CD4⁺ T lymphocyte proliferation (FIG. 5M). L-novaline and 1-methyl-tryptophan (1-MT), selective inhibitors of arginase and indoleamine-pyrrole 2,3-dioxygenase (IDO), respectively, did not affect proliferation of CD4⁺ T cells. Furthermore, the addition of L-arginine did not reverse CD8⁺ T cell-mediated suppression suggesting that amino acid depletion was not likely to be the principal method of immunoregulation (FIG. 5M). This observation is consistent with the known role of IFN-γ in inducing iNOS expression (53).

Based on the finding that iNOS is critical to mediate suppression by CD8⁺ T cells and reports showing that local production of NO can downregulate immune responses by limiting proliferation and survival of T lymphocytes (54), NO production in lungs was directly measured. A near doubling in NO levels was evident in accepting Balb/c→B6 lung grafts compared to unmanipulated lungs of resting mice (FIG. 5N). By contrast, increases in NO levels in Balb/c→B6 CD8^{-/-} grafts were not observed. NO levels in the right native lungs of Balb/c→B6 and Balb/c→B6 CD8^{-/-} transplant recipients were comparable to resting lungs. To further examine the role of NO in graft acceptance, Balb/c lungs were transplanted into costimulatory blockade-treated recipient B6 mice deficient in iNOS and it was observed that these grafts were rejected (FIG. 5O-Q). Thus, lung transplant acceptance is dependent on NO production by graft-infiltrating recipient cells.

### Example 5. Gαᵢ-Coupled Chemokine Receptors Regulate Trafficking of Alloantigen-Specific CD8⁺ Central Memory T Cells into Lung Allografts

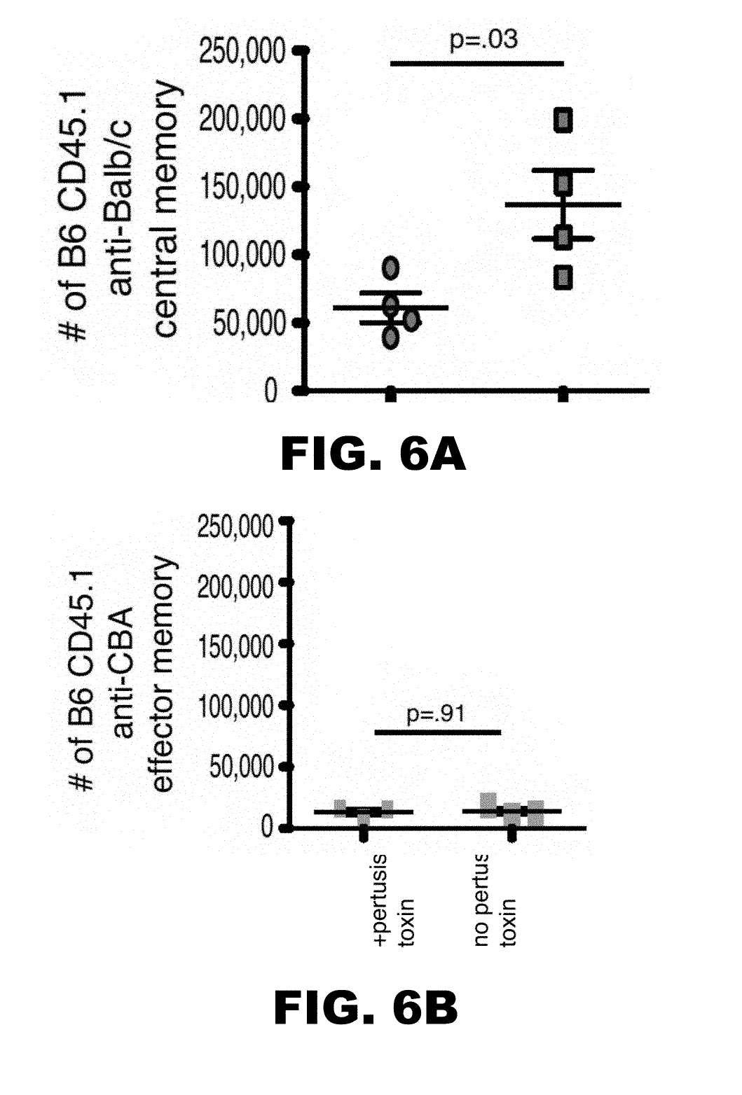

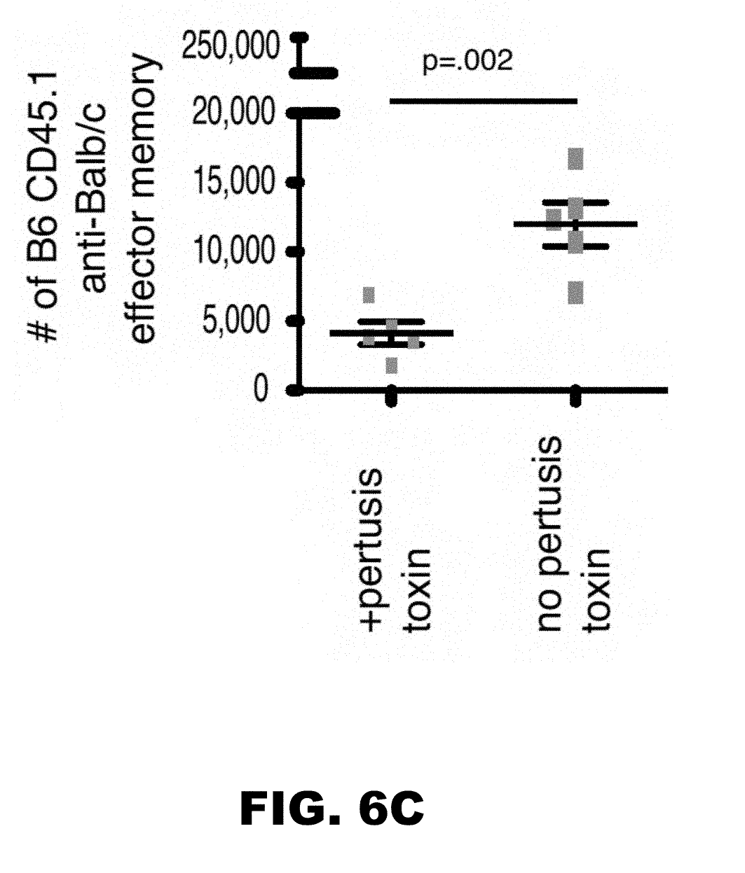

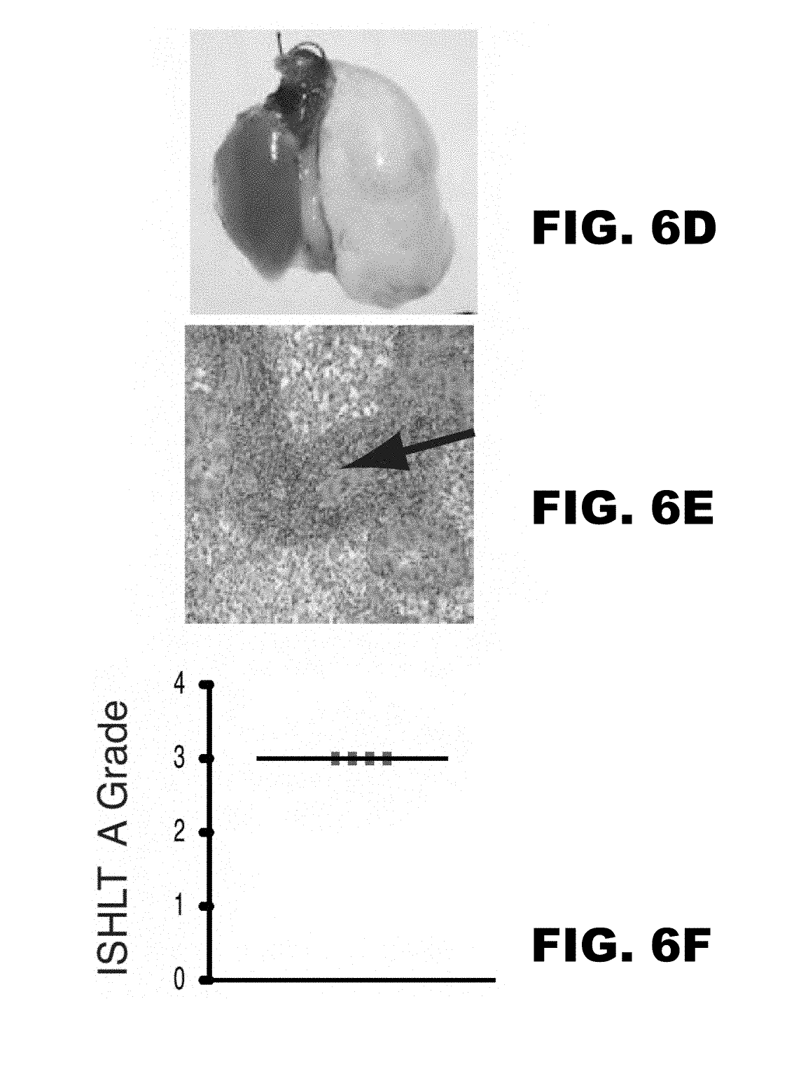

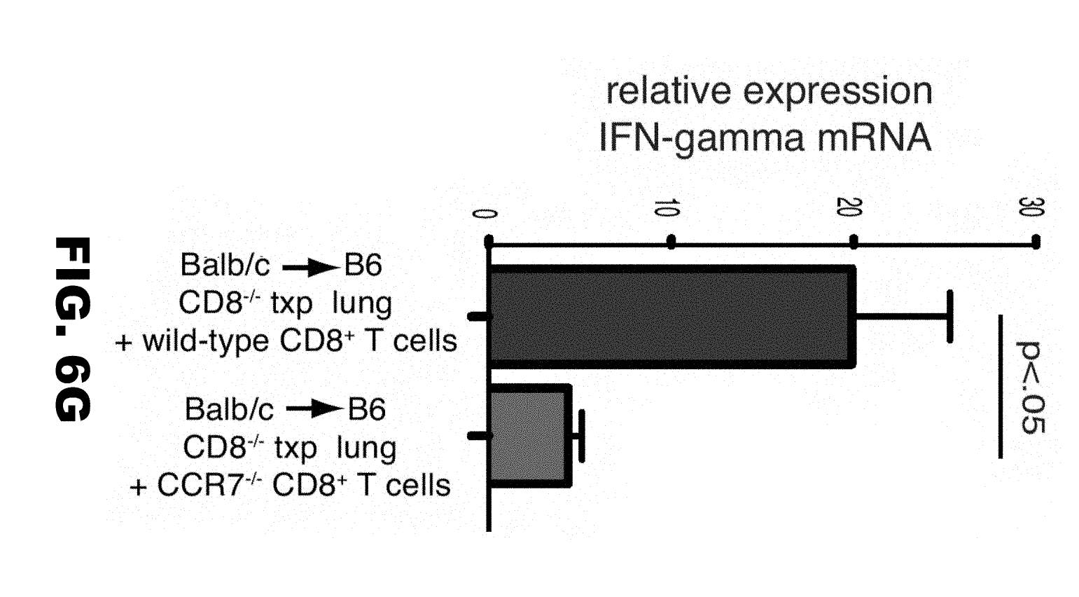

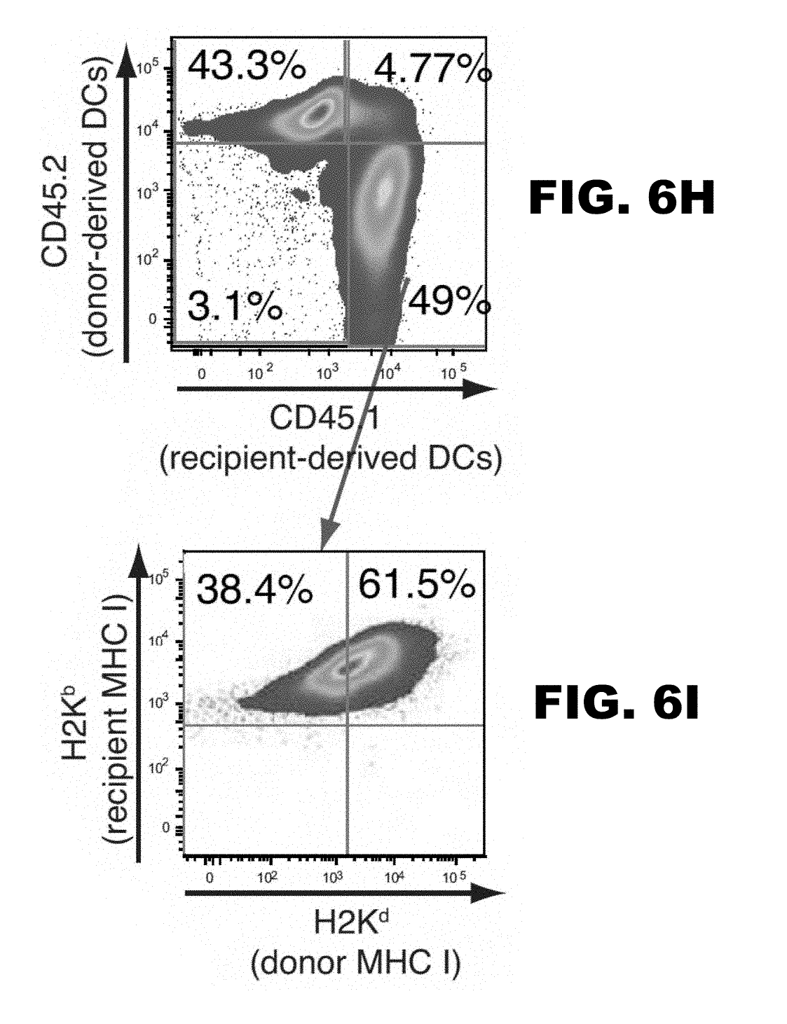

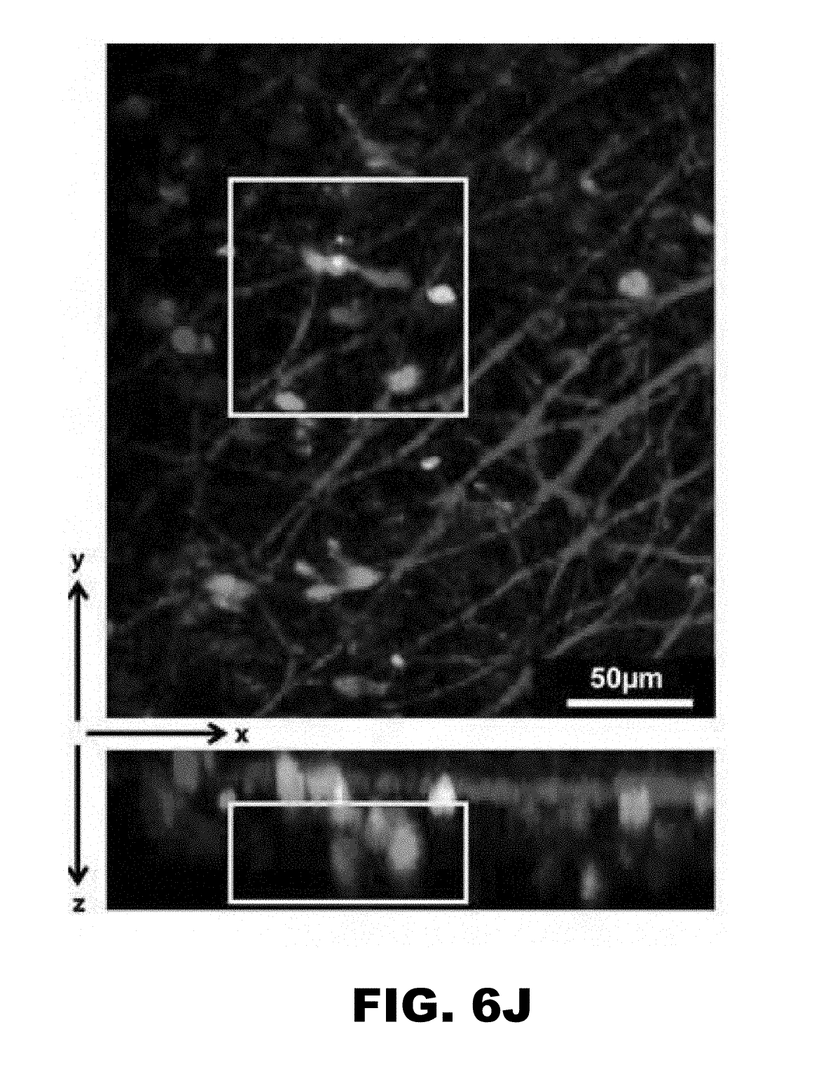

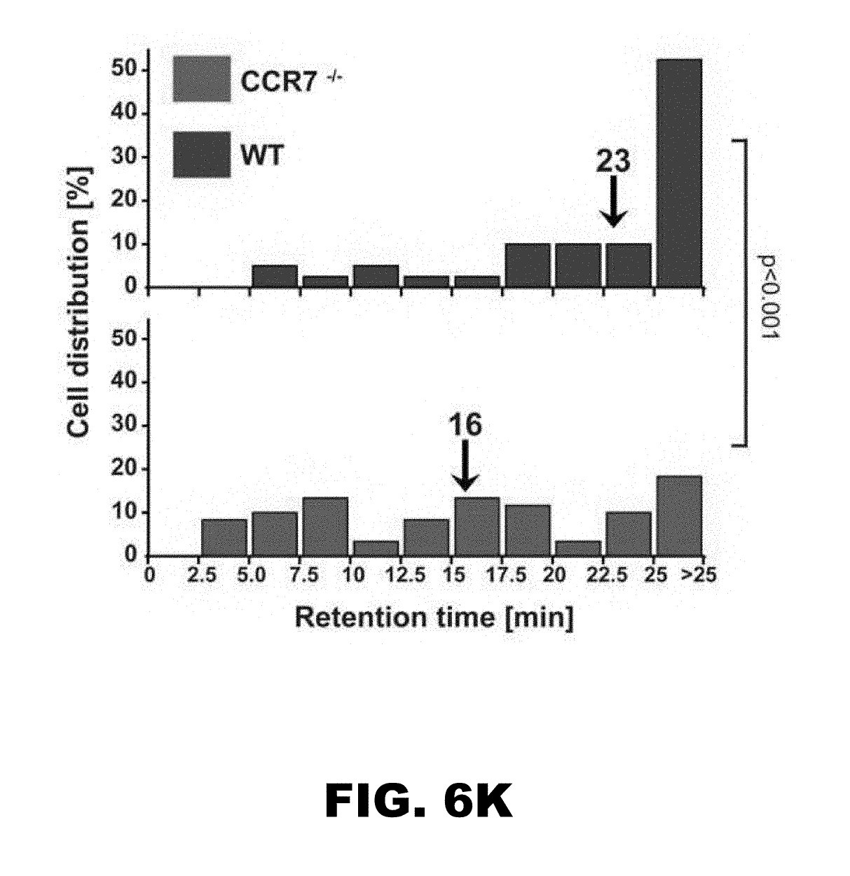

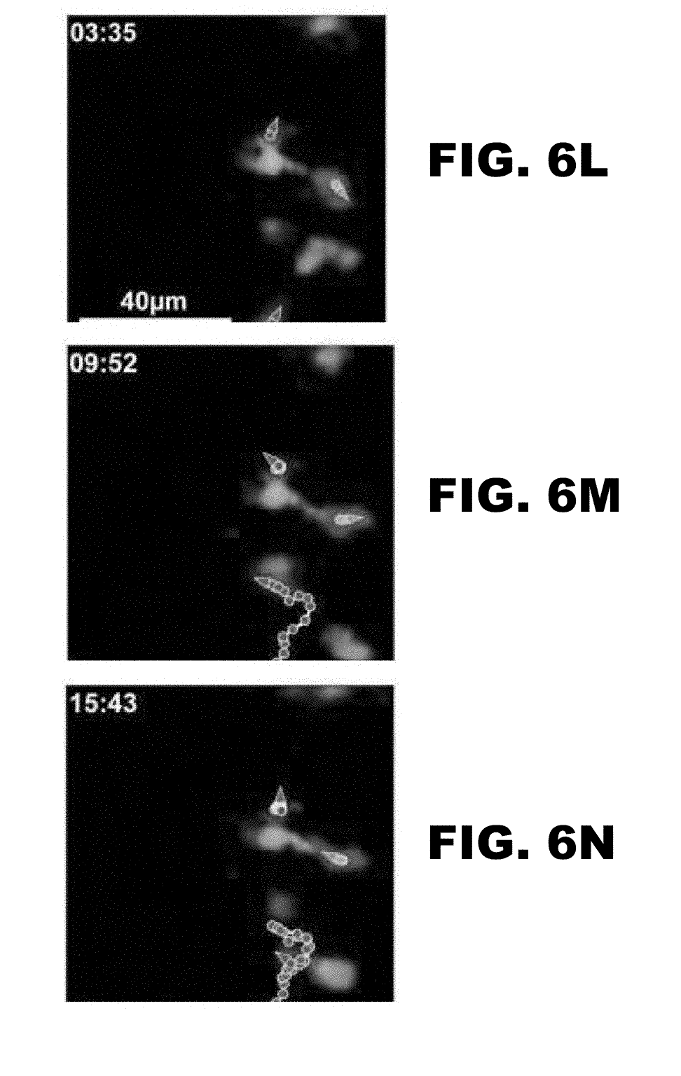

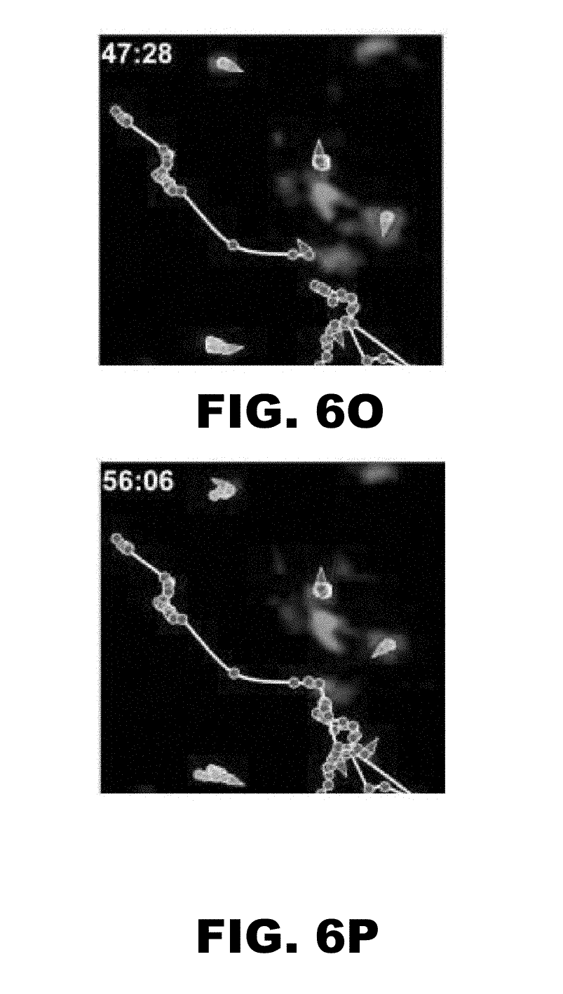

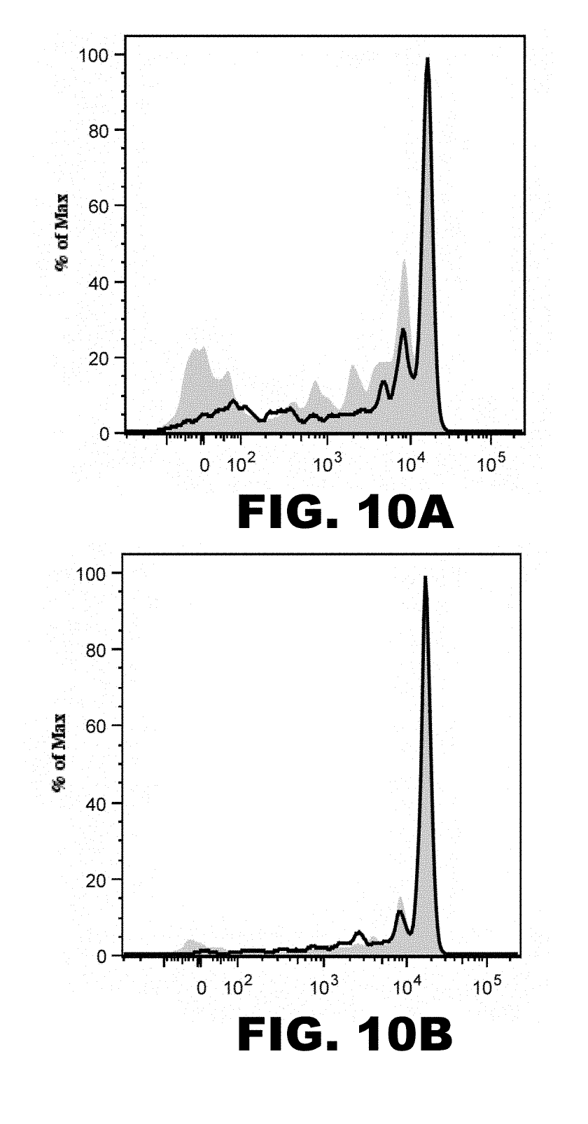

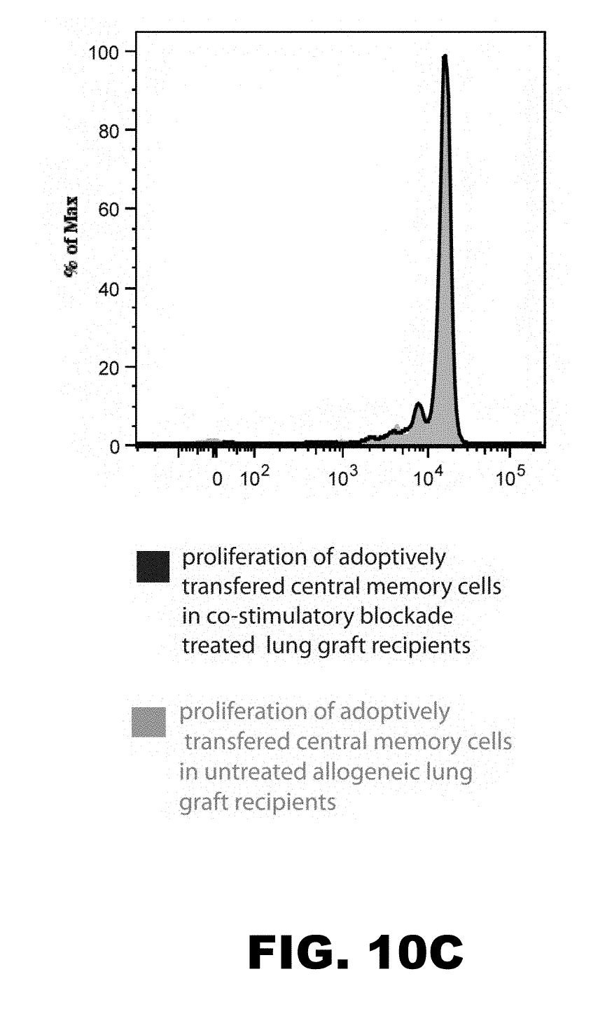

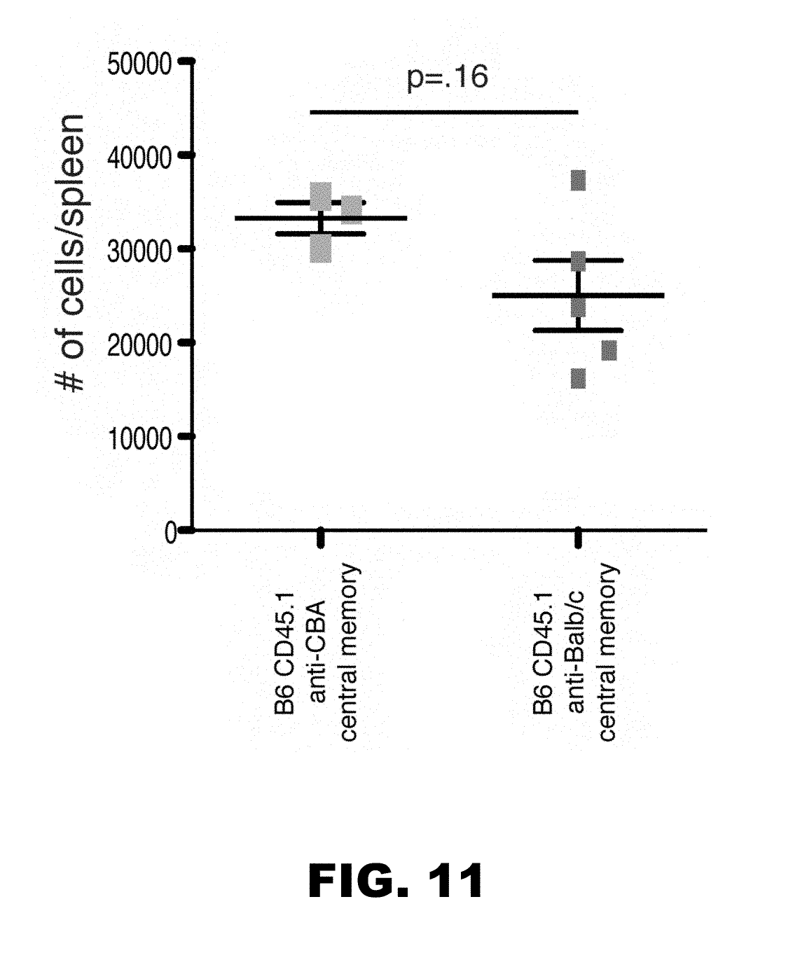

Next, the behavior of CD8⁺ central memory T cells in immunosuppressed lung graft recipients was characterized. It was observed that central memory CD8⁺ T lymphocytes that infiltrated grafts in costimulatory blockade-treated hosts have undergone proliferation, albeit to a lesser degree than in non-immunosuppressed recipients (FIG. 10A-C). To investigate their trafficking requirements, donor-specific (anti-Balb/c) central memory CD45.1⁺CD8⁺ T cells were generated in vitro and a portion of these cells were treated with pertussis toxin (PTX), which irreversibly inactivates Gαᵢ-coupled chemokine receptor signaling. PTX-treated or untreated central memory CD8⁺ T cells were injected into immunosuppressed B6 recipients of Balb/c lungs and analyzed by flow cytometry two days later. It was found that PTX treatment significantly impairs migration of donor-specific central memory CD8⁺ T lymphocytes into lung allograft tissue (FIG. 6A-C). By contrast, PTX treatment did not alter trafficking of third-party specific (anti-CBA/Ca) central memory CD8⁺ T cells into Balb/c allografts. Furthermore, compared to donor-specific cells, significantly fewer third party-specific central memory CD8⁺ T lymphocytes infiltrated Balb/c lung allograft tissue (FIG. 6A-C). This was not due to a global defect in cell migration as similar numbers of anti-Balb/c and anti-CBA/Ca CD45.1⁺ B6 central memory CD8⁺ T cells infiltrated spleens of lung graft recipients (FIG. 11). Similar to central memory CD8⁺ T lymphocytes, graft infiltration of in vitro generated anti-Balb/c CD8⁺ effector memory T cells was impaired after PTX treatment (FIG. 6A-C). However, the absolute number of anti-donor effector memory T cells accumulating in the lung was significantly lower than anti-donor central memory T cells (FIG. 6A-C). Collectively, these data suggest that chemokine receptor signaling as well as alloantigen recognition play a role in graft infiltration by CD8⁺ central memory T lymphocytes.

### Example 6. C—C Chemokine Receptor Type 7⁺ (CCR7⁺) Expression on CD8⁺ T Cells is Critical for Lung Allograft Acceptance

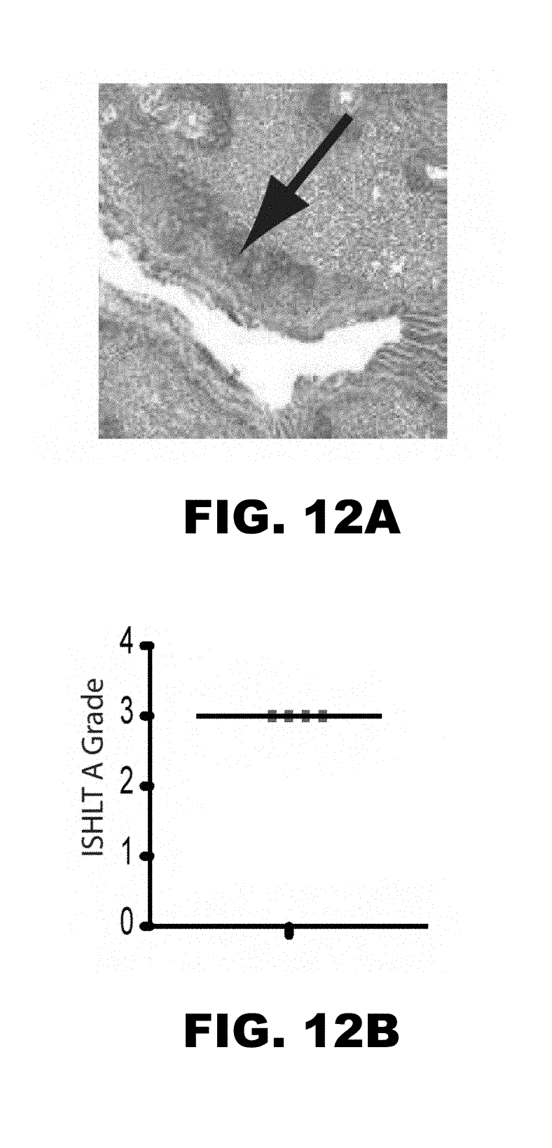

As the expression of the Gαᵢ-coupled chemokine receptor CCR7 is a hallmark of central memory T cells, and a large portion of CD8+CD44^{hi}CD62L^{hi} T cells in accepting lung allografts express CCR7 (FIG. 3R-V), it was next explored whether this specific chemokine receptor plays a role in graft acceptance. Balb/c lungs were first transplanted into immunosuppressed CCR7-deficient recipients and it was observed that these grafts were acutely rejected (FIG. 12A-B). As several cell populations in addition to T cells can express CCR7, T lymphocytes were focused on by adoptively transferring B6 CCR7-deficient CD8⁺ T lymphocytes into costimulatory blockade-treated B6 CD8^{-/-} recipients of Balb/c lungs. Unlike immunosuppressed CD8^{-/-} recipients reconstituted with wild-type CD8⁺ T lymphocytes (FIG. 2M-O), those reconstituted with CCR7^{-/-} CD8⁺ T cells acutely rejected Balb/c allografts (FIG. 6D-F). This demonstrates that CCR7 expression on recipient CD8 T cells plays a critical role in mediating lung allograft acceptance. Having demonstrated that IFN-γ production by recipient CD8⁺ T cells is essential for lung allograft acceptance, it was examined whether CCR7 expression on CD8 T cells regulates the production of this cytokine. Indeed, it was found that local expression of IFN-γ was significantly decreased when graft-infiltrating CD8 T cells lacked CCR7 (FIG. 6G).

It has been shown that graft-infiltrating recipient CD11c⁺ cells in rejecting lung allografts express both donor and self MHC Class II molecules on their surface and can activate CD4⁺ T cells via both direct and indirect allorecognition (55). Similarly, graft-infiltrating recipient CD11c⁺ dendritic cells in accepting lungs express both donor and recipient MHC Class I molecules (FIG. 6H, FIG. 6I), suggesting that recipient dendritic cells can contribute to the activation of alloreactive CD8⁺ T cells through both direct and indirect pathways of alloantigen presentation (56). Furthermore this cell population has been shown to express CCL21, a ligand for CCR7, on their surface (57). To further evaluate the role of CCR7 expression on CD8⁺ T cells, murine lungs were imaged by two-photon microscopy in vivo (58). Balb/c lungs were transplanted into immunosuppressed B6 CD11c− EYFP hosts that express enhanced yellow fluorescent protein under a CD11c promoter and injected fluorescently labeled B6 CD8⁺ wild-type and CCR7^{-/-} T cells three days after engraftment. When lung grafts were imaged 24 hours later, it was observed that wild-type CD8⁺ T lymphocytes made stable and long-lasting contacts with graft-infiltrating recipient CD11c⁺ cells. By contrast, in the absence of CCR7 expression, CD8⁺ T cells interacted with CD11c⁺ dendritic cells only briefly with significantly shorter retention times (FIG. 6J-P). Collectively, these findings suggest that in addition to directing trafficking of T lymphocytes into the lung, chemokine receptor signaling regulates contact between graft-infiltrating CD8⁺ T cells and alloantigen-expressing cells, which is associated with decreased local production of IFN-γ and graft rejection.

### Discussion for Examples 1-6

The overwhelming success of costimulation blockade in extending graft survival in small animal models of organ transplantation has laid the foundation for translating this therapy to the clinics (59). Kidney transplantation experiments in non-human primates, however, demonstrated that alloreactive memory T cells, generated through heterologous immunity, may represent a barrier to long-term graft survival in animals raised outside the confines of specific-pathogen free conditions (60, 61). This has been suspected to be especially problematic in recipients with a high frequency of CD8⁺ memory T cells due to rapid graft infiltration by this cell population (13, 15). Based on these observations strategies have been developed to either globally deplete T lymphocytes during the peri-operative period (62) or specifically target memory T cells (21). It has been reported that treatment of lung allograft recipients with CTLA4-Ig alone does not prevent acute rejection regardless of presence of CD4⁺ T cells (31). Additional treatment with anti-CD154 prevents rejection after transplantation of lungs into wild-type or even CD4⁺ T cell-depleted allogeneic hosts possibly due to transient expression of this costimulatory molecule on CD8⁺ T lymphocytes or other cells (13, 63).

The unique features of the lung, such as the rapidity and local initiation of the immune response, have allowed the discovery of a previously unrecognized and critical role for CD8⁺CD44^{hi}CD62L^{hi}CCR7⁺ T cells in the induction of graft acceptance. It has been shown that lungs provide a suitable environment for the activation of adaptive immunity in the absence of secondary lymphoid organs (83-85). Recent studies have demonstrated that innate and adaptive immune cells rapidly infiltrate lung grafts and that their interactions within the graft determine the fate of this organ (56, 78). Of particular relevance to the current findings, it has recently been shown that immune responses contributing to lung allograft acceptance are established locally in the graft shortly after transplantation (29) while other tissue and organ grafts require the presence of secondary lymphoid organs for the initiation as well as downregulation of alloimmune responses (64, 86, 87). The present findings with regard to trafficking requirements of CD8⁺ T cells to pulmonary allografts further extend the notion that lungs differ immunologically from other transplanted organs. It has been recently demonstrated that antigen recognition regulates trafficking of effector CD8⁺ T cells into murine heart grafts (63). Consistent with these data, this disclosure demonstrates that in vitro-generated central memory CD8⁺ T lymphocytes infiltrate lung allografts to a significantly larger extent compared to anti-third party central memory CD8⁺ T cells. In direct contrast to heart allografts, however, this disclosure demonstrates that Gα-1 receptor signaling is also critical for donor-primed CD8⁺ effector and central memory T cell infiltration into lung grafts. The disclosed findings extend recent reports that chemokine receptor expression on T cells regulates their homing to virally infected lungs (69). The present disclosure thus shows that both alloantigen and Gα1-dependent chemokine signaling play a role in memory T lymphocyte migration into lungs.

Since their description almost two decades ago (64), the majority of studies investigating mechanisms of immune regulation have focused on CD4⁺Foxp3⁺ regulatory T cells (65). Despite experimental evidence dating back to the 1970's that CD8⁺ T cells can suppress immune responses, only recently has this cell population experienced a resurgence in the literature. This is in large part due to the phenotypic heterogeneity of CD8⁺ T cells with suppressive function. To this end, CD8⁺ T cells with both naïve and memory phenotypes have been described to have regulatory capacity. Expansion of naïve human CD8⁺CCR7⁺ T cells with low-dose anti-CD3 and IL-15 induces their expression of Foxp3, CD25 and CD103 and their ability to suppress activation of CD4⁺ T cells (66). In mice, CD8⁺Foxp3⁺ T cells can regulate skin alloimmune responses in a contact-dependent fashion (43) and a similar population of cells that relies on direct interaction with CD4⁺ T cell responders has been described in man (66). In rats a regulatory CD8⁺Foxp3⁺CTLA4⁺CD45RC^{low} population has been described, however, controversy exists whether these cells suppress via production of cytokines or cell-to-cell contact (42, 67). There also exist reports that CD8⁺ T cells can suppress through TGF-β (48, 68). In contrast to these reports, the present disclosure describes an IFN-γ dependent mechanism of CD8⁺CCR7⁺ T cell-mediated immunosuppression in the murine lung.

CCR7 expression is a hallmark of central memory T cells and regulates their homing to lymph nodes. Investigations into the role of CCR7 in transplant rejection have yielded conflicting results, which may be in part due to this molecule regulating migration and function of multiple cell populations. Hearts and skin experienced a moderate prolongation in survival after transplantation into CCR7^{-/-} recipients, which is associated with reduced T cell graft infiltration (69). Interrupting CCR7 signaling has been shown to enhance allograft survival through reduction of Tₕ1 responses (70). Detrimental effects of recipient CCR7 deficiency on graft survival, on the other hand, have been attributed to decreased trafficking of tolerogenic antigen presenting cells, such as plasmacytoid dendritic cells, into draining lymph nodes (71) or CD4⁺Foxp3⁺ T cells into grafts (72). Here it is disclosed that CCR7-expressing CD8⁺ T cells are critical for lung allograft acceptance. Mechanistically, the present disclosure shows, by intravital two-photon microscopy, that in the absence of CCR7, CD8⁺ T cells are unable to form durable interactions with antigen presenting cells within the graft, which is associated with lower expression of IFN-γ. These findings extend previous reports showing that dendritic cells express CCL21 and that surface-bound CCR7 ligands induce tethering of T lymphocytes to antigen presenting cells during the formation of stable synapses (73, 74). It has also been shown that dendritic cells bind more CCR7 ligands on their surface than other cell populations (57). Previous reports have pointed to a role of CCR7 ligands in T cell differentiation. Stimulation of dendritic cells with CCR7 ligands induces their production of IL-12 and IL-23, which can drive Tₕ1 and Tₕ17 differentiation, respectively (75).

Traditionally, Tₕ1 responses have been considered to be instrumental in promoting cell-mediated rejection. An accumulation of IFN-γ producing CD4⁺ and CD8⁺ T cells in non-immunosuppressed lung allografts that undergo acute rejection has been described (31). Perhaps more importantly, excessive activation of Tₕ1 responses due to ischemia-reperfusion injury abrogates immunosuppression-mediated lung graft acceptance (77). However, the absence of IFN-γ can also have deleterious effects on graft survival. Cardiac allografts undergo necrosis in the absence of recipient IFN-γ despite immunosuppression, which has been attributed to inefficient deletion of activated T lymphocytes (78). Activation of alternative pathways, such as Tₕ17 differentiation, may also mediate aggressive pro-inflammatory responses in the absence of IFN-γ (79, 80).

As memory T lymphocytes in peripheral organs provide a first line of defense against infection, mucosal barrier organs such as the lung are especially rich in this cell population (81, 82). In fact, memory T cells are retained in lungs independent of antigen or inflammation, where they are rapid producers of pro-inflammatory cytokines (82, 83). As uncontrolled inflammatory responses in the lung can result in potentially life-threatening pulmonary dysfunction, mechanisms have evolved that limit the extent of inflammation to prevent tissue damage (84). For example, iNOS limits pulmonary inflammation in several models of lung injury (85, 86). It is thus possible that the costimulatory blockade protocol relies on a naturally occurring IFN-γ and NO dependent “feedback mechanism” normally operational in the lung. In contrast to central memory, effector memory CD8⁺ T cells do not promote lung allograft acceptance and are associated with graft rejection. Our findings support the notion that these two cell populations are functionally distinct (43). CCRT effector memory T cells rapidly infiltrate peripheral tissues during inflammation and are rich in effector molecules such as granzyme B and surface killer cell lectin-like receptor family members such as KLRG1 (94, 95). CCR7⁺ central memory T cells, however, have been traditionally described to reside in secondary lymphoid tissue and mediate delayed effector function through secretion of proinflammatory cytokines such as IFN-γ (48). It is thus possible that the unique physiology of the lung, which is enriched in central memory cells compared to other organs, relies on cytokine production by this cell population to downregulate immune responses. Since central memory CD8⁺ T cells that infiltrate accepting lung allografts have undergone proliferation we speculate that expansion of this cell population is needed to prevent rejection.

Our findings provide an impetus to critically evaluate current immunosuppressive strategies employed in clinical lung transplantation as many of them actually inhibit the pathways that we have identified as necessary for lung transplant acceptance. Examples include global T cell depletion, which would eliminate central memory CD8 T cells, mycophenolic acid, which inhibits early Tₕ1 responses (87) and calcineurin inhibitors that have been shown to suppress iNOS (88). New approaches such as ex vivo lung perfusion have the potential to test these findings in preclinical models (89).

Methods for the Examples

Animals.

Wild-type, IFN-γ^{-/-} IFN-γReceptor^{-/-}, CCR7^{-/-}, CD8^{-/-}, iNOS^{-/-}, TNFα^{-/-}, CD11c-EYFP, CD45.1⁺, B cell deficient (mu Ig^{-/-}) all on a B6 (H-2K^{b}) background, Balb/c (H-2K^{d}), CBA/Ca (H2K^{k}) and nude mice were purchased from The Jackson Laboratories (Bar Harbor, Me.). Animals were housed in a barrier facility in air-filtered cages. All studies were approved by the institutional animal studies committee. Left orthotopic vascularized lung transplants were performed as previously described (40) with costimulation blockade (CSB) in select experiments consisting of MR1 (250 μg intraperitoneally (i.p.) (day 0)) and CTLA4-Ig (200 μg i.p. (day 2)). As indicated for select experiments, CD8⁺ T cells were depleted in vivo by YTS 169.1 (250 μg i.p., days −3, −1), IFN-γ was neutralized using hamster-anti-mouse anti-IFN-γ antibody (Clone H22) (500 μg day −2, 250 μg day −1 i.p.), CD4⁺ T cells were depleted using GK1.5 (100 μg i.p. days −3, −1). For select experiments OX40-OX40 Ligand (clone OX-86), CD27-CD70 (clone FR-70) and ICOS-ICOS Ligand (clone 17G9) pathways were inhibited as previously described (all antibodies from BioXcell, Lebanon, N.H.) (41). For some experiments nude mice were reconstituted with 10⁷ CD4⁺ T cells isolated from the spleens and peripheral lymph nodes of B6 wild-type mice and for others CFSE-labeled CD4+CD45.1⁺ T cells were adoptively transferred into B6 mice. Reconstitution of B6 CD8^{-/-} mice was performed with a minimum of 5×10⁶ CD8⁺ T cells isolated either by flow cytometric sorting or magnetic bead isolation (Miltenyi Biotech, Auburn, Calif.).

Memory Cell Generation and Injection.

Both central and effector memory CD8⁺ T cells were generated in vitro based on previously described methods (50, 51). Briefly, central memory cells were generated by co-culturing B6 CD45.1⁺ splenocytes with irradiated Balb/c (donor) or CBA/Ca (third party) splenocyte stimulators. Sixty hours after initiation of the co-cultures dead cells were removed by Ficoll-Paque density centrifugation and CD8⁺ T cells were positively selected with magnetic beads. CD8⁺ cells were then expanded in 20 ng/ml IL-15 (R+D Systems, Minneapolis, Minn.) and injected intravenously approximately 2 weeks later. Effector memory cells were generated by co-culturing B6 CD45.1⁺ splenocytes with irradiated Balb/c stimulators in the presence of 1000 U/ml IL-2 (NIH NCI-Clinical Repository, Bethesda Mass.). For homing studies 5×10⁶ effector memory and 1×10⁶ central memory cells were injected per mouse two to three days after transplantation. For reconstitution experiments B6 CD8^{-/-} mice were injected with 5×10⁶ effector or central memory cells 48 to 72 hours prior to Balb/c lung allograft transplantation. For some experiments memory cells were treated with pertussis toxin at 200 ng/ml for 30 minutes prior to injection.

Histology.

Transplanted mouse lungs were fixed in formaldehyde, sectioned and stained with Hematoxylin and Eosin. A lung pathologist (JHR) blinded to the experimental conditions graded graft rejection using standard criteria (International Society for Heart and Lung Transplantation (ISHLT) A Grade) developed by the Lung Rejection Study Group (32).

Flow Cytometry.

All antibodies for flow cytometry were primarily fluorochrome-conjugated and purchased from eBioscience (San Diego, Calif.). Intracellular staining was performed as previously described (31).

In Vitro Mixed Lymphocyte Reactions.

In vitro mixed lymphocyte reactions were performed in round bottom 96-well plates using 3×10⁵ T cell-depleted Balb/c splenocyte stimulators with 10⁵ CFSE-labeled B6 CD45.1⁺ CD4⁺ or CD8⁺ T cell responders and, as indicated, 10⁵ CD8⁺ T cells isolated from lungs or spleens of immunosuppressed B6 recipients of Balb/c allografts. For some experiments central and effector memory T cells were sorted from lungs of resting mice. T cell responses were evaluated flow cytometrically on day 5. All compounds inhibiting the metabolism of essential amino acids were obtained from Sigma-Aldrich (St. Louis, Mo.) and added to the co-cultures as previously described for the duration of the experiment (41).

Quantitative Gene Expression Analysis.

For quantitative gene expression analysis mRNA from whole lung grafts was isolated in accordance with the manufacturer's instructions. Quantitative real-time RT-PCR was conducted on an ABI 7900 using TaqMan Gene Expression Assay system (Applied Biosystems) in accordance with the manufacturer's recommendations. Amplification of target sequences was conducted as follows: 50° C. for 20 minutes and 95° C. for 10 minutes, followed by 38-45 cycles of 95° C. for 15 seconds and 60° C. for 1 minute. Primers and MGB probes were purchased as kits from Applied Biosystems and can be identified in the following manner: IFN-γ (Mm01168134_ml), β-2 microglobulin (Mm00437762_ml).

NO Measurement In Vivo.

In vivo experiments were carried out using a 2 mm NO Sensor (World Precision Instruments) connected to a Free Radical Analyzer TBR-1025 (World Precision Instruments). The specifications include a 2 pA/nM sensitivity with a 1 nM minimum detection limit. Prior to the experiments, the sensor was polarized for at least 24 hours before use according to the manufacturer's recommendation. After sedating the mouse, a 2 mm long and 1 mm deep incision was made in the lung tissue to provide an area for the sensor to rest in. Approximately 0.5 mL of saline was applied to the incision in order to provide an interface between the mouse lung and sensor and also to monitor the integrity of the sensor's NO-selective membrane. The data from each lung were recorded using a LabTrax data acquisition unit and LabScribe software for 5 minutes after reaching a stable signal. The data were then analyzed against a baseline signal from normal saline and converted from current to NO concentration in ppm using NO donor DEA-Nonoate (Cayman Chemical) dissolved in PBS buffer as a standard.

Immunostaining.

Lungs were cryopreserved and then cut into 6-tm-thick sections. Sections were fixed in pure acetone for 10 min at −20° C. and blocked with 10% normal donkey serum. Unlabeled anti-CD4 (H129.19) and anti-CD8 (53-6.7) (Pharmingen (San Jose, Calif.)) were visualized using donkey anti-rat IgG conjugated with indocarbocyanine (Cy3) (Roche (Indianapolis, Ind.)). Slides were imaged using an Olympus BX51 microscope. No detectable staining was observed with isotype-matched or species-specific control antibodies.

Intravital 2-Photon Microscopy.

Balb/c lungs were transplanted into immunosuppressed B6 CD11c-EYFP recipients and on post-operative day 3 received an injection of 10⁷ CMTMR-labeled CCR7^{-/-} and 10⁷ CD8⁺ T cells isolated from wild-type B6 mice expressing cyan fluorescent protein (CFP) under an actin promoter. Time-lapse imaging was performed 24 hours after injection of T cells with a custom-built 2-photon microscope running ImageWarp Version 2.1 acquisition software (A&B Software). For time-lapse imaging of T cell-CD11c⁺ dendritic cell interactions in lung tissue, we averaged 15 video-rate frames (0.5 seconds per slice) during the acquisition to match the ventilator rate and to minimize movement artifacts. Each plane represents an image of 220×240 μm in the x and y dimensions. Twenty-one sequential planes were acquired in the z dimension (2.5 μm each) to form a z stack. Each individual T cell was tracked from its first appearance in the imaging window and followed up to the time point where it dislocated more than 20 μm from its starting position. T cells that did not travel were tracked for the duration of the imaging period.

Statistical Analysis.

Continuous variables such as in vitro and in vivo T cell proliferation, gene expression levels, retention times of T cells, number of memory T cells penetrating lung grafts as well as NO levels were compared between various conditions. Student t test was used for two comparisons and ANOVA for multiple comparisons as indicated in the appropriate figure. For ordinal variables, such as lung allograft rejection scores, the Mantel-Haenszel Chi-Square test was used. Data in figures are represented as mean±standard error of the mean. A p value of >0.05 is assumed to be not statistically significant.

Study Approval.

All animal procedures were approved by the Animal Studies Committee at Washington University School of Medicine, St. Louis, Mo.

### REFERENCES FOR THE EXAMPLES

1. Serraino, D., Piselli, P., Busnach, G., Burra, P., Citterio, F., Arbustini, E., Baccarani, U., De Juli, E., Pozzetto, U., Bellelli, S., et al. 2007. Risk of cancer following immunosuppression in organ transplant recipients and in HIV-positive individuals in southern Europe. Eur J Cancer 43:2117-2123.
2. Cervera, C., Linares, L., Bou, G., and Moreno, A. 2012. Multidrug-resistant bacterial infection in solid organ transplant recipients. Enferm Infecc Microbiol Clin 30 Suppl 2:40-48.
3. Larsen, C. P., Elwood, E. T., Alexander, D. Z., Ritchie, S. C., Hendrix, R., Tucker-Burden, C., Cho, H. R., Aruffo, A., Hollenbaugh, D., Linsley, P. S., et al. 1996. Long-term acceptance of skin and cardiac allografts after blocking CD40 and CD28 pathways. Nature 381:434-438.
4. Banuelos, S. J., Markees, T. G., Phillips, N. E., Appel, M. C., Cuthbert, A., Leif, J., Mordes, J. P., Shultz, L. D., Rossini, A. A., and Greiner, D. L. 2004. Regulation of skin and islet allograft survival in mice treated with costimulation blockade is mediated by different CD4+ cell subsets and different mechanisms. Transplantation 78:660-667.
5. Markees, T. G., Phillips, N. E., Gordon, E. J., Noelle, R. J., Shultz, L. D., Mordes, J. P., Greiner, D. L., and Rossini, A. A. 1998. Long-term survival of skin allografts induced by donor splenocytes and anti-CD154 antibody in thymectomized mice requires CD4(+) T cells, interferon-gamma, and CTLA4. J Clin Invest 101:2446-2455.
6. Mease, P., Genovese, M. C., Gladstein, G., Kivitz, A. J., Ritchlin, C., Tak, P. P., Wollenhaupt, J., Bahary, O., Becker, J. C., Kelly, S., et al. 2011. Abatacept in the treatment of patients with psoriatic arthritis: results of a six-month, multicenter, randomized, double-blind, placebo-controlled, phase II trial. Arthritis Rheum 63:939-948.
7. Schiff, M. 2011. Abatacept treatment for rheumatoid arthritis. Rheumatology (Oxford) 50:437-449.
8. Vincenti, F., Charpentier, B., Vanrenterghem, Y., Rostaing, L., Bresnahan, B., Darji, P., Massari, P., Mondragon-Ramirez, G. A., Agarwal, M., Di Russo, G., et al. 2011. A phase III study of belatacept-based immunosuppression regimens versus cyclosporine in renal transplant recipients (BENEFIT study). Am J Transplant 10:535-546.
9. Kreisel, D., Krupnick, A. S., Puri, V., Guthrie, T. J., Trulock, E. P., Meyers, B. F., and Patterson, G. A. 2011. Short- and long-term outcomes of 1000 adult lung transplant recipients at a single center. J Thorac Cardiovasc Surg 141:215-222.
10. Shah, P. D., and McDyer, J. F. 2010. Viral infections in lung transplant recipients. Semin Respir Crit Care Med 31:243-254.
11. Husain, S., Raza, K., Pilewski, J. M., Zaldonis, D., Crespo, M., Toyoda, Y., Shutt, K., Spichty, K., Bentlejewski, C., Pakstis, D. L., et al. 2009. Experience with immune monitoring in lung transplant recipients: correlation of low immune function with infection. Transplantation 87:1852-1857.
12. Adams, A. B., Williams, M. A., Jones, T. R., Shirasugi, N., Durham, M. M., Kaech, S. M., Wherry, E. J., Onami, T., Lanier, J. G., Kokko, K. E., et al. 2003. Heterologous immunity provides a potent barrier to transplantation tolerance. J Clin Invest 111:1887-1895.
13. Zhai, Y., Meng, L., Gao, F., Busuttil, R. W., and Kupiec-Weglinski, J. W. 2002. Allograft rejection by primed/memory CD8⁺ T cells is CD154 blockade resistant: therapeutic implications for sensitized transplant recipients. J Immunol 169:4667-4673.
14. Trambley, J., Bingaman, A. W., Lin, A., Elwood, E. T., Waitze, S. Y., Ha, J., Durham, M. M., Corbascio, M., Cowan, S. R., Pearson, T. C., et al. 1999. Asialo GM1(+) CD8(+) T cells play a critical role in costimulation blockade-resistant allograft rejection. J Glin Invest 104:1715-1722.
15. Schenk, A. D., Nozaki, T., Rabant, M., Valujskikh, A., and Fairchild, R. L. 2008. Donor-reactive CD8 memory T cells infiltrate cardiac allografts within 24-h posttransplant in naive recipients. Am J Transplant 8:1652-1661.
16. Schenk, A. D., Gorbacheva, V., Rabant, M., Fairchild, R. L., and Valujskikh, A. 2009. Effector functions of donor-reactive CD8 memory T cells are dependent on ICOS induced during division in cardiac grafts. Am J Transplant 9:64-73.
17. Koyama, I., Nadazdin, O., Boskovic, S., Ochiai, T., Smith, R. N., Sykes, M., Sogawa, H., Murakami, T., Strom, T. B., Colvin, R. B., et al. 2007. Depletion of CD8 memory T cells for induction of tolerance of a previously transplanted kidney allograft. Am J Transplant 7:1055-1061.
18. Donckier, V., Craciun, L., Miqueu, P., Troisi, R. I., Lucidi, V., Rogiers, X., Boon, N., Degre, D., Buggenhout, A., Moreno, C., et al. 2013. Expansion of memory-type CD8⁺ T cells correlates with the failure of early immunosuppression withdrawal after cadaver liver transplantation using high-dose ATG induction and rapamycin. Transplantation 96:306-315.
19. Ascon, M., Ascon, D. B., Liu, M., Cheadle, C., Sarkar, C., Racusen, L., Hassoun, H. T., and Rabb, H. 2009. Renal ischemia-reperfusion leads to long term infiltration of activated and effector-memory T lymphocytes. Kidney Int 75:526-535.
20. Lo, D. J., Weaver, T. A., Stempora, L., Mehta, A. K., Ford, M. L., Larsen, C. P., and Kirk, A. D. 2011. Selective targeting of human alloresponsive CD8+ effector memory T cells based on CD2 expression. Am J Transplant 11:22-33.
20. Weaver, T. A., Charafeddine, A. H., Agarwal, A., Turner, A. P., Russell, M., Leopardi, F. V., Kampen, R. L., Stempora, L., Song, M., Larsen, C. P., et al. 2009. Alefacept promotes co-stimulation blockade based allograft survival in nonhuman primates. Nat Med 15:746-749.
21. Dai, H., Wan, N., Zhang, S., Moore, Y., Wan, F., and Dai, Z. 2010. Cutting edge: programmed death-1 defines CD8+CD122+ T cells as regulatory versus memory T cells. J Immunol 185:803-807.
22. Bahri, R., Bollinger, A., Bollinger, T., Orinska, Z., and Bulfone-Paus, S. 2012. Ectonucleotidase CD38 demarcates regulatory, memory-like CD8+ T cells with IFN-gamma-mediated suppressor activities. PLoS One 7:e45234.
23. Dubois, A., Deruytter, N., Adams, B., Kanda, A., Delbauve, S., Fleury, S., Torres, D., Francois, A., Petein, M., Goldman, M., et al. 2010. Regulation of Th2 responses and allergic inflammation through bystander activation of CD8+ T lymphocytes in early life. J Immunol 185:884-891.
24. Berry, G. J., Angelini, A., Burke, M. M., Bruneval, P., Fishbein, M. C., Hammond, E., Miller, D., Neil, D., Revelo, M. P., Rodriguez, E. R., et al. 2011. The ISHLT working formulation for pathologic diagnosis of antibody-mediated rejection in heart transplantation: evolution and current status (2005-2011). J Heart Lung Transplant 30:601-611.
26. Maier, S., Tertilt, C., Chambron, N., Gerauer, K., Huser, N., Heidecke, C. D., and Pfeffer, K. 2001. Inhibition of natural killer cells results in acceptance of cardiac allografts in CD28−/− mice. Nat Med 7:557-562.
27. McNerney, M. E., Lee, K. M., Zhou, P., Molinero, L., Mashayekhi, M., Guzior, D., Sattar, H., Kuppireddi, S., Wang, C. R., Kumar, V., et al. 2006. Role of natural killer cell subsets in cardiac allograft rejection. Am J Transplant 6:505-513.
28. Nozaki, T., Amano, H., Bickerstaff, A., Orosz, C. G., Novick, A. C., Tanabe, K., and Fairchild, R. L. 2007. Antibody-mediated rejection of cardiac allografts in CCR5-deficient recipients. J Immunol 179:5238-5245.
29. Okazaki, M., Gelman, A. E., Tietjens, J. R., Ibricevic, A., Kornfeld, C. G., Huang, H. J., Richardson, S. B., Lai, J., Garbow, J. R., Patterson, G. A., et al. 2007. Maintenance of airway epithelium in acutely rejected orthotopic vascularized mouse lung transplants. Am J Respir Cell Mol Biol 37:625-630.
30. Li, W., Bribriesco, A. C., Nava, R. G., Brescia, A. A., Ibricevic, A., Spahn, J. H., Brody, S. L., Ritter, J. H., Gelman, A. E., Krupnick, A. S., et al. 2012. Lung transplant acceptance is facilitated by early events in the graft and is associated with lymphoid neogenesis. Mucosal Immunol 5:544-554.
31. Gelman, A. E., Okazaki, M., Lai, J., Kornfeld, C. G., Kreisel, F. H., Richardson, S. B., Sugimoto, S., Tietjens, J. R., Patterson, G. A., Krupnick, A. S., et al. 2008. CD4⁺ T lymphocytes are not necessary for the acute rejection of vascularized mouse lung transplants. J Immunol 180:4754-4762.
32. Yousem, S. A., Berry, G. J., Cagle, P. T., Chamberlain, D., Husain, A. N., Hruban, R. H., Marchevsky, A., Ohori, N. P., Ritter, J., Stewart, S., et al. 1996. Revision of the 1990 working formulation for the classification of pulmonary allograft rejection: Lung Rejection Study Group. J Heart Lung Transplant 15:1-15.
33. Yamamoto, S., Nava, R. G., Zhu, J., Huang, H. J., Ibrahim, M., Mohanakumar, T., Miller, M. J., Krupnick, A. S., Kreisel, D., and Gelman, A. E. 2012. Cutting edge: Pseudomonas aeruginosa abolishes established lung transplant tolerance by stimulating B7 expression on neutrophils. J Immunol 189:4221-4225.
34. Rigby, M. R., Trexler, A. M., Pearson, T. C., and Larsen, C. P. 2008. CD28/CD154 blockade prevents autoimmune diabetes by inducing nondeletional tolerance after effector t-cell inhibition and regulatory T-cell expansion. Diabetes 57:2672-2683.
35. Waldmann, H., and Cobbold, S. 2001. Regulating the immune response to transplants. a role for CD4+ regulatory cells? Immunity 14:399-406.
36. Lee, I., Wang, L., Wells, A. D., Dorf, M. E., Ozkaynak, E., and Hancock, W. W. 2005. Recruitment of Foxp3+ T regulatory cells mediating allograft tolerance depends on the CCR4 chemokine receptor. J Exp Med 201:1037-1044.
37. Iwakoshi, N. N., Mordes, J. P., Markees, T. G., Phillips, N. E., Rossini, A. A., and Greiner, D. L. 2000. Treatment of allograft recipients with donor-specific transfusion and anti-CD154 antibody leads to deletion of alloreactive CD8+ T cells and prolonged graft survival in a CTLA4-dependent manner. J Immunol 164:512-521.
38. Walsh, P. T., Taylor, D. K., and Turka, L. A. 2004. Tregs and transplantation tolerance. J Clin Invest 114:1398-1403.
39. Redfield, R. R., 3rd, Rodriguez, E., Parsons, R., Vivek, K., Mustafa, M. M., Noorchashm, H., and Naji, A. 2011. Essential role for B cells in transplantation tolerance. Curr Opin Immunol 23:685-691.
40. Okazaki, M., Krupnick, A. S., Kornfeld, C. G., Lai, J. M., Ritter, J. H., Richardson, S. B., Huang, H. J., Das, N. A., Patterson, G. A., Gelman, A. E., et al. 2007. A mouse model of orthotopic vascularized aerated lung transplantation. Am J Transplant 7:1672-1679.
41. Habicht, A., Najafian, N., Yagita, H., Sayegh, M. H., and Clarkson, M. R. 2007. New insights in CD28-independent allograft rejection. Am J Transplant 7:1917-1926.
42. Xystrakis, E., Dejean, A. S., Bernard, I., Druet, P., Liblau, R., Gonzalez-Dunia, D., and Saoudi, A. 2004. Identification of a novel natural regulatory CD8 T-cell subset and analysis of its mechanism of regulation. Blood 104:3294-3301.
43. Lerret, N. M., Houlihan, J. L., Kheradmand, T., Pothoven, K. L., Zhang, Z. J., and Luo, X. 2012. Donor-specific CD8+ Foxp3+ T cells protect skin allografts and facilitate induction of conventional CD4+ Foxp3+ regulatory T cells. Am J Transplant 12:2335-2347.
44. Correale, J., and Villa, A. 2010. Role of CD8+ CD25+ Foxp3+ regulatory T cells in multiple sclerosis. Ann Neurol 67:625-638.
45. Sallusto, F., Lenig, D., Forster, R., Lipp, M., and Lanzavecchia, A. 1999. Two subsets of memory T lymphocytes with distinct homing potentials and effector functions. Nature 401:708-712.
46. Ford, M. L., Kirk, A. D., and Larsen, C. P. 2009. Donor-reactive T-cell stimulation history and precursor frequency: barriers to tolerance induction. Transplantation 87:S69-74.
47. Bingaman, A. W., and Farber, D. L. 2004. Memory T cells in transplantation: generation, function, and potential role in rejection. Am J Transplant 4:846-852.
48. Wan, N., Dai, H., Wang, T., Moore, Y., Zheng, X. X., and Dai, Z. 2008. Bystander central memory but not effector memory CD8+ T cells suppress allograft rejection. J Immunol 180:113-121.
49. Ely, K. H., Cookenham, T., Roberts, A. D., and Woodland, D. L. 2006. Memory T cell populations in the lung airways are maintained by continual recruitment. J Immunol 176:537-543.
50. Weninger, W., Crowley, M. A., Manjunath, N., and von Andrian, U. H. 2001. Migratory properties of naive, effector, and memory CD8(+) T cells. J Exp Med 194:953-966.
51. Ophir, E., Or-Geva, N., Gurevich, I., Tal, O., Eidelstein, Y., Shezen, E., Margalit, R., Lask, A., Shakhar, G., Hagin, D., et al. 2012. Murine anti-third-party central-memory CD8(+) T cells promote hematopoietic chimerism under mild conditioning: lymph-node sequestration and deletion of anti-donor T cells. Blood 121:1220-1228.
52. Cobbold, S. P., Adams, E., Farquhar, C. A., Nolan, K. F., Howie, D., Lui, K. O., Fairchild, P. J., Mellor, A. L., Ron, D., and Waldmann, H. 2009. Infectious tolerance via the consumption of essential amino acids and mTOR signaling. Proc Natl Acad Sci USA 106:12055-12060.
53. Geller, D. A., Nussler, A. K., Di Silvio, M., Lowenstein, C. J., Shapiro, R. A., Wang, S. C., Simmons, R. L., and Billiar, T. R. 1993. Cytokines, endotoxin, and glucocorticoids regulate the expression of inducible nitric oxide synthase in hepatocytes. Proc Natl Acad Sci USA 90:522-526.
54. Purushothaman, D., Marcel, N., Garg, M., Venkataraman, R., and Sarin, A. 2013. Apoptotic programs are determined during lineage commitment of CD4+ T effectors: selective regulation of T effector-memory apoptosis by inducible nitric oxide synthase. J Immunol 190:97-105.
55. Gelman, A. E., Okazaki, M., Sugimoto, S., Li, W., Kornfeld, C. G., Lai, J., Richardson, S. B., Kreisel, F. H., Huang, H. J., Tietjens, J. R., et al. 2010. CCR2 regulates monocyte recruitment as well as CD4 T1 allorecognition after lung transplantation. Am J Transplant 10:1189-1199.
56. Rogers, N. J., and Lechler, R. I. 2001. Allorecognition. Am J Transplant 1:97-102.
57. Friedman, R. S., Jacobelli, J., and Krummel, M. F. 2006. Surface-bound chemokines capture and prime T cells for synapse formation. Nat Immunol 7:1101-1108.
58. Kreisel, D., Nava, R. G., Li, W., Zinselmeyer, B. H., Wang, B., Lai, J., Pless, R., Gelman, A. E., Krupnick, A. S., and Miller, M. J. 2010. In vivo two-photon imaging reveals monocyte-dependent neutrophil extravasation during pulmonary inflammation. Proc Natl Acad Sci USA 107:18073-18078.
59. Vincenti, F., Larsen, C. P., Alberu, J., Bresnahan, B., Garcia, V. D., Kothari, J., Lang, P., Urrea, E. M., Massari, P., Mondragon-Ramirez, G., et al. 2012. Three-year outcomes from BENEFIT, a randomized, active-controlled, parallel-group study in adult kidney transplant recipients. Am J Transplant 12:210-217.
60. Lo, D. J., Anderson, D. J., Weaver, T. A., Leopardi, F., Song, M., Farris, A. B., Strobert, E. A., Jenkins, J., Turgeon, N. A., Mehta, A. K., et al. 2013. Belatacept and sirolimus prolong nonhuman primate renal allograft survival without a requirement for memory T cell depletion. Am J Transplant 13:320-328.
61. Nadazdin, O., Boskovic, S., Murakami, T., Tocco, G., Smith, R. N., Colvin, R. B., Sachs, D. H., Allan, J., Madsen, J. C., Kawai, T., et al. 2011. Host alloreactive memory T cells influence tolerance to kidney allografts in nonhuman primates. Sci Transl Med 3:86ra51.
62. Padiyar, A., Augustine, J. J., and Hricik, D. E. 2009. Induction antibody therapy in kidney transplantation. Am J Kidney Dis 54:935-944.
63. Mackey, M. F., Barth, R. J., Jr., and Noelle, R. J. 1998. The role of CD40/CD154 interactions in the priming, differentiation, and effector function of helper and cytotoxic T cells. J Leukoc Biol 63:418-428.
64. Sakaguchi, S., Sakaguchi, N., Asano, M., Itoh, M., and Toda, M. 1995. Immunologic self-tolerance maintained by activated T cells expressing IL-2 receptor alpha-chains (CD25). Breakdown of a single mechanism of self-tolerance causes various autoimmune diseases. J Immunol 155:1151-1164.
65. Zhang, N., Schroppel, B., Lal, G., Jakubzick, C., Mao, X., Chen, D., Yin, N., Jessberger, R., Ochando, J. C., Ding, Y., et al. 2009. Regulatory T cells sequentially migrate from inflamed tissues to draining lymph nodes to suppress the alloimmune response. Immunity 30:458-469.
66. Suzuki, M., Jagger, A. L., Konya, C., Shimojima, Y., Pryshchep, S., Goronzy, J. J., and Weyand, C. M. 2012. CD8+CD45RA+CCR7+FOXP3+ T cells with immunosuppressive properties: a novel subset of inducible human regulatory T cells. J Immunol 189:2118-2130.
67. Guillonneau, C., Hill, M., Hubert, F. X., Chiffoleau, E., Herve, C., Li, X. L., Heslan, M., Usal, C., Tesson, L., Menoret, S., et al. 2007. CD40Ig treatment results in allograft acceptance mediated by CD8CD45RC T cells, IFN-gamma, and indoleamine 2,3-dioxygenase. J Clin Invest 117:1096-1106.
68. Myers, L., Croft, M., Kwon, B. S., Mittler, R. S., and Vella, A. T. 2005. Peptide-specific CD8 T regulatory cells use IFN-gamma to elaborate TGF-beta-based suppression. J Immunol 174:7625-7632.
69. Beckmann, J. H., Yan, S., Luhrs, H., Heid, B., Skubich, S., Forster, R., and Hoffmann M. W. 2004. Prolongation of allograft survival in ccr7-deficient mice. Transplantation 77:1809-1814.
70. Ziegler, E., Gueler, F., Rong, S., Mengel, M., Witzke, O., Kribben, A., Haller, H., Kunzendorf, U., and Krautwald, S. 2006. CCL19-IgG prevents allograft rejection by impairment of immune cell trafficking. J Am Soc Nephrol 17:2521-2532.
71. Liu, X., Mishra, P., Yu, S., Beckmann, J., Wendland, M., Kocks, J., Seth, S., Hoffmann, K., Hoffmann, M., Kremmer, E., et al. 2011. Tolerance induction towards cardiac allografts under costimulation blockade is impaired in CCR7-deficient animals but can be restored by adoptive transfer of syngeneic plasmacytoid dendritic cells. Eur J Immunol 41:611-623.
72. Gregson, A. L., Hoji, A., Palchevskiy, V., Hu, S., Weigt, S. S., Liao, E., Derhovanessian, A., Saggar, R., Song, S., Elashoff, R., et al. 2010. Protection against bronchiolitis obliterans syndrome is associated with allograft CCR7+ CD45RA− T regulatory cells. PLoS One 5:e11354.
73. Kaiser, A., Donnadieu, E., Abastado, J. P., Trautmann, A., and Nardin, A. 2005. CC chemokine ligand 19 secreted by mature dendritic cells increases naive T cell scanning behavior and their response to rare cognate antigen. J Immunol 175:2349-2356.
74. Ngo, V. N., Tang, H. L., and Cyster, J. G. 1998. Epstein-Barr virus-induced molecule 1 ligand chemokine is expressed by dendritic cells in lymphoid tissues and strongly attracts naive T cells and activated B cells. J Exp Med 188:181-191.
75. Kuwabara, T., Ishikawa, F., Yasuda, T., Aritomi, K., Nakano, H., Tanaka, Y., Okada, Y., Lipp, M., and Kakiuchi, T. 2009. CCR7 ligands are required for development of experimental autoimmune encephalomyelitis through generating IL-23-dependent Th17 cells. J Immunol 183:2513-2521.
76. Noor, S., Habashy, A. S., Nance, J. P., Clark, R. T., Nemati, K., Carson, M. J., and Wilson, E. H. 2010. CCR7-dependent immunity during acute Toxoplasma gondii infection. Infect Immun 78:2257-2263.
77. Kreisel, D., Sugimoto, S., Zhu, J., Nava, R., Li, W., Okazaki, M., Yamamoto, S., Ibrahim, M., Huang, H. J., Toth, K. A., et al. 2011. Emergency granulopoiesis promotes neutrophil-dendritic cell encounters that prevent mouse lung allograft acceptance. Blood 118:61726182.
78. Konieczny, B. T., Dai, Z., Elwood, E. T., Saleem, S., Linsley, P. S., Baddoura, F. K., Larsen, C. P., Pearson, T. C., and Lakkis, F. G. 1998. IFN-gamma is critical for long-term allograft survival induced by blocking the CD28 and CD40 ligand T cell costimulation pathways. J Immunol 160:2059-2064.
79. Yuan, X., Paez-Cortez, J., Schmitt-Knosalla, I., D'Addio, F., Mfarrej, B., Donnarumma, M., Habicht, A., Clarkson, M. R., Iacomini, J., Glimcher, L. H., et al. 2008. A novel role of CD4 Th17 cells in mediating cardiac allograft rejection and vasculopathy. J Exp Med 205:3133-3144.
80. Sis, B., Famulski, K. S., Allanach, K. L., Zhu, L. F., and Halloran, P. F. 2007. IFN-gamma prevents early perforin-granzyme-mediated destruction of kidney allografts by inducing donor class I products in the kidney. Am J Transplant 7:2301-2310.
81. Sheridan, B. S., and Lefrancois, L. 2011. Regional and mucosal memory T cells. Nat Immunol 12:485-491.
82. Sathaliyawala, T., Kubota, M., Yudanin, N., Turner, D., Camp, P., Thome, J. J., Bickham, K. L., Lerner, H., Goldstein, M., Sykes, M., et al. 2013. Distribution and compartmentalization of human circulating and tissue-resident memory T cell subsets. Immunity 38:187-197.
83. Teijaro, J. R., Turner, D., Pham, Q., Wherry, E. J., Lefrancois, L., and Farber, D. L. 2011. Cutting edge: Tissue-retentive lung memory CD4 T cells mediate optimal protection to respiratory virus infection. J Immunol 187:5510-5514.
84. Lentsch, A. B., and Ward, P. A. 2001. Regulation of experimental lung inflammation. Respir Physiol 128:17-22.
85. Zeidler, P. C., Millecchia, L. M., and Castranova, V. 2004. Role of inducible nitric oxide synthase-derived nitric oxide in lipopolysaccharide plus interferon-gamma-induced pulmonary inflammation. Toxicol Appl Pharmacol 195:45-54.
86. Kobayashi, H., Hataishi, R., Mitsufuji, H., Tanaka, M., Jacobson, M., Tomita, T., Zapol, W. M., and Jones, R. C. 2001. Antiinflammatory properties of inducible nitric oxide synthase in acute hyperoxic lung injury. Am J Respir Cell Mol Biol 24:390-397.
87. He, X., Smeets, R. L., Koenen, H. J., Vink, P. M., Wagenaars, J., Boots, A. M., and Joosten, I. 2011. Mycophenolic acid-mediated suppression of human CD4+ T cells: more than mere guanine nucleotide deprivation. Am J Transplant 11:439-449.
88. Dusting, G. J., Akita, K., Hickey, H., Smith, M., and Gurevich, V. 1999. Cyclosporin A and tacrolimus (FK506) suppress expression of inducible nitric oxide synthase in vitro by different mechanisms. Br J Pharmacol 128:337-344.89.
89. Cypel, M., Liu, M., Rubacha, M., Yeung, J. C., Hirayama, S., Anraku, M., Sato, M., Medin, J., Davidson, B. L., de Perrot, M., et al. 2009. Functional repair of human donor lungs by IL-10 gene therapy. Sci Transl Med 1:4ra9.

What is claimed is:

1. A method of inducing tolerance to a lung allograft in a subject, the method comprising suppressing an alloimmune response to the lung allograft in the subject by administering CD8⁺ T cells to the lung allograft in an amount sufficient to suppress the alloimmune response in the subject.

2. The method of claim 1, wherein the CD8⁺ T cells are CD8⁺ central memory T cells.

3. The method of claim 1, wherein the CD8⁺ T cells are CD8⁺ CCR7⁺ central memory T cells.

4. The method of claim 1, wherein the CD8+ central memory T cells are CD8+CD44hi CD62L^{hi}CCR7⁺ central memory T cells.

5. The method of claim 1, wherein the CD8⁺ T cells are recipient CD8⁺ T cells.

6. The method of claim 1, wherein the number of CD8⁺ T cells in the allograft is significantly increased when compared to the number of CD8⁺ T cells in a normal lung.

7. The method of claim 1, wherein the CD8⁺ T cells are administered by perfusing the lung allograft with the CD8⁺ T cells.

8. The method of claim 1, wherein the CD8⁺ T cells are administered to the lung allograft at least 72 hours prior to transplantation.

9. A method of inducing tolerance to a lung allograft in a subject by increasing the level of nitric oxide in the lung allograph of the subject, wherein the level of nitric oxide is increased by administering CD8⁺ T cells to the lung allograft.

10. The method of claim 9, wherein the level of nitric oxide in the allograft is significantly increased when compared to level of nitric oxide in a normal lung.

11. The method of claim 9, wherein the central memory T cells are CD8⁺ CCR7⁺ cells.

12. The method in claim 9, wherein the central memory T cells are CD8+CD44hi CD62Lhi CCR7+ cells.

13. The method of claim 9, wherein the CD8⁺ T cells are recipient CD8⁺ T cells.

14. The method of claim 9, wherein the CD8⁺ T cells are administered to the lung allograft at least 72 hours prior to transplantation.

15. A method of administering central memory T cells to an ex vivo graft, the method comprising,
a. obtaining immune cells from a subject;
b. culturing the immune cells in vitro;
c. generating central memory T cells by stimulating the immune cells in vitro; and
d. administering the central memory cells to the graft.

16. The method of claim 15, wherein the immune cells from the subject are allogeneic splenocytes.

17. The method of claim 15, wherein the immune cells are CD8+ T cells obtained from recipient CD8+ T cells.

18. The method of claim 15, wherein the ex vivo graft is a lung graft.
